SEQUENCE LISTING (28 sequences)

SEQ ID NO: 1 - DNA, 1425 bp, synthetic construct
  source 1..1425: mol_type = other DNA
atggccaaacagcaagaagcagaactgatttttatcccgtttccgattccgggtcatattctggcaaccattgaactggcaaaacgtctgattagccatcagccgagccgtattcataccattaccattctgcattggagcctgccgtttctgccgcagagcgataccattgcatttctgaaaagcctgattgaaaccgaaagtcgtattcgcctgattaccctgccggatgttcagaatccgcctccgatggaactgtttgttaaagcaagcgaaagctacatcctggaatacgtgaaaaaaatggttccgctggttcgtaatgcactgagcaccctgctgagcagccgtgatgaaagcgatagcgttcatgttgcaggtctggttctggattttttctgtgttccgctgattgatgtgggcaatgaatttaatctgccgagctatatctttctgacctgtagcgcaagctttctgggtatgatgaaatatctgctggaacgtaatcgtgaaaccaaaccggaactgaatcgtagcagtgatgaagaaaccattagcgttccgggttttgttaatagcgtgccggttaaagttctgcctccgggtctgtttaccaccgaaagctatgaagcatgggttgaaatggcagaacgttttccggaagcaaaaggtattctggtgaatagctttgaaagcctggaacgcaatgccttcgattattttgatcgtcgtccggataactatccgcctgtttatccgattggtccgattctgtgtagcaatgatcgtccgaatctggatctgagcgaacgtgatcgtattctgaaatggctggatgatcagccggaaagcagcgttgtgtttctgtgctttggtagcctgaaaagtctggcagcaagccagattaaagaaattgcacaggcactggaactggtgggtattcgttttctgtggtcaattcgtaccgatccgaaagaatatgcaagcccgaatgaaattctgcctgatggttttatgaatcgcgttatgggtctgggtttagtttgtggttgggcaccgcaggttgaaatcctggcacataaagcaattggtggttttgtgagccattgcggttggaatagcattctggaaagtctgcgttttggtgttccgattgcaacctggccgatgtatgcagaacagcagctgaatgcatttaccatcgttaaagaattaggtctggccctggaaatgcgtctggattatgttagcgaatatggcgaaattgtgaaagccgatgaaattgccggtgcagttcgtagcctgatggatggtgaagatgttccgcgtcgtaaactgaaagaaatcgcagaagcaggtaaagaagcagttatggatggcggtagcagctttgttgcagttaaacgttttattgatggcctgtaa

SEQ ID NO: 2 - protein, 474 aa, synthetic construct
MAKQQEAELIFIPFPIPGHILATIELAKRLISHQPSRIHTITILHWSLPFLPQSDTIAFLKSLIETESRIRLITLPDVQNPPPMELFVKASESYILEYVKKMVPLVRNALSTLLSSRDESDSVHVAGLVLDFFCVPLIDVGNEFNLPSYIFLTCSASFLGMMKYLLERNRETKPELNRSSDEETISVPGFVNSVPVKVLPPGLFTTESYEAWVEMAERFPEAKGILVNSFESLERNAFDYFDRRPDNYPPVYPIGPILCSNDRPNLDLSERDRILKWLDDQPESSVVFLCFGSLKSLAASQIKEIAQALELVGIRFLWSIRTDPKEYASPNEILPDGFMNRVMGLGLVCGWAPQVEILAHKAIGGFVSHCGWNSILESLRFGVPIATWPMYAEQQLNAFTIVKELGLALEMRLDYVSEYGEIVKADEIAGAVRSLMDGEDVPRRKLKEIAEAGKEAVMDGGSSFVAVKRFIDGL

SEQ ID NO: 3 - DNA, 1422 bp, synthetic construct
  source 1..1422: mol_type = other DNA
atggaagatgccattgttctgtatccgagtcctggtcgtggtcatctgattagcatggttgaactgggtaaactgatcctgaaaaatcatccgagctttagcattaccattctgattctgagcgaaccgaataccaatgcaaatagcaccgcaccgaattatgttgcaagcagcaccaccgaatatatcaccaccgttaatagcacaaccccgagcattacctttcatcatctgcctccgattagcgaagttccgcctagcaccagcagctttgttgaactgagctatctgattccgcgttttaacaatcagaacctgcatcagattctgaaaaccattagccagaccagcaaactgaaagcctttatcatcgattttttctgcgacgcagcatttgaagttgcaaccaatctggatattccgacctattatttctttaccagcggtgcatgttatctggcagcactgctgtatcgtccgacactgcataaaaaagttgacaaaagcctgaaagatctgggtaataccctgctggacattccgggtctgccaccgattcaggcaagcgatctgccggaaccgtggaaagatcgtaccagcaaagaatatcagtatagcctgaatgttgcagcccatatggcaaaaagcaatggtctgctggttaatacctttgatctgctggaaaccaaagccattaaagcaattagcggtggtctgtgtgttccggatcgtacaacccctccgattttttgtattggtccgctgattagcgatagcaatcaggatgttgaagatcatgaatgtctgaattggctgaatagccagccgagccgtagcgttgtttttctgtgttttggtagcctgggtctgtttagtgcaaaacagctgaaagaaattgcagttggtctggaaaatagcggtcagcgttttctgtgggttctgcgtaatcctccgagcgataatgataaagatccgaatctggatgaactgctgccgaaaggttttctggaacgtaccaaagaaaaaggctttgttgttaaacagtgggcaccgcaggttgcagttctgcgtcatgatagcgttggtggttttgttacccattgtggttggaatagcgttctggaagcagtttggtgtcgtgttccgatggttggttggcctctgtatgcagaacagcgtctgaatagcgtggttctggttgaagaaattaaactggcaagcggtctgaatgaaagcgaagatggtctggttagcgcagcagaactggaaaaacgtgttaaagaactgatggatagtgaagttggcaccgaagttcgtgaacgtgttagcggtctgcgtgatgaagcagttaccgcagttaaagaaggtggtagcagccacgttgcactggcaaaactggcagaactgtggcgtcagaattaa

SEQ ID NO: 4 - protein, 473 aa, synthetic construct
MEDAIVLYPSPGRGHLISMVELGKLILKNHPSFSITILILSEPNTNANSTAPNYVASSTTEYITTVNSTTPSITFHHLPPISEVPPSTSSFVELSYLIPRFNNQNLHQILKTISQTSKLKAFIIDFFCDAAFEVATNLDIPTYYFFTSGACYLAALLYRPTLHKKVDKSLKDLGNTLLDIPGLPPIQASDLPEPWKDRTSKEYQYSLNVAAHMAKSNGLLVNTFDLLETKAIKAISGGLCVPDRTTPPIFCIGPLISDSNQDVEDHECLNWLNSQPSRSVVFLCFGSLGLFSAKQLKEIAVGLENSGQRFLWVLRNPPSDNDKDPNLDELLPKGFLERTKEKGFVVKQWAPQVAVLRHDSVGGFVTHCGWNSVLEAVWCRVPMVGWPLYAEQRLNSVVLVEEIKLASGLNESEDGLVSAAELEKRVKELMDSEVGTEVRERVSGLRDEAVTAVKEGGSSHVALAKLAELWRQN

SEQ ID NO: 5 - DNA, 1404 bp, synthetic construct
  source 1..1404: mol_type = other DNA
atgcgtaatgtggaactgatttttatcccgacaccgaccgttggtcatctggttccgtttctggaatttgcacgtcgtctgattgaacaggatgatcgtattcgtattaccatcctgctgatgaaactgcagggtcagagccatctggatacctatgttaaaagcattgcaagcagccagccgtttgttcgttttattgatgtgccggaactggaagaaaaaccgacactgggtagcacccagagcgttgaagcatatgtttatgatgtgattgaacgcaatattccgctggtgcgtaatattgttatggatattctgaccagcctggcactggatggtgttaaagttaaaggtctggttgtggattttttctgcctgccgatgattgatgttgccaaagatattagcctgccgttttatgtttttctgaccaccaatagcggttttctggcaatgatgcagtatctggcagatcgtcatagccgtgataccagcgtttttgttcgtaatagcgaagaaatgctgagcattccgggttttgttaatccggttccggcaaatgttctgccgagcgcactgtttgttgaagatggttatgatgcgtatgttaaactggccatcctgtttaccaaagccaatggtattctggtgaatagcagctttgatatcgaaccgtatagcgtgaatcactttctgcaagaacagaattatccgagcgtttatgcagttggtccgatctttgatctgaaagcacagccgcatccggaacaggatctgacccgtcgtgatgaactgatgaaatggctggatgatcagccggaagcaagcgttgtgtttctgtgttttggtagcatggcacgtctgcgtggtagcctggttaaagaaattgcacatggtctggaactgtgccagtatcgttttctgtggtcactgcgtaaagaagaagttaccaaagacgacctgccggaaggctttctggatcgtgttgatggtcgtggtatgatttgtggttggagtccgcaggttgaaattctggcacataaagcagttggtggttttgtgagccattgcggttggaatagcattgttgaaagcctgtggtttggtgttccgattgttacctggccgatgtatgcagaacagcagctgaatgcatttctgatggtgaaagaactgaaactggcagttgaactgaagctggattatcgtgttcattccgatgaaattgtgaacgccaatgaaattgaaaccgccattcgttatgtgatggataccgataacaatgttgtgcgtaaacgtgtcatggatatcagccagatgattcagcgtgcaaccaaaaatggtggtagcagttttgcagccatcgagaaatttatctatgacgtgattggcatcaagccgtaa

SEQ ID NO: 6 - protein, 467 aa, synthetic construct
MRNVELIFIPTPTVGHLVPFLEFARRLIEQDDRIRITILLMKLQGQSHLDTYVKSIASSQPFVRFIDVPELEEKPTLGSTQSVEAYVYDVIERNIPLVRNIVMDILTSLALDGVKVKGLVVDFFCLPMIDVAKDISLPFYVFLTTNSGFLAMMQYLADRHSRDTSVFVRNSEEMLSIPGFVNPVPANVLPSALFVEDGYDAYVKLAILFTKANGILVNSSFDIEPYSVNHFLQEQNYPSVYAVGPIFDLKAQPHPEQDLTRRDELMKWLDDQPEASVVFLCFGSMARLRGSLVKEIAHGLELCQYRFLWSLRKEEVTKDDLPEGFLDRVDGRGMICGWSPQVEILAHKAVGGFVSHCGWNSIVESLWFGVPIVTWPMYAEQQLNAFLMVKELKLAVELKLDYRVHSDEIVNANEIETAIRYVMDTDNNVVRKRVMDISQMIQRATKNGGSSFAAIEKFIYDVIGIKP

SEQ ID NO: 7 - DNA, 1425 bp, synthetic construct
  source 1..1425: mol_type = other DNA
atgagcaccagcgaactggtttttattccgagtcctggtgcaggtcatctgcctccgaccgttgaactggcaaaactgctgctgcatcgtgatcagcgtctgagcgttaccattattgttatgaatctgtggctgggtccgaaacataataccgaagcacgtccgtgtgttccgagcctgcgttttgttgatattccgtgtgatgaaagcaccatggcactgattagcccgaatacctttattagcgcatttgtggaacatcataaaccgcgtgttcgtgatattgtgcgtggtattattgaaagcgatagcgttcgtctggcaggttttgttctggatatgttttgtatgccgatgagtgatgtggccaatgaatttggtgtgccgagctataactattttaccagcggtgcagcaaccctgggtctgatgtttcatctgcagtggaaacgtgatcatgaaggttatgatgcaaccgaactgaaaaatagcgataccgaactgtcagttccgagctatgttaatccggttccggcaaaagttctgcctgaagttgtgctggataaagaaggtggtagcaaaatgtttctggatctggcagaacgtattcgtgaaagcaaaggcattattgtgaatagctgtcaggcaattgaacgtcatgcactggaatatctgagcagcaataacaatggtattccgcctgtttttccggttggtccgattctgaatctggaaaacaaaaaagatgatgccaaaaccgatgaaattatgcgctggctgaatgaacagccggaaagcagcgttgtttttctgtgttttggtagcatgggcagctttaatgagaaacaggttaaagaaattgccgtggccattgaacgtagcggtcatcgttttctgtggtcactgcgtcgtccgacaccgaaagaaaaaattgaatttccgaaagaatatgagaacctggaagaagtgctgccggaaggttttctgaaacgtaccagcagcattggtaaagttattggttgggcaccgcagatggcagttctgagccatccgagcgttggtggttttgttagccattgtggttggaatagcaccctggaaagcatgtggtgtggtgttccgatggcagcatggcctctgtatgcagaacagaccctgaatgcatttctgctggttgttgaattaggtctggcagccgaaattcgtatggattatcgtaccgataccaaagcaggctatgatggtggtatggaagttaccgttgaagaaattgaagatggcattcgcaaactgatgtcagatggtgaaattcgcaacaaagtgaaggacgtgaaagagaaaagtcgcgcagcagttgttgaaggtggttcaagctatgcaagtatcggcaaattcatcgaacatgttagcaacgtgaccatttaa

SEQ ID NO: 8 - protein, 474 aa, synthetic construct
MSTSELVFIPSPGAGHLPPTVELAKLLLHRDQRLSVTIIVMNLWLGPKHNTEARPCVPSLRFVDIPCDESTMALISPNTFISAFVEHHKPRVRDIVRGIIESDSVRLAGFVLDMFCMPMSDVANEFGVPSYNYFTSGAATLGLMFHLQWKRDHEGYDATELKNSDTELSVPSYVNPVPAKVLPEVVLDKEGGSKMFLDLAERIRESKGIIVNSCQAIERHALEYLSSNNNGIPPVFPVGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 9 - DNA, 1473 bp, synthetic construct
  source 1..1473: mol_type = other DNA
atgggcaaacaagaagatgccgaactggttattattccgtttccgtttagcggtcatattctggcaaccattgaactggcaaaacgtctgattagccaggataatccgcgtattcataccattaccattctgtattggggtctgccgtttattccgcaggcagataccattgcatttctgcgtagcctggttaaaaatgaaccgcgtatccgtctggttaccctgccggaagttcaggatccgcctccgatggaactgtttgttgaatttgcagaaagctatatcctggaatatgtgaaaaaaatggtgccgattattcgtgaagcactgagcaccctgctgagcagccgtgatgaaagcggtagcgttcgtgttgcaggtctggttctggattttttctgtgttccgatgattgatgtgggcaacgaatttaatctgccgagctatatctttctgacctgtagcgcaggttttctgggtatgatgaaatatctgccggaacgtcatcgtgaaatcaaaagcgaatttaaccgcagctttaacgaagaactgaatctgattccgggttatgttaatagcgttccgaccaaagtgctgccgagcggtctgtttatgaaagaaacctatgaaccgtgggtagaactggccgaacgttttccggaagcaaaaggtattctggttaatagctataccgcactggaaccgaatggcttcaaatatttcgatcgttgtccggataactacccgaccatttatccgattggtccgattctgaatctggaaaacaaaaaagatgatgccaaaaccgatgaaattatgcgctggctgaatgaacagccggaaagcagcgttgtgtttctgtgctttggtagcatgggtagctttaatgaaaaacaggtgaaagaaattgccgtggcaattgaacgtagtggtcatcgttttctgtggtcactgcgtcgtccgacaccgaaagaaaaaattgaatttccgaaagaatatgagaacctggaagaagttctgcctgaaggctttctgaaacgtaccagcagcattggtaaagttattggttgggcaccgcagatggcagttctgagccatccgagcgttggtggttttgttagccattgtggttggaatagcaccctggaaagcatgtggtgtggtgtgccgatggcagcatggcctctgtatgcagaacagaccctgaatgcctttctgctggttgttgaactgggtttagcagcagaaattcgtatggattatcgtaccgataccaaagccggttatgatggtggtatggaagttaccgttgaagaaattgaagatggcattcgcaaactgatgagtgatggtgaaattcgcaacaaagtgaaggatgtcaaagaaaaatcacgtgcagcagttgttgaaggtggtagcagctatgcaagtattggcaaattcattgaacatgtgagcaacgtgaccatttaa

SEQ ID NO: 10 - protein, 490 aa, synthetic construct
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 11 - DNA, 1425 bp, synthetic construct
  source 1..1425: mol_type = other DNA
atgggcaaacaagaagatgccgaactggttattattccgtttccgtttagcggtcatattctggcaaccattgaactggcaaaacgtctgattagccaggataatccgcgtattcataccattaccattctgtattggggtctgccgtttattccgcaggcagataccattgcatttctgcgtagcctggttaaaaatgaaccgcgtatccgtctggttaccctgccggaagttcaggatccgcctccgatggaactgtttgttgaatttgcagaaagctatatcctggaatatgtgaaaaaaatggtgccgattattcgtgaagcactgagcaccctgctgagcagccgtgatgaaagcggtagcgttcgtgttgcaggtctggttctggattttttctgtgttccgatgattgatgtgggcaacgaatttaatctgccgagctatatctttctgacctgtagcgcaggttttctgggtatgatgaaatatctgccggaacgtcatcgtgaaatcaaaagcgaatttaaccgcagctttaacgaagaactgaatctgattccgggttatgttaatagcgttccgaccaaagtgctgccgagcggtctgtttatgaaagaaacctatgaaccgtgggtagaactggccgaacgttttccggaagcaaaaggtattctggttaatagctataccgcactggaaccgaatggcttcaaatatttcgatcgttgtccggataactacccgaccatttatccgattggtccgattctgtgtagcaatgatcgtccgaatctggatctgagcgaacgtgatcgtattctgaaatggctggatgatcagccggaaagcagcgttgtgtttctgtgctttggtagcctgaaaagcctggcagcaagccagattaaagaaattgcacaggccctggaactggttggtattcgttttctgtggtcaattcgtaccgatccgaaagaatatgcaagcccgaacgaaatcctgccggatggttttatgaatcgtgttatgggtctgggtttagtttgtggttgggcaccgcaggttgaaattctggcacataaagcaattggtggttttgttagccattgcggttggaatagcattctggaaagcctgcgttttggtgtgccgattgcaacctggccgatgtatgcagaacagcagctgaatgcatttaccattgtgaaagaattaggtctggcactggaaatgcgtctggattatgttagcgaatatggcgaaattgtcaaagccgatgaaattgccggtgcagttcgtagcctgatggatggtgaagatgttccgcgtcgtaaactgaaagaaatcgcagaagcaggtaaagaagcagttatggatggcggtagcagctttgttgcagttaaacgttttattgatggcctgtaa

SEQ ID NO: 12 - protein, 474 aa, synthetic construct
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILCSNDRPNLDLSERDRILKWLDDQPESSVVFLCFGSLKSLAASQIKEIAQALELVGIRFLWSIRTDPKEYASPNEILPDGFMNRVMGLGLVCGWAPQVEILAHKAIGGFVSHCGWNSILESLRFGVPIATWPMYAEQQLNAFTIVKELGLALEMRLDYVSEYGEIVKADEIAGAVRSLMDGEDVPRRKLKEIAEAGKEAVMDGGSSFVAVKRFIDGL

SEQ ID NO: 13 - DNA, 1491 bp, synthetic construct
  source 1..1491: mol_type = other DNA
atgggtagccagcaagaacatattgttatgctgccgtttatggcacagggtcatattattccgtttctggcactggcaaaacaaattcagcagcgtaccggttttaccattaccattgcaaatacaccgctgaacattcagtatctgcgtaccaccattagcaccagtgatgatagcagccgtccgtgtattcgtctggcagaactgccgttttgtagcagcgatcatggtctgcctccgaataccgaaaatacagaagcactgagctttcatcagattgtggacctgtttcatgcaagcaaaaccctgcaggcaccgtttcatagcctggttagcggtattattgaaaaagagggtcgtccgcctctgtgcattattagtgatgttttttttggttgggcaaccgaagttgcaaaaagcctgggcaccagcaatgttacctttaccacaggtggtggttatggcaccgcagcatatattagcctgtggcaaaatctgccgcatcgtgcaaccgatagcgattattttgcactgcctggttttccggatagctgtcgttttcatattacccagctgcatcagtacctgcgtgcagcagatggcaccgatgcatggtcacgttattttcagccgcagattgcactgagcctggatagcagcggttggctgtgtaatacagccgaagaaattgaaccgcatggtctggaaattctgcgcaattatgttaaaccgcctgtttggaccattggtccgctgctgcctccggcactgctgaatcatagtctgagcagcggtagcagcatttttggtcagcgtgcatggaaagttagcggtgttagtccggaaaaatgtctggattggctggataaacatccgcagagcagcgttctgtatattagctttggtagccagaataccattagtccgagccagatgatggaactggcattaggtctggaagatagcggtaaaccgtttatttgggttattcgtcctccggtgggttttgatattgaaggtgaatttcgtgcagaatggctgccgcagaactttgaacagcgtatggcagaaagcaatcagggtctgattgttcataaatgggctccgcagctggaaatcctgagccataaaagcaccggtgtgtttctgagccattgtggttggaatagcgttatggaaagcctgtgtgttggtgttccgattattggttggcctctggcagcagaacagtgttataatagcaaaatgctgaccgaagatatgggtgttgcagttgaactgacccgtggtcgtcagggtgcactggaacgtaaagaagttaaacgcgttattgaactggtgatggatagcaaaggtaaaggcgaagagatgaaaaaaaaggccaccgaaattggcgaaaaaattcgtgatgcaatgcgtgaaggtggtagcagcctgaaagcaatggatgattttgttagcaccatgctgagcaaacgtcagggttattaa

SEQ ID NO: 14 - protein, 496 aa, synthetic construct
MGSQQEHIVMLPFMAQGHIIPFLALAKQIQQRTGFTITIANTPLNIQYLRTTISTSDDSSRPCIRLAELPFCSSDHGLPPNTENTEALSFHQIVDLFHASKTLQAPFHSLVSGIIEKEGRPPLCIISDVFFGWATEVAKSLGTSNVTFTTGGGYGTAAYISLWQNLPHRATDSDYFALPGFPDSCRFHITQLHQYLRAADGTDAWSRYFQPQIALSLDSSGWLCNTAEEIEPHGLEILRNYVKPPVWTIGPLLPPALLNHSLSSGSSIFGQRAWKVSGVSPEKCLDWLDKHPQSSVLYISFGSQNTISPSQMMELALGLEDSGKPFIWVIRPPVGFDIEGEFRAEWLPQNFEQRMAESNQGLIVHKWAPQLEILSHKSTGVFLSHCGWNSVMESLCVGVPIIGWPLAAEQCYNSKMLTEDMGVAVELTRGRQGALERKEVKRVIELVMDSKGKGEEMKKKATEIGEKIRDAMREGGSSLKAMDDFVSTMLSKRQGY

SEQ ID NO: 15 - DNA, 1458 bp, synthetic construct
  source 1..1458: mol_type = other DNA
atgggtagcctgggtcatcagctgcatatcttttttctgccgttttttgcacatggccatatgattccgagcgttgatatggcaaaactgtttgcaagccgtggtattaaaaccaccattattaccacaccgctgaacgcaccgttttttagcaaaaccattcagaaaaccaaagagctgggcttcgatattaacatcctgaccatcaaatttccggcagcagaagcaggtctgccggaaggttatgaaaataccgatgcatttatcttcagcgaaaatgcacgtgagatgacgatcaaattcattaaagcaaccacctttctgcaggcaccgtttgaaaaagttctgcaagaatgtcatccggattgtattgttgccgatgttttttttccgtgggcaaccgatgcagcagcaaaatttggtattccgcgtctggtttttcatggcaccagcaattttgcactgagcgcaagcgaatgtgttcgtctgtatgaaccgcataaaaaagttagcagcgatagcgaaccgtttgttgttccggatctgcctggtgatattaaactgaccaaaaaacagctgccggatgatgttcgtgaaaatgtggaaaatgacttcagcaaattcctgaaagcaagcaaagaagcagaactgcgtagctttggtgttgttgtgaatagcttttatgaactggaaccggcatatgcggactactacaaaaaagtgctgggtcgtcgtgcatggaatgttggtccggttagcctgtgtaatcgtgataccgaagataaagcaggtcgtggtaaagaaaccagcattgatcatcatgaatgtctgaaatggctggacagcaaaaaaccgaatagcgttgtgtatatttgctttggtagcaccacgaattttagcgatagccagctgaaagaaattgcagccggtctggaagcaagcggtcagcagtttatttgggttgttcgtcgtaacaaaaaaggccaagaggataaagaagattggctgcctgaaggctttgaagaacgtatggaaggtgttggtctgattattcgtggttgggcaccgcaggttctgattctggatcatgaagcaattggtgcatttgttacccattgtggttggaatagcaccctggaaggtattaccgcaggtaaaccgatggttacctggccgatttttgcagaacagttctataatgaaaaactggtgaccgatgtgctgaaaaccggtgttggtgtgggtgttaaagaatggtttcgtgttcatggtgatcacgttaaaagcgaagcagtggaaaaaaccattacgcagattatggttggtgaagaggccgaagaaatgcgtagccgtgccaaaaaactgggtgaaaccgcacgtaaagcagttgaagaaggtggtagcagctatagtgattttaatgccctgattgaagaactgcgctggcgtcgtccgtaa

SEQ ID NO: 16 - protein, 485 aa, synthetic construct
MGSLGHQLHIFFLPFFAHGHMIPSVDMAKLFASRGIKTTIITTPLNAPFFSKTIQKTKELGFDINILTIKFPAAEAGLPEGYENTDAFIFSENAREMTIKFIKATTFLQAPFEKVLQECHPDCIVADVFFPWATDAAAKFGIPRLVFHGTSNFALSASECVRLYEPHKKVSSDSEPFVVPDLPGDIKLTKKQLPDDVRENVENDFSKFLKASKEAELRSFGVVVNSFYELEPAYADYYKKVLGRRAWNVGPVSLCNRDTEDKAGRGKETSIDHHECLKWLDSKKPNSVVYICFGSTTNFSDSQLKEIAAGLEASGQQFIWVVRRNKKGQEDKEDWLPEGFEERMEGVGLIIRGWAPQVLILDHEAIGAFVTHCGWNSTLEGITAGKPMVTWPIFAEQFYNEKLVTDVLKTGVGVGVKEWFRVHGDHVKSEAVEKTITQIMVGEEAEEMRSRAKKLGETARKAVEEGGSSYSDFNALIEELRWRRP

SEQ ID NO: 17 - DNA, 1395 bp, synthetic construct
  source 1..1395: mol_type = other DNA
atgaaaaaaagcgagctggtgtttattccggcacctggtattggtcatctggttagcgcactggaatttgcaaaacgtctgattgatcatgatgatcgccatagcattaccgttctgagcatgaaatttccgtttacaccgtttgcagatgagtttaccaaaagcctggttgcaagccatcatggtattcaggttattgatctgcctccggttgatccgcctcctccggaactgctgaatagtccggaaaattttgtttgcgcctttatcgaaagcctgattccgcatgttcgtaataccctgaccgatatgctgagcagcagcagccgtgttagcggtctggttctggattttttctgtctgagcctgattgatgttggtaatgaactgggtgttccgagctatatgtttctgaccagcaatagcggttttctgggtctgatgctgcatattccgattcgtcataatcagaaaagcgcagaatttgaagaaagtgatgccgaagttagcctgcctggtttttggaatccggttccggttagcgttctgcctggtgcagtttttgataaagatggtggttatagcgcctatctgaaactggccaaacgttttcgtgataccaaaggcattattgtgaacacctttagtgaactggaactggatagcgttaaaagcctgagtgatggtgaaacccctccggtttatatggttggtccggtgattgatctgaaaggtcagccgaatccgagcctggatcaggcacagcgtgaaaaaatcatgcattggctggatgatcagccggaaagcagcgttgtttatctgtgttttggtagcatgggcacctttggtgcaattcagctgcgtgaaattgcaaccggtctggaacgtagcggtcatcgttttctgtggtcaatgcgtgttccgcagcctgcaaaaggtccggatgatattctgccggaaggttttagcgaacgtattggtggtcgtggtctgatttgtggttgggcaccgcagatggaagttctggcacataaagcaattaaaggctttgttagccattgcggctggaatagcattctggaaagcctgtggtttggtgtgccgattgcaacctggccgatttatgcagaacagcagatgaatgcatttcgcatggttaaagaatttggtctggccgttgaaatgaaactggattatcgtctgggtgatctggttatggcagatgaaattgaaattgccgttcgtcgtctgatggatgatagtgatgaactgcgtaaaaaagtgaaagaaatgggtgaaatggcacgccaggcaattattgaaggtggtagcagctatattgcagttggtcagctgattgaagatattctgggtagcattagctgcgacaactaa

SEQ ID NO: 18 - protein, 464 aa, synthetic construct
MKKSELVFIPAPGIGHLVSALEFAKRLIDHDDRHSITVLSMKFPFTPFADEFTKSLVASHHGIQVIDLPPVDPPPPELLNSPENFVCAFIESLIPHVRNTLTDMLSSSSRVSGLVLDFFCLSLIDVGNELGVPSYMFLTSNSGFLGLMLHIPIRHNQKSAEFEESDAEVSLPGFWNPVPVSVLPGAVFDKDGGYSAYLKLAKRFRDTKGIIVNTFSELELDSVKSLSDGETPPVYMVGPVIDLKGQPNPSLDQAQREKIMHWLDDQPESSVVYLCFGSMGTFGAIQLREIATGLERSGHRFLWSMRVPQPAKGPDDILPEGFSERIGGRGLICGWAPQMEVLAHKAIKGFVSHCGWNSILESLWFGVPIATWPIYAEQQMNAFRMVKEFGLAVEMKLDYRLGDLVMADEIEIAVRRLMDDSDELRKKVKEMGEMARQAIIEGGSSYIAVGQLIEDILGSISCDN

SEQ ID NO: 19 - DNA, 1437 bp, synthetic construct
  source 1..1437: mol_type = other DNA
atggcagaaaccgatagtccgcctcatgttgcaattctgccgagtcctggtatgggtcatctgattccgctggttgaactggcaaaacgtctggttcatcagcataatctgagcgtgacctttattatcccgaccgatggtagcccgagcaaagcacagcgtagcgttctgggtagcctgccgagcaccattcatagcgtttttctgcctccggttaatctgagtgatctgccggaagatgttaaaattgaaaccctgattagcctgaccgttgcacgttcactgccgagcctgcgtgatgttctgagcagcctggttgcaagcggcacccgtgttgttgcactggttgttgacctgtttggcaccgatgcatttgatgttgcacgtgaatttaaagcaagcccgtatatcttttatccggcaccggcaatggcactgagcctgtttttctatctgccgaaactggatgaaatggtgagctgtgaatatagcgaaatgcaagaaccggttgaaattccgggttgtctgccgattcatggtggtgaactgctggatccgacacgtgatcgtaaaaatgatgcatataaatggctgctgcatcacagcaaacgttatcgtctggccgaaggtgttatggtgaatagctttattgatctggaacgtggtgcactgaaagcactgcaagaagttgaaccgggtaaaccgcctgtttatccggttggtccgctggtgaatatggatagcaataccagcggtgttgaaggtagcgaatgtctgaaatggctggatgatcagccgctgggtagcgtgctgtttgttagctttggtagcggtggcaccctgagctttgatcagattaccgaactggcactgggtttagaaatgagcgaacagcgttttctgtgggttgcccgtgttccgaatgataaagttgcaaatgcaacctatttcagcgtggataatcacaaagatccgtttgattttctgccgaagggttttctggatcgtaccaaaggtcgtggtctggttgttccgagctgggcaccgcaggcacaggttctgagccatggtagcaccggtggttttctgacccattgtggttggaatagcaccctggaaagcgttgttaatgcagttccgctgattgtttggcctctgtatgcagaacagaaaatgaatgcatggatgctgaccaaagatgttgaagttgcactgcgtccgaaagcaagcgaaaatggtctgattggtcgtgaagaaattgccaatattgtgcgtggtctgatggaaggtgaagaaggtaaacgcgttcgtaatcgtatgaaagatctgaaagatgcagccgcagaagttctgagcgaagcaggtagcagcaccaaagcactgagtgaagttgcccgtaaatggaaaaaccataaatgtacccaggactgcaactaa

SEQ ID NO: 20 - protein, 478 aa, synthetic construct
MAETDSPPHVAILPSPGMGHLIPLVELAKRLVHQHNLSVTFIIPTDGSPSKAQRSVLGSLPSTIHSVFLPPVNLSDLPEDVKIETLISLTVARSLPSLRDVLSSLVASGTRVVALVVDLFGTDAFDVAREFKASPYIFYPAPAMALSLFFYLPKLDEMVSCEYSEMQEPVEIPGCLPIHGGELLDPTRDRKNDAYKWLLHHSKRYRLAEGVMVNSFIDLERGALKALQEVEPGKPPVYPVGPLVNMDSNTSGVEGSECLKWLDDQPLGSVLFVSFGSGGTLSFDQITELALGLEMSEQRFLWVARVPNDKVANATYFSVDNHKDPFDFLPKGFLDRTKGRGLVVPSWAPQAQVLSHGSTGGFLTHCGWNSTLESVVNAVPLIVWPLYAEQKMNAWMLTKDVEVALRPKASENGLIGREEIANIVRGLMEGEEGKRVRNRMKDLKDAAAEVLSEAGSSTKALSEVARKWKNHKCTQDCN

SEQ ID NO: 21 - DNA, 1455 bp, synthetic construct
  source 1..1455: mol_type = other DNA
atgaccggtgaactgatttttatcccgatgccgagcctgagccatattgcaagcaccatggaaattgcaaaactgctggttcatcgtgatgatcgtctgagcattaccgttctgctgattagcagccagtataccacctcaattaccacctatattaacagcctgattgccagcagcgattatgatcgtattcgttttattcatctgccggaactggatagcgaagaagaaccgaaacgtccgtttatgagcgtgattgatgataacaaaccgatcgttaaagaagccgttaccaatctggcactgagctttgatccgagccatcgtctggcaggttttgttattgatatgttttgcgtgggcatgattgaagttgcagatgaactgggtctgccgagctatccgttttttaccagcagcaccagctttctggccctgcagtttcatgttcagaccctggccgatgaagaagaagttgatattaccgagtttaagaactccgatgttatgctgccgattcctggtctggttaatccgctgcctgcaaaaaccattctgccgagtgcaatgctgaataaagattggctgccgtatgttctgaatggtgcacgtggttttcgtaaaacgaaaggcattatggttaacagctttgccgaaattgaaagcaatgcagttaccagcctgagcaatagcaccgttccgcctgtttataccgttggtccgattattaactttaaaggtgatggtcaggatagcgatacctgtaccgcacacaaatatagcaatattatgacctggctggatgatcagcctccgagcagcgttctgtttctgtgttttggtagcctgggtagctttgatgaagaacaggttaaagaaattgcacgtgccctggaaggtagcggtcatcgttttctgtggtcactgcgtcgtccgcctccgaaagataaaaccatgagctttccgaccgaatatgaaaactttgaagaagtgctgccggaaggttttgtggatcgcaccgttggtatgggtaaagttatgggttgggcaccgcaggttgcagttctggcacatccgagcattggtggttttgtgacccattgtggttggaatagcattctggaaagcgtttggtttggtgttccgatggcagcatggcctctgtatgcagaacagcagtttaatgcatttcatatggtggtggaactgggtttagcagtggaaatcaaaatggattatcgcaaagattatgccattctgggcctgcaagaagaacgcgttagcgcagaagttattgaaaaaggtattcgttgtctgatggaagaggataatgatgcccgtaaaaaagtgaaagaaatgagcgaaattagccgcaaagcactgatggatggtggtagcagccatgccgttctgggtcagtttattgaagatgtgatgaataacatcagcgcctaa

SEQ ID NO: 22 - protein, 484 aa, synthetic construct
MTGELIFIPMPSLSHIASTMEIAKLLVHRDDRLSITVLLISSQYTTSITTYINSLIASSDYDRIRFIHLPELDSEEEPKRPFMSVIDDNKPIVKEAVTNLALSFDPSHRLAGFVIDMFCVGMIEVADELGLPSYPFFTSSTSFLALQFHVQTLADEEEVDITEFKNSDVMLPIPGLVNPLPAKTILPSAMLNKDWLPYVLNGARGFRKTKGIMVNSFAEIESNAVTSLSNSTVPPVYTVGPIINFKGDGQDSDTCTAHKYSNIMTWLDDQPPSSVLFLCFGSLGSFDEEQVKEIARALEGSGHRFLWSLRRPPPKDKTMSFPTEYENFEEVLPEGFVDRTVGMGKVMGWAPQVAVLAHPSIGGFVTHCGWNSILESVWFGVPMAAWPLYAEQQFNAFHMVVELGLAVEIKMDYRKDYAILGLQEERVSAEVIEKGIRCLMEEDNDARKKVKEMSEISRKALMDGGSSHAVLGQFIEDVMNNISA

SEQ ID NO: 23 - DNA, 1470 bp, synthetic construct
  source 1..1470: mol_type = other DNA
atgaaagaaaaagccgaactggtgtttattccgagtccgagcgttggtcatctggttagcgttattgtttttgccaaacgtctgctgctgctggatcgtgataatgcagcaggtagcaataattcctttagcattaccgttctggtgatgcaagaacataccgcaaccgcaagcgataatgatgcacatatcaaaagcctggctggtgccggtgcaaccgcagatgttagcattcgttttattggtgtgccgaaaatgaatccgcctccgctggattatttcaaaagtccggaaaaattcatcaccgagtatgtggatagccacaaagattgtattaaagaggccattattgaacacgtgctgaacaacaatgttaaaattgcaggtctggtgctggattttttctgtagcagcatgattgataccgcaaatgaactgggtgttccgagctatgtgttttttaccagcggtgcagcatttctgggttttgttctgcatctgccgacacgtggtggtgaagaatttgaagaaagcgaagcagatagcgcagatatctttacctatgcaaatccggttccgtatcgtgttctgccgagcctgtgttttaacaaacatggtggttttagcgcctttgaaaattttggtcgtcgcttcaaagaaaccaaaggcattattgtgaacaccttcgaagaactggaaagccatgcagttgaatacctgatgaaatgtgatggtgttccgcctgtttataatgttggtccgatgattgatctgcatggtgatattcatgcacgtccgcgtggcggtggcacccagcgtgatgaaattattcgttggctggatgatcagcctgcaagcagcgttgtttttctgtgttttggtagcatgggtagctttggcgaagaacaaattaaagaagttgcaagcggtctgggtaaaagcggtgttcgttttctgtggtcactgcgtaaaacccctccgaaagatcgtccggaaatgcctggtgaatatacctgtgttgaagatattctgcctcgcggttttcaagaacgtagcaaaggtcgtggtatgatttgtggttgggcaccgcagaaagaagttctggcacatagcgcaattggtggctttgttagccattgcggttggaatagcattctggaaagcgtttggtttggcgttccgattgttacctggccgatttatgccgaacagcagattaatgcatttcagatggttcgtgatctgggtttagcagttgaactgcgtctggattatcgtcgtggcaccgaaaatcatgttatggcagatgaactggcacgtgcggttggttgtgttatggatggtgatagcgaagttcgtaaacgtgtgaaagaagtgagcgaaaaagcacgtctggcactgcgtgatggtggtagcagctatgcagcaaccggtcgtctgattgaagatttatttggtagcgtgagctaa

SEQ ID NO: 24 - protein, 489 aa, synthetic construct
MKEKAELVFIPSPSVGHLVSVIVFAKRLLLLDRDNAAGSNNSFSITVLVMQEHTATASDNDAHIKSLAGAGATADVSIRFIGVPKMNPPPLDYFKSPEKFITEYVDSHKDCIKEAIIEHVLNNNVKIAGLVLDFFCSSMIDTANELGVPSYVFFTSGAAFLGFVLHLPTRGGEEFEESEADSADIFTYANPVPYRVLPSLCFNKHGGFSAFENFGRRFKETKGIIVNTFEELESHAVEYLMKCDGVPPVYNVGPMIDLHGDIHARPRGGGTQRDEIIRWLDDQPASSVVFLCFGSMGSFGEEQIKEVASGLGKSGVRFLWSLRKTPPKDRPEMPGEYTCVEDILPRGFQERSKGRGMICGWAPQKEVLAHSAIGGFVSHCGWNSILESVWFGVPIVTWPIYAEQQINAFQMVRDLGLAVELRLDYRRGTENHVMADELARAVGCVMDGDSEVRKRVKEVSEKARLALRDGGSSYAATGRLIEDLFGSVS

SEQ ID NO: 25 - DNA, 1416 bp, synthetic construct
  source 1..1416: mol_type = other DNA
atgaaaaagccggaactgatctttattccggcacctggtgttggtcatgttattagcgcactggaatttgcagcacgtctgctgggtcatgatccgcgtattagcattaccgttctggcaattaaagcaccgtttagcagccatctggatgcatataccaaaagcctggttgcaagccagcctcgtattgcactgattgatctgcctccggttgatccgcctcctccggaactgctgcgtagcgttgaacattatgttaccgtttatatcgagagctatatcccgcatgttcagcgtgcagttggtgaaattcagagcagccgtagcgatagcggtagcgatcgtagcgtggcaggttttgttctggattttttctgtaccagcatgattgatgttgccgatggttttagcctgccgagttatatctatctgaccagcaatgcacatttcctggatctgatgtttcatctgcctgcacgtcatgatcagattggtcgtgaagcccgtctgagcgatcctgatctgccgctgccgaattttgcaaatccggttccggttagcgttctgccgagcgcagttctggatggtgaagccggtggttatgcagcatatgttaaactggcacgtcgttttcgtgatgttcgtggtattatggtgaacacctttatggaactggaacgtagtgcactgggtagctttagcggtggtagctgggatggtccggcagtttatgccgttggtccggttattgatccgaaaggtgtgccggatccgagcctggatcgtgcacattgggaaaaaattcgtgaatggctggatgaacagcctccgagcagcgttgtttttctgtgttttggtagcctgggtagttttgatgccgatcaggttaaagaaattgccagcggtattgaacgtagcggtcataaatttctgtggtcactgcgtccggaaagcagccagagccagctgccggttggttttctggaacggatgcgtggtcgcggtattatttgtggttggacaccgcaggttgaagttctgggccatgcagcaattggtggttttgttagccattgcggttggaatagcattctggaaagcctgtggtatggtgttccgattgttacctggccgatttatgcagaacagcagctgaatgcatttaccatggtgaaagaactgggtttagcagttgaactgcgtctggattacaaaggtcaaggtattggtggtcatctggttccggcagaagaaattgaaaaagcagttggtcgtctgatgggagatgatgcagcagaaacccgtaaaaaagtgcgtgaaatgcgtgaagcagcacgtaatgcagttaaagaaggtggtagcagctacgatagcatcggtcgcctgattcaggattttatcaacagcatcgaataa

SEQ ID NO: 26 - protein, 471 aa, synthetic construct
MKKPELIFIPAPGVGHVISALEFAARLLGHDPRISITVLAIKAPFSSHLDAYTKSLVASQPRIALIDLPPVDPPPPELLRSVEHYVTVYIESYIPHVQRAVGEIQSSRSDSGSDRSVAGFVLDFFCTSMIDVADGFSLPSYIYLTSNAHFLDLMFHLPARHDQIGREARLSDPDLPLPNFANPVPVSVLPSAVLDGEAGGYAAYVKLARRFRDVRGIMVNTFMELERSALGSFSGGSWDGPAVYAVGPVIDPKGVPDPSLDRAHWEKIREWLDEQPPSSVVFLCFGSLGSFDADQVKEIASGIERSGHKFLWSLRPESSQSQLPVGFLERMRGRGIICGWTPQVEVLGHAAIGGFVSHCGWNSILESLWYGVPIVTWPIYAEQQLNAFTMVKELGLAVELRLDYKGQGIGGHLVPAEEIEKAVGRLMGDDAAETRKKVREMREAARNAVKEGGSSYDSIGRLIQDFINSIE

SEQ ID NO: 27 - DNA, 1434 bp, synthetic construct
  source 1..1434: mol_type = other DNA
atgaagaaaatcaacctggtggttattccgtttgccggtgttggtcatctggttagcaccattgaattcattaaacgtctgctgagccgtgatgatcgttttagcgttaccattctggtgattaaaagtccgctggaacatagcctggatccgaataccagtccgcctagcctgccgagcagccatccgcatattcagtttattaacgttcgtagcctgaatccgcctccgattgaagtttttctgaaaagtccggaaaaatactttaccctgtacatcgagagctacaagctgaaagtgaaagaaatcattgtggatcatgttctgccgaatagcgcaccgttaggtggtctgtttctggatatgtttagcaccagcatggttgatgttgcacaagaactgggtgttccgagctatgtgtttctgaccagcggtgcaggttttctgggttttgttctgtatctgcagacccgtcatgatcgcggtggtgcagaatttgaactgagcgatattgaaagctatatcccgagttatgttaatccggttccgagccgtgtgctgccgagcctggcatttaatcgtgaaggtggttatgttagctttgtgaatgttgcccgtaatatgaaaaaaaccaaaggcattattgtgaacaccgtgtttgaactggaaagccatgcaattagcagcctgctgatggatggtgcaacccctccggtttatgcagttggtccgctgattgatcataaaggtgaaagccattttcgtaccgatcaggcaaaacgcgataaaatcattaaatggctggatgatcagccgaccaaaagcgttatttttctgtgctttggttcctttggtagctttggtgttcctcagctgcgtgaaattgccattggtctggaacagagcggtgcccgtttcctgtggtcaattcgtcaggccaaaagcatttttgaaatgccgattgaatgcgagaaaccggaagaatttctgccgcagggctttctggatcgtaccaaagaattaggtatgatttgtggttggacaccgcagattgaagtgctggcacatcgtgcaattggtggttttgtgagccattgcggttggaatagcattctggaaagtctgtggcatggtgtgccgattgttacctggcctctgtatgcagaacagcagattaatgcatttcagatcgtgaaagatttaggcctgggtgttgaactgcgtctggattatcgtcgtgatggcggtgatgttgatgtggttatgcgtgatgaaattgaaaaagccgttcgctatgttatggatggcgatagcgaagttcgtcagcgtgttcaagaggcaagcgaaaaatgtcgtaaagcagttgtggaaggtggtagcagctttagcagcattggtagcctgattgaagcaattctggccaacattagctaa

SEQ ID NO: 28 - protein, 477 aa, synthetic construct
MKKINLVVIPFAGVGHLVSTIEFIKRLLSRDDRFSVTILVIKSPLEHSLDPNTSPPSLPSSHPHIQFINVRSLNPPPIEVFLKSPEKYFTLYIESYKLKVKEIIVDHVLPNSAPLGGLFLDMFSTSMVDVAQELGVPSYVFLTSGAGFLGFVLYLQTRHDRGGAEFELSDIESYIPSYVNPVPSRVLPSLAFNREGGYVSFVNVARNMKKTKGIIVNTVFELESHAISSLLMDGATPPVYAVGPLIDHKGESHFRTDQAKRDKIIKWLDDQPTKSVIFLCFGSFGSFGVPQLREIAIGLEQSGARFLWSIRQAKSIFEMPIECEKPEEFLPQGFLDRTKELGMICGWTPQIEVLAHRAIGGFVSHCGWNSILESLWHGVPIVTWPLYAEQQINAFQIVKDLGLGVELRLDYRRDGGDVDVVMRDEIEKAVRYVMDGDSEVRQRVQEASEKCRKAVVEGGSSFSSIGSLIEAILANIS

(19) United States
(12) Patent Application Publication
(10) Pub. No.: US 2025/0305018 A1
(43) Pub. Date: Oct. 2, 2025

(54) SYNTHESIS OF GLYCOSYLATED HYDROXYTRYPTAMINE COMPOUNDS AND METHODS OF THEIR USE

(71) Applicant: Cy Biopharma AG, Baar (CH)

(72) Inventor: James Morrison, Montevideo (UY)

(21) Appl. No.: 18/864,286

(22) PCT Filed: May 9, 2023
(86) PCT No.: PCT/EP2023/062319

§ 371 Date: Nov. 8, 2024

Related U.S. Application Data
(60) Provisional application No. 63/339,560, filed on May 9, 2022.
(60) Provisional application No. 63/373,693, filed on Aug. 26, 2022.

(51) Int. Cl. C12P 19/60; A61K 31/7056; C12N 9/10; C12P 19/18

(52) U.S. Cl. CPC C12P 19/60; A61K 31/7056; C12N 9/1048; C12P 19/18

(57) ABSTRACT

Provided herein is a method of treating a disorder (e.g. depression, anxiety, pain, inflammation, or addiction), or symptoms thereof, by administering an effective amount of a glycosylated hydroxytryptamine compound, such as compounds of formula (I) or a pharmaceutically acceptable salt thereof, wherein R1 is a glycosyl group and the other variables are as defined herein. Also provided herein is a method of enzymatically synthesizing the glycosylated hydroxytryptamine compounds.

### CROSS-REFERENCE TO RELATED APPLICATIONS

This application claims priority to U.S. Provisional Application No. 63/339,560, filed on May 9, 2022, and to U.S. Provisional Application No. 63/373,693, filed Aug. 26, 2022, which are incorporated by reference herein in their entireties.

### BACKGROUND

New and improved compounds are needed that act as modulating agents at serotonin receptors, as well as agents for the prevention and treatment of diseases and disorders. The compounds, compositions, and methods described herein are directed toward this end.

### SUMMARY

The disclosures features compounds useful for treating diseases, or symptoms thereof, as well as methods for synthesizing compounds useful for treating diseases, or symptoms thereof.

Provided herein are compounds related to hydroxytryptamine compounds, such as 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT, and analogues or derivatives thereof (e.g., glycosylated analogues or derivatives).

Compounds of the disclosure act on the serotonin receptors, such as the 5-HT2A receptor. In some embodiments, such compounds are envisioned to be useful as therapeutic agents for treating depression, anxiety, pain, inflammation, addiction, eating disorders, obsessive compulsive disorders, and autoimmune diseases.

In some embodiments, provided herein is a compound of Formula (I):

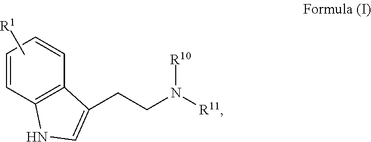

or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a compound of Formula (Ia):

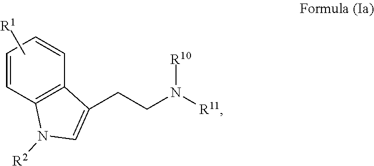

or a pharmaceutically acceptable salt thereof,
wherein
R¹ is a glycosyl group or —OH;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a compound of Formula (I):

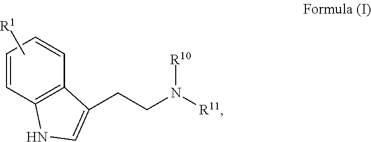

or a pharmaceutically acceptable salt thereof,
wherein
R¹ is an —O-glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a compound of Formula (Ia):

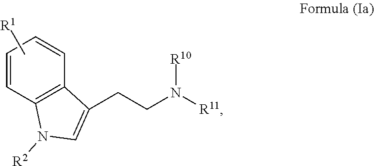

or a pharmaceutically acceptable salt thereof,
wherein
R¹ is an —O-glycosyl group or —OH;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, compounds of the disclosure are formed from glycosylation.

In some embodiments, compounds of the disclosure are formed from glycosylation, using an enzyme.

In some embodiments, a pharmaceutical composition comprises a compound described herein or pharmaceutically acceptable salt thereof, and a pharmaceutically acceptable excipient.

In some embodiments, a method of treating a disease, or symptoms thereof, in a subject in need thereof, comprises administering to the subject an effective amount of a compound described herein or a pharmaceutically acceptable salt thereof.

In some embodiments, the disclosure provides a method of treating a disease selected from depression, anxiety, pain, inflammation, and addiction, and symptoms thereof, in a subject in need thereof, comprises administering to the subject an effective amount of a compound described herein or a pharmaceutically acceptable salt thereof. In some embodiments, the disclosure provides a method of treating a disease selected from an eating disorder, an obsessive compulsive disorder, and an autoimmune disease, and symptoms thereof, in a subject in need thereof, comprises administering to the subject an effective amount of a compound described herein or a pharmaceutically acceptable salt thereof.

In one aspect, provided herein is a pharmaceutically acceptable salt of a compound described herein (e.g., a compound of Formula (I), Formula (Ia), Formula (Ib), Formula (Ibb), Formula (Ic), Formula (Ie), Formula (Ie-α), or Formula (Ie-β), or a subformula thereof).

In one aspect, provided herein is a pharmaceutical composition comprising a compound described herein (e.g., a compound of Formula (I), Formula (Ia), Formula (Ib), Formula (Ibb), Formula (Ic), Formula (Ie), Formula (Ie-α), or Formula (Ie-β), or a subformula thereof) or a pharmaceutically acceptable salt thereof, and a pharmaceutically acceptable excipient. In certain embodiments, the compound of the present invention is provided in an effective amount in the pharmaceutical composition. In certain embodiments, the compound of the present invention is provided in a therapeutically effective amount. In certain embodiments, the compound of the present invention is provided in a prophylactically effective amount.

Compounds of the present invention as described herein, act, in certain embodiments, as serotonin modulators, e.g., effecting the serotinin receptor in either a positive or negative manner.

In some embodiments, provided herein is a method of treating a disease, or symptoms thereof, in a subject in need thereof, comprising administering to the subject an effective amount of a compound of Formula (I):

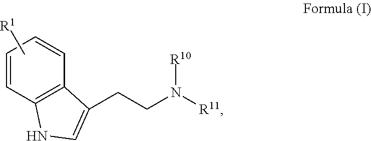

or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ia):

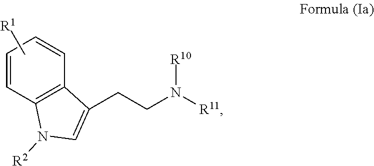

or a pharmaceutically acceptable salt thereof,
wherein
R¹ is a glycosyl group or —OH;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ib):

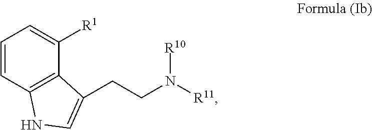

or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (I):

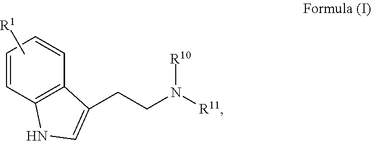

or a pharmaceutically acceptable salt thereof,
wherein
R¹ is an —O-glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ia):

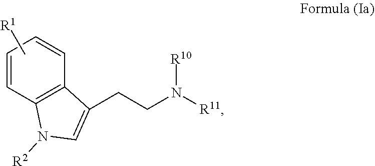

or a pharmaceutically acceptable salt thereof,
wherein
R¹ is an —O-glycosyl group or —OH;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ib):

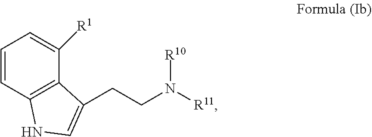

or a pharmaceutically acceptable salt thereof,
wherein
R¹ is an —O-glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, the disease is selected from depression, anxiety, pain, inflammation, addiction, an eating disorder, an obsessive compulsive disorder, and an autoimmune disease, and symptoms thereof. In some embodiments, the disease is selected from depression, anxiety, pain, inflammation, and addiction, and symptoms thereof. In some embodiments, the disease is selected from an eating disorder, an obsessive compulsive disorder, and an autoimmune disease, and symptoms thereof.

In certain embodiments, the compound is administered orally, subcutaneously, intravenously, or intramuscularly. In certain embodiments, the compound is administered orally. In certain embodiments, the compound is administered intravenously. In certain embodiments, the compound is administered chronically. In certain embodiments, the compound is administered continuously, e.g., by continuous intravenous infusion.

In some embodiments, provided herein is a process of preparing a compound of Formula (I):

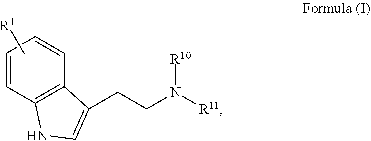

or a salt thereof, comprising:

glycosylating the hydroxy group of a compound of Formula (I-1):

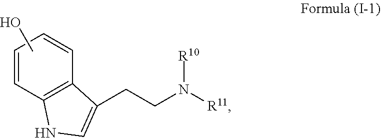

or a salt thereof, thereby producing the compound of Formula (I),
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

### BRIEF DESCRIPTION OF THE DRAWINGS

FIG. 1 illustrates the glycosylation reaction mediated by “retaining” or “inverting” glycosyltransferases (stereochemistry of the donor's anomeric bond is retained (α→α) or inverted (α→β) during the transfer).

FIG. 2 shows the pGLY expression plasmid for GLY70.

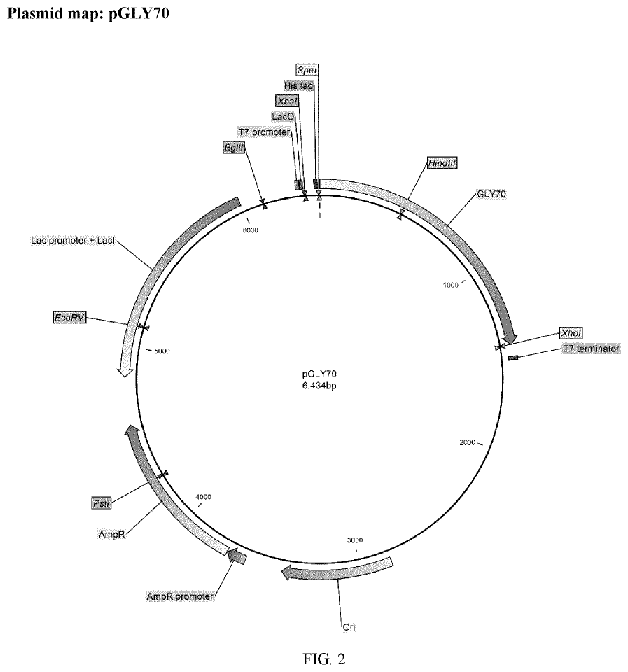

FIG. 3 shows the pGLY expression plasmid for GLY73.

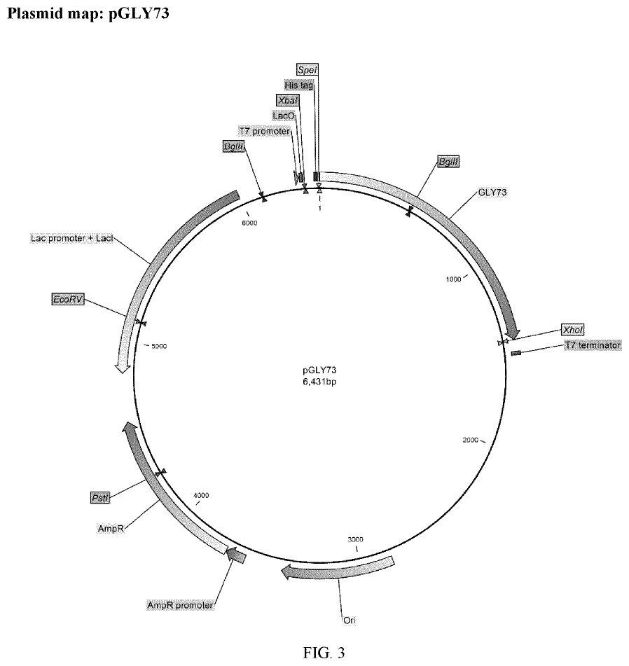

FIG. 4 shows the pGLY expression plasmid for GLY74.

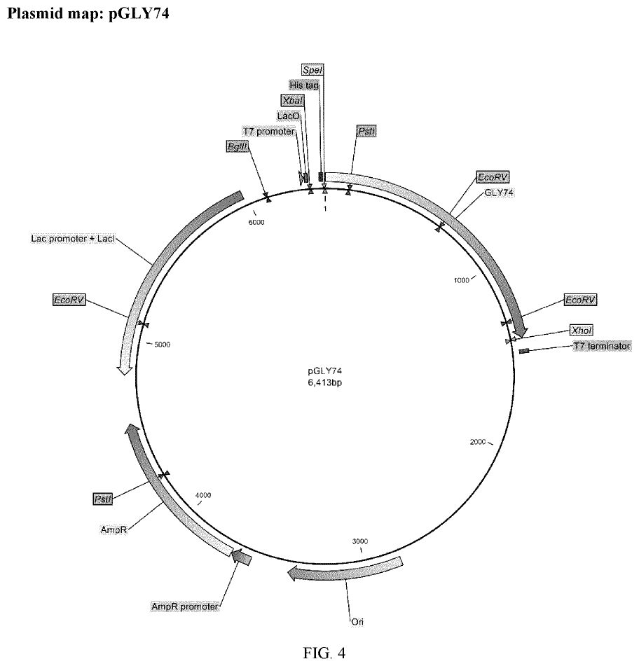

FIG. 5 shows the pGLY expression plasmid for GLY77.

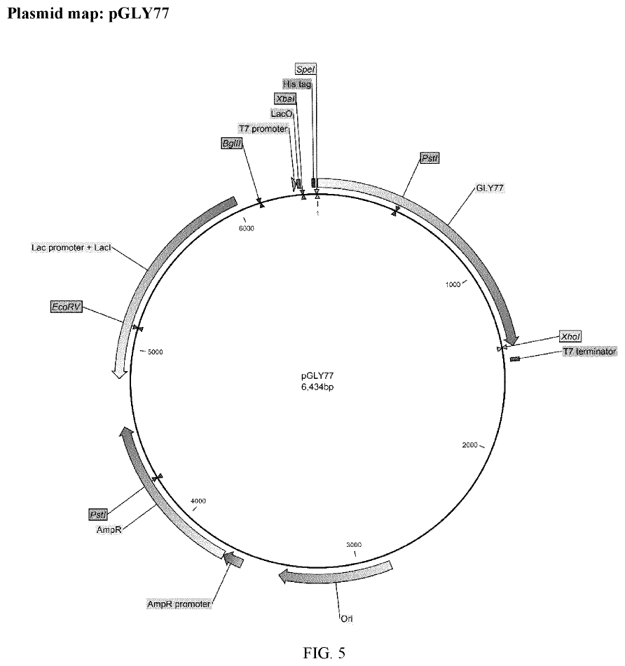

FIG. 6 shows the pGLY expression plasmid for GLY87.

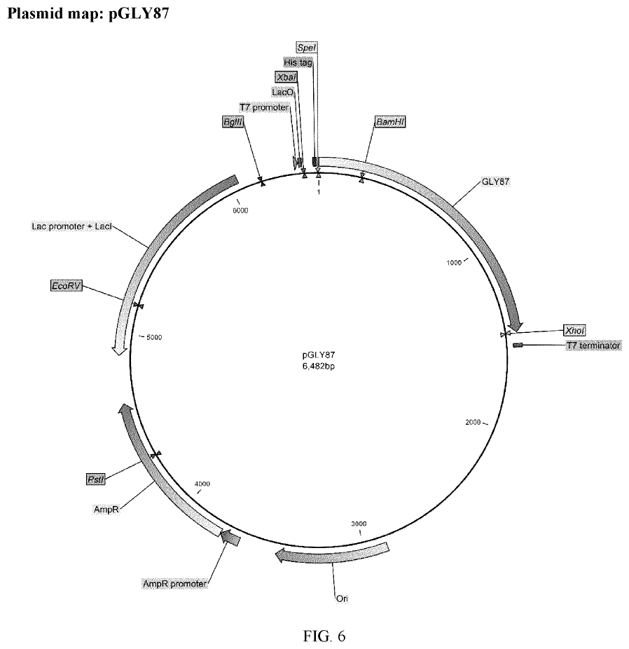

FIG. 7 shows the pGLY expression plasmid for GLY91.

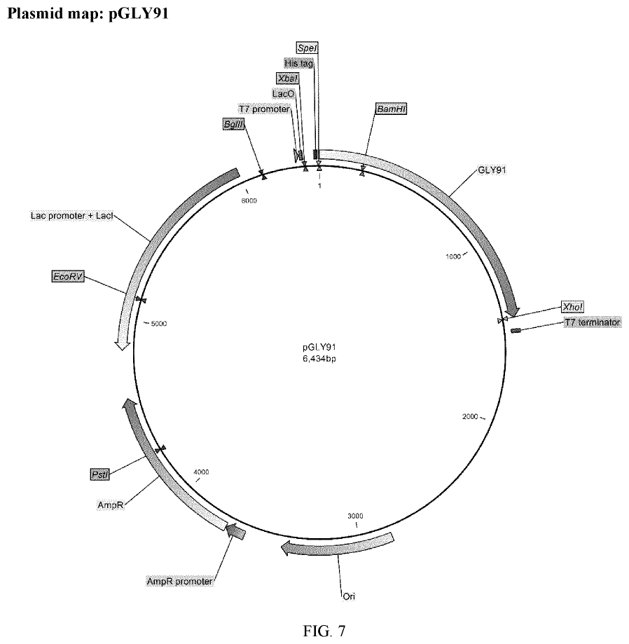

FIG. 8 shows the pGLY expression plasmid for GLY108.

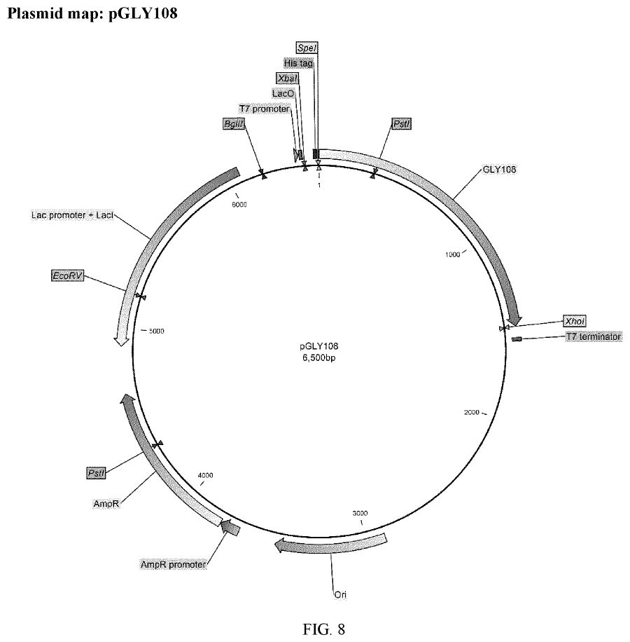

FIG. 9 shows the pGLY expression plasmid for GLY119.

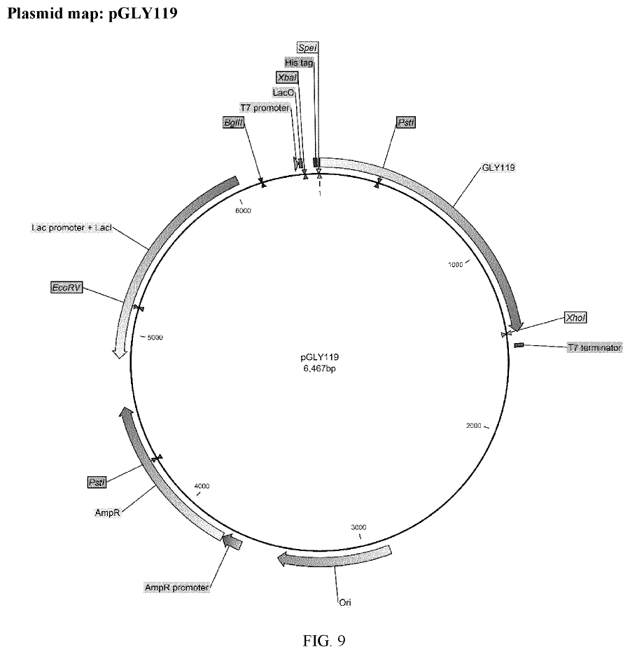

FIG. 10 shows the pGLY expression plasmid for GLY146.

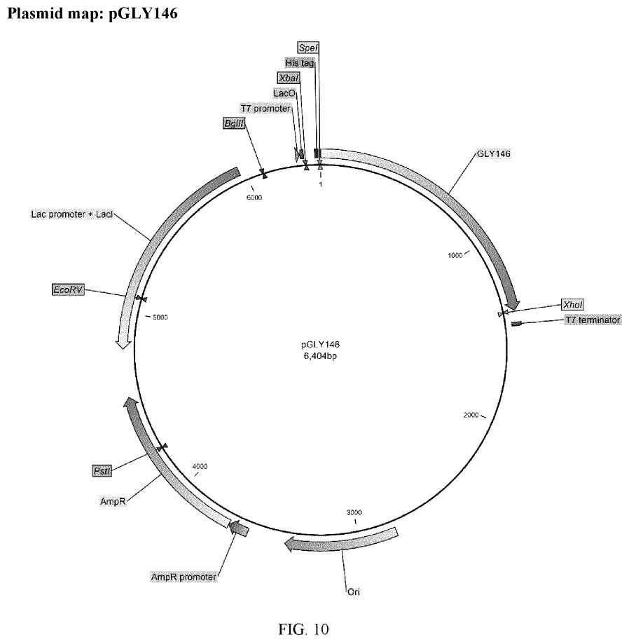

FIG. 11 shows the pGLY expression plasmid for GLY153.

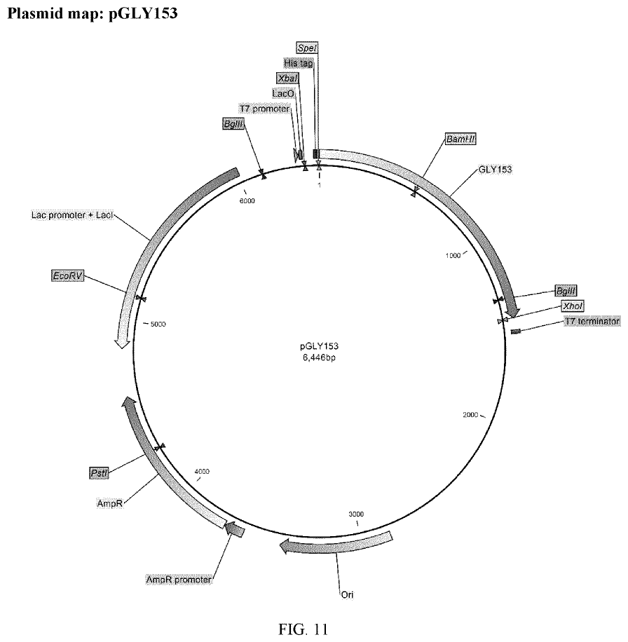

FIG. 12 shows the pGLY expression plasmid for GLY163.

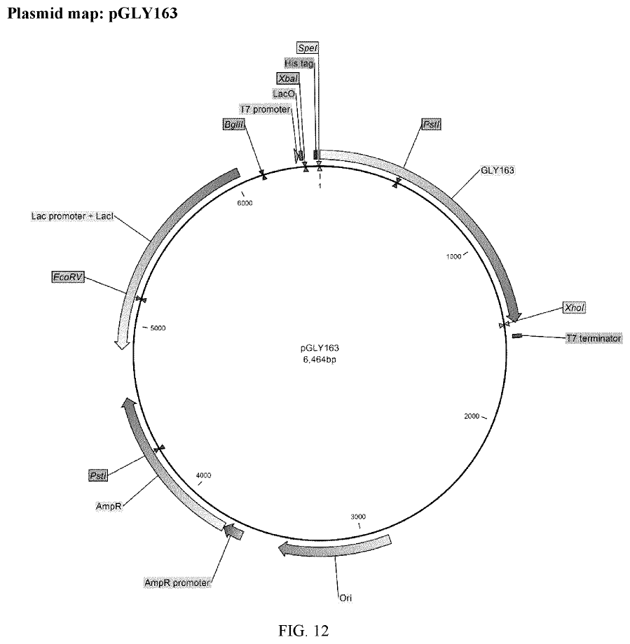

FIG. 13 shows the pGLY expression plasmid for GLY164.

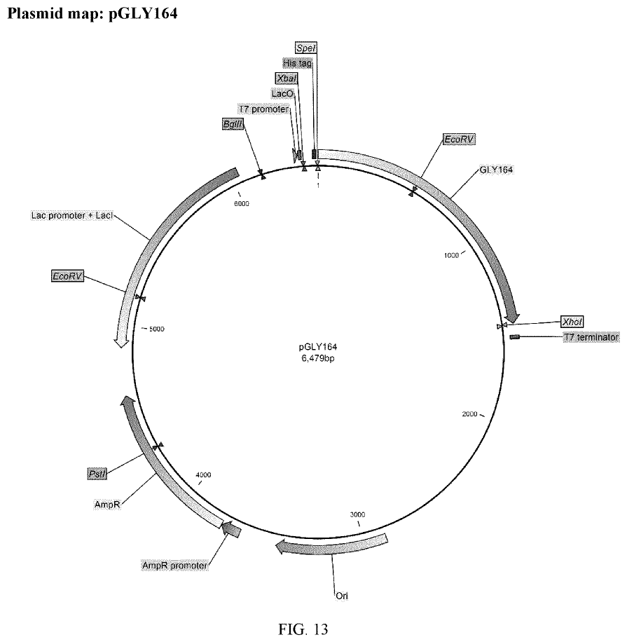

FIG. 14 shows the pGLY expression plasmid for GLY169.

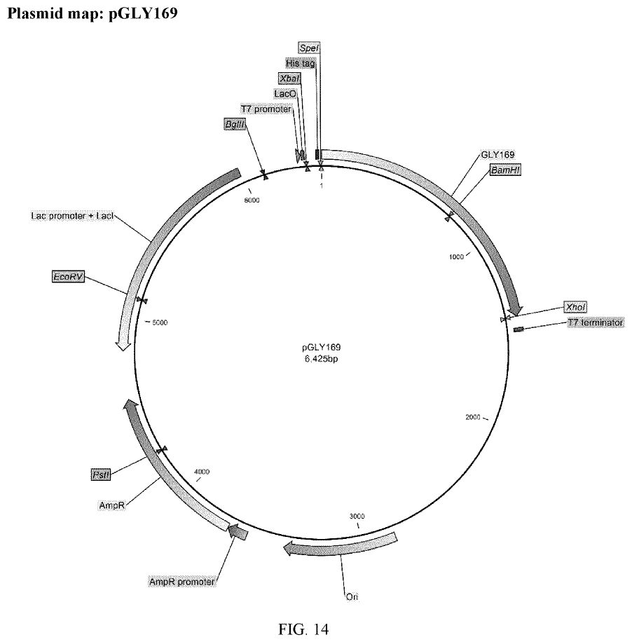

FIG. 15 shows the pGLY expression plasmid for GLY170.

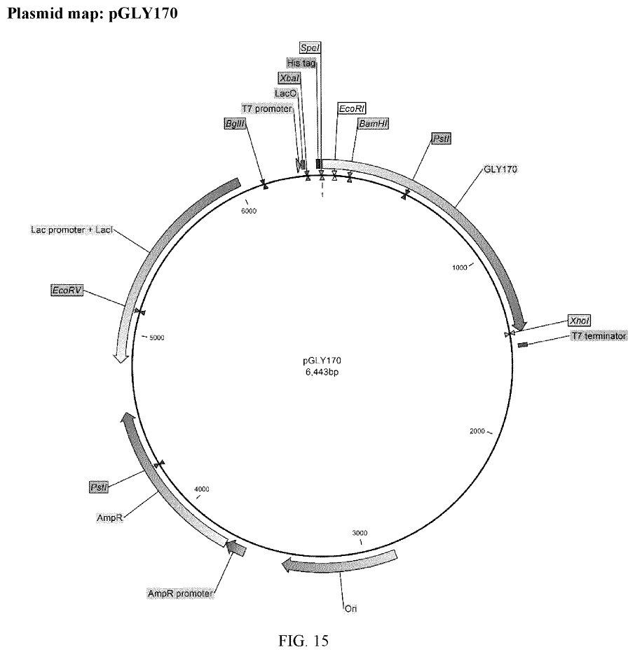

FIG. 16 is a series of chromatograms obtained with a wavelength of 268 nm for the standard solutions resuspended in reaction buffer, and the corresponding MS-SIM signal for 4-HO-MET with no enzyme (control), GLY70, or GLY74.

FIG. 17 is a table showing ¹³C and ¹H chemical shifts, coupling patterns, and coupling constants for ¹H resonance, as well as 2D NMR correlations from COSY, NOESY/ROESY, HSQC, and HMBC for 4-HO-MET glycoside.

FIG. 18 depicts ¹H NMR spectrum of 4-HO-MET glycoside.

FIG. 19 depicts ¹³C NMR spectrum of 4-HO-MET glycoside.

FIG. 20 depicts DQF-COSY spectrum of 4-HO-MET glycoside.

FIG. 21 depicts HSQC spectrum of 4-HO-MET glycoside.

FIG. 22 depicts ROESY spectrum of 4-HO-MET glycoside.

FIG. 23 depicts NOESY spectrum (600 ms mixing time) of 4-HO-MET glycoside.

FIG. 24 depicts NOESY spectrum (600 ms mixing time) of 4-HO-MET glycoside.

FIG. 25 depicts HMBC spectrum of 4-HO-MET glycoside.

FIG. 26 is a table showing ¹³C and ¹H chemical shifts, coupling patterns, and coupling constants for ¹H resonance, as well as 2D NMR correlations from COSY, NOESY/ROESY, HSQC, and HMBC for 4-HO-EPT glycoside.

FIG. 27 depicts ¹H NMR spectrum of 4-HO-EPT glycoside.

FIG. 28 depicts ¹³C NMR spectrum of 4-HO-EPT glycoside.

FIG. 29 depicts QDEPT spectrum of 4-HO-EPT glycoside

FIG. 30 depicts DQF-COSY spectrum of 4-HO-EPT glycoside.

FIG. 31 depicts HSQC spectrum of 4-HO-EPT glycoside.

FIG. 32 depicts ROESY spectrum of 4-HO-EPT glycoside.

FIG. 33 depicts NOESY spectrum (600 ms mixing time) of 4-HO-EPT glycoside.

FIG. 34 depicts HMBC spectrum of 4-HO-EPT glycoside.

FIG. 35 displays pharmacokinetics of Cy1 and 4-OH-EPT in plasma for Cy1 20 mg/kg dosed orally.

FIG. 36 displays pharmacokinetics of Cy1 and 4-OH-EPT in brain for Cy1 20 mg/kg dosed orally.

FIG. 37 displays pharmacokinetics of Cy2 and 4-OH-DMT (psilocin) in plasma for Cy2 20 mg/kg dosed orally.

FIG. 38 displays pharmacokinetics of Cy2 and 4-OH-DMT (psilocin) in brain for Cy2 20 mg/kg dosed orally.

FIG. 39 displays pharmacokinetics of 4-OH-DMT (psilocin) in plasma for psilocybin 1.5 mg/kg dosed orally.

FIG. 40 displays pharmacokinetics of 4-OH-DMT (psilocin) in brain for psilocybin, 1.5 mg/kg dosed orally.

### DETAILED DESCRIPTION

As generally described herein, the present disclosure provides compounds designed, for example, to act as serotonin receptor modulators. The compounds disclosed herein are useful for treating a disease (e.g., anxiety, pain, inflammation, addiction, an eating disorder, an obsessive compulsive disorder, and an autoimmune disease) or symptoms thereof. The present disclosure also features methods for synthesizing compounds useful for treating a disease (e.g., anxiety, pain, inflammation, addiction, an eating disorder, an obsessive compulsive disorder, and an autoimmune disease) or symptoms thereof. In certain embodiments, such compounds are envisioned to be useful as therapeutic agents for treating treating depression, anxiety, pain, inflammation, addiction, an eating disorder, an obsessive compulsive disorder, or an autoimmune disease, or symptoms thereof.

### Definitions

As used herein, the following terms are generally intended to have the meanings as described below, unless expressly indicated otherwise or the context in which they are used indicates otherwise. Unless defined otherwise, the terms used herein have meanings as commonly understood by one of ordinary skill in the areas related to the present disclosure.

### Chemical Definitions

The abbreviations used herein have their conventional meaning within the chemical and biological arts. The chemical structures and formulas set forth herein are constructed according to the standard rules of chemical valency known in the chemical arts.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Isomers, e.g., stereoisomers, can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-position/center/carbon compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

“Alkyl” refers to a radical of a straight-chain or branched saturated hydrocarbon group having from 1 to 20 carbon atoms (“C₁-C₂₀ alkyl”). In some embodiments, an alkyl group has 1 to 12 carbon atoms (“C₁-C₁₂ alkyl”). In some embodiments, an alkyl group has 1 to 10 carbon atoms (“C₁-C₁₀ alkyl”). In some embodiments, an alkyl group has 1 to 9 carbon atoms (“C₁-C₉ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”, also referred to herein as “lower alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (Cs), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

“Alkenyl” refers to a radical of a straight-chain or branched hydrocarbon group having from 2 to 20 carbon atoms, one or more carbon-carbon double bonds, and no triple bonds (“C₂-C₂₀ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl or 1-propenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-4 alkenyl groups as well as pentenyl (C), pentadienyl (Cs), hexenyl (C₆), and the like. Additional examples of alkenyl include heptenyl (C₇), octenyl (C₈), octatrienyl (C₈), and the like. Each instance of an alkenyl group may be unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₆ alkenyl.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the ring system. In some embodiments, a cycloalkyl group has 3 to 8 ring carbon atoms (“C₃-C₈cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. Each instance of a cycloalkyl group may be unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” refers to a radical of a non-aromatic ring system having ring carbon atoms and one or more (e.g., 1 to 4) ring heteroatoms, wherein each heteroatom is independently selected from nitrogen, oxygen, and sulfur. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. A heterocyclyl group may be described as, e.g., a 3- to 10-membered heterocyclyl, wherein the term “membered” refers to the non-hydrogen ring atoms, i.e., carbon, nitrogen, oxygen, and sulfur, within the moiety. Each instance of heterocyclyl may be independently optionally substituted, i.e., unsubstituted or substituted with one or more substituents. In certain embodiments, the heterocyclyl group is unsubstituted 3- to 10-membered heterocyclyl. In certain embodiments, the heterocyclyl group is substituted 3- to 10-membered heterocyclyl.

4-HO-MET or 4-hydroxy-N-methyl-N-ethyltryptamine, having the structure:

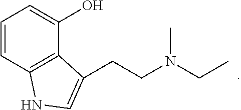 [SMILES: CCN(C)CCc1c[nH]c2cccc(O)c12]

4-HO-MPT or 4-hydroxy-N-methyl-N-propyltryptamine, having the structure:

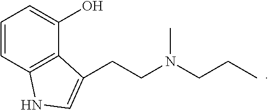 [SMILES: CCCN(C)CCc1c[nH]c2cccc(O)c12]

4-OH-MiPT or 4-hydroxy-N-methyl-N-isopropyltryptamine, having the structure

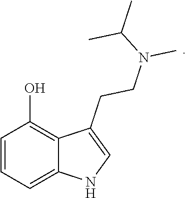 [SMILES: CC(C)N(C)CCc1c[nH]c2cccc(O)c12]

4-HO-McPeT or 4-hydroxy-N-methyl-N-cyclopentyltryptamine, having the structure:

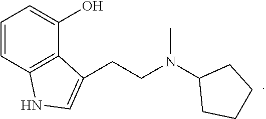 [SMILES: CN(CCc1c[nH]c2cccc(O)c12)C1CCCC1]

4-HO-McPT or 4-hydroxy-N-cyclopropyl-N-methyltryptamine, having the structure:

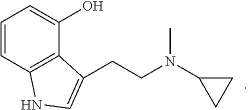 [SMILES: CN(CCc1c[nH]c2cccc(O)c12)C1CC1]

4-HO-MALT or 44-hydroxy-N-methyl-N-allyltryptamine, having the structure:

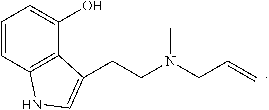 [SMILES: C=CCN(C)CCc1c[nH]c2cccc(O)c12]

4-HO-DALT or 4-hydroxy-N,N-diallyltryptamine, having the structure:

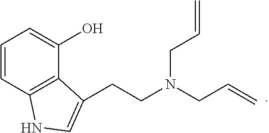 [SMILES: C=CCN(CC=C)CCc1c[nH]c2cccc(O)c12]

4-HO-DET or 4-hydroxy-diethyltryptamine, having the structure:

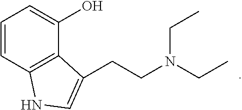 [SMILES: CCN(CC)CCc1c[nH]c2cccc(O)c12]

4-HO-EPT or 4-hydroxy-N-ethyl-N-propyltryptamine, having the structure:

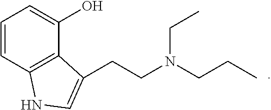 [SMILES: CCCN(CC)CCc1c[nH]c2cccc(O)c12]

4-HO-DPT or 4-hydroxy-N,N-dipropyltryptamine, having the structure:

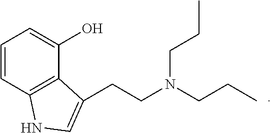 [SMILES: CCCN(CCC)CCc1c[nH]c2cccc(O)c12]

4-HO-PiPT or 4-hydroxy-N-propyl-N-isopropyltryptamine, having the structure:

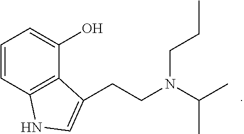 [SMILES: CCCN(CCc1c[nH]c2cccc(O)c12)C(C)C]

4-HO-DiPT or 4-hydroxy-N,N-diisopropyltryptamine, having the structure:

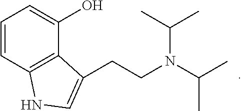 [SMILES: CC(C)N(CCc1c[nH]c2cccc(O)c12)C(C)C]

4-HO-DBT or 4-hydroxy-N,N-dibutyltryptamine, having the structure:

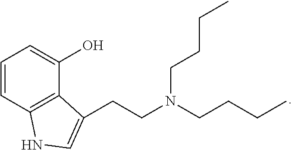 [SMILES: CCCCN(CCCC)CCc1c[nH]c2cccc(O)c12]

4-HO-DSBT or 4-hydroxy-N,N-disecbutyltryptamine, having the structure:

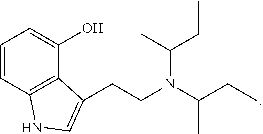 [SMILES: CCC(C)N(CCc1c[nH]c2cccc(O)c12)C(C)CC]

4-HO-pyr-T or 4-hydroxy-N,N-tetramethylenetryptamine, having the structure:

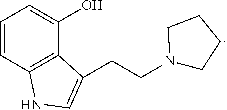 [SMILES: Oc1cccc2[nH]cc(CCN3CCCC3)c12]

### Other Definitions

4-HO-DMT or 4-hydroxy-N,N-dimethyltryptamine or psilocin, having the structure:

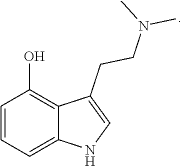

5-HO-DMT or N,N-dimethyl-5-hydroxytryptamine or 5-hydroxy-dimethyltryptamine, having the structure:

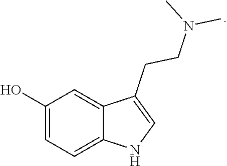

As used herein, the term “modulation” refers to the inhibition or potentiation of serotonin receptor function. A “modulator” (e.g., a modulator compound) may be, for example, an agonist, partial agonist, antagonist, or partial antagonist of the serotonin receptor.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

“Solvate” refers to forms of the compound that are associated with a solvent or water (also referred to as “hydrate”), usually by a solvolysis reaction. This physical association includes hydrogen bonding. Conventional solvents include water, ethanol, acetic acid, and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of x electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject Is a non-human animal. The terms “human,” “patient,” and “subject” are used interchangeably herein.

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference. Exemplary oxygen protecting groups include, but are not limited to, methyl, methoxylmethyl (MOM), 2-methoxyethoxymethyl (MEM), benzyl (Bn), triisopropylsilyl (TIPS), t-butyldimethylsilyl (TBDMS), t-butylmethoxyphenylsilyl (TBMPS), methanesulfonate (mesylate), and tosylate (Ts).

In certain embodiments, the substituent present on an sulfur atom is an sulfur protecting group (also referred to as a thiol protecting group). Sulfur protecting groups include, but are not limited to, —Raa, —N(Rbb)2, —C(═O)sRaa, —C(═O)Raa, —CO2Raa, —C(═O)N(Rbb)2, —C(═NRbb)Raa, —C(═NRbb)oRaa, —C(═NRbb)N(Rbb)2, —S(═O)Raa, —SO2Raa, —Si(Raa)3, —P(Rcc)2, —P(Rcc)3, —P(═O)2Raa, —P(═O)(Raa)2, —P(═O)(oRcc)2, —P(═O)2N(Rbb)2, and —P(═O)(NRbb)2, wherein Raa, Rbb, and Rcc are as defined herein. Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the substituent present on a nitrogen atom is an amino protecting group (also referred to herein as a nitrogen protecting group). Amino protecting groups include, but are not limited to, —OH, -oRaa, —N(Rcc)2, —C(═O)Raa, —C(═O)oRaa, —C(═O)N(Rcc)2, —S(═O)2Raa, —C(═NRcc)Raa, —C(═NRcc)oRaa, —C(═NRcc)N(Rcc)2, —SO2N(Rcc)2, —SO2Rcc, —SO2oRcc, —SORaa, —C(═S)N(Rcc)2, —C(═O)SRcc, —C(═S)SRcc, C1-10 alkyl, C2-10 alkenyl, C2-10 alkynyl, C3-10 carbocyclyl, 3-14-membered heterocyclyl, C6-14 aryl, and 5-14-membered heteroaryl groups, wherein each alkyl, alkenyl, alkynyl, carbocyclyl, heterocyclyl, aryl, and heteroaryl is independently substituted with 0, 1, 2, 3, 4, or 5 Rdd groups, and wherein Raa, Rbb, Rcc and Rdd are as defined herein. Amino protecting groups are well known in the art and include those described in detail in Protecting Groups in Organic Synthesis, T. W. Greene and P. G. M. Wuts, 3rd edition, John Wiley & Sons, 1999, incorporated herein by reference. Exemplary amino protecting groups include, but are not limited to amide groups (e.g., —C(═O)Raa), which include, but are not limited to, formamide and acetamide; carbamate groups (e.g., —C(═O) oRaa), which include, but are not limited to, 9-fluorenylmethyl carbamate (Fmoc), t-butyl carbamate (BOC), and benzyl carbamate (Cbz); sulfonamide groups (e.g., —S(═O)2Raa), which include, but are not limited to, p-toluenesulfonamide (Ts), methanesulfonamide (Ms), and N-[2-(trimethylsilyl) ethoxy]methylamine (SEM).

“Disease,” “disorder,” and “condition” are used interchangeably herein.

As used herein, and unless otherwise specified, the terms “treat,” “treating” and “treatment” contemplate an action that occurs while a subject is suffering from the specified disease, disorder, or condition, or symptoms thereof, which reduces the severity of the disease, disorder, or condition, or symptoms thereof, or retards or slows the progression of the disease, disorder, or condition, or symptoms thereof (“therapeutic treatment”), and also contemplates an action that occurs before a subject begins to suffer from the specified disease, disorder or condition, or symptoms thereof (“prophylactic treatment”).

In general, the “effective amount” of a compound refers to an amount sufficient to elicit the desired biological response, e.g., to treat depression, anxiety, pain, inflammation, and addiction. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder, or condition, or symptoms thereof or to delay or minimize one or more symptoms associated with the disease, disorder, or condition, or symptoms thereof. A therapeutically effective amount of a compound means an amount of therapeutic agent, alone or in combination with other therapies, which provides a therapeutic benefit in the treatment of the disease, disorder, or condition, or symptoms thereof. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder, or condition, or symptoms thereof (e.g., one or more symptoms associated with the disease, disorder, or condition), or prevent its recurrence. A prophylactically effective amount of a compound means an amount of a therapeutic agent, alone or in combination with other agents, which provides a prophylactic benefit in the prevention of the disease, disorder or condition, or symptoms thereof. The term “prophylactically effective amount” can encompass an amount that improves overall prophylaxis or enhances the prophylactic efficacy of another prophylactic agent.

The term “5-HT2A receptor”, as used herein, refers to a subclass of a family of receptors for the neurotransmitter and peripheral signal mediator serotonin. 5-HT2A receptors can mediate a plurality of central and peripheral physiologic functions of serotonin. Central nervous system effects can include mediation of hallucinogenic effects of hallucinogenic compounds.

The term “modulating 5-HT2A receptors”, as used herein, refers to the ability of a compound disclosed herein to alter the function of 5-HT2A receptors. A 5-HT2A receptor modulator may activate the activity of a 5-HT2A receptor, may activate or inhibit the activity of a 5-HT2A receptor depending on the concentration of the compound exposed to the 5-HT2A receptor, or may inhibit the activity of a 5-HT2A receptor. Such activation or inhibition may be contingent on the occurrence of a specific event, such as activation of a signal transduction pathway, and/or maybe manifest only in particular cell types. The term “modulating 5-HT2A receptors,” also refers to altering the function of a 5-HT2A receptor by increasing or decreasing the probability that a complex forms between a 5-HT2A receptor and a natural binding partner to form a multimer. A 5-HT2A receptor modulator may increase the probability that such a complex forms between the 5-HT2A receptor and the natural binding partner, may increase or decrease the probability that a complex forms between the 5-HT2A receptor and the natural binding partner depending on the concentration of the compound exposed to the 5-HT2A receptor, and or may decrease the probability that a complex forms between the 5-HT2A receptor and the natural binding partner.

The term “5-HT2A receptor-mediated disorder”, as used herein, refers to a disorder that is characterized by abnormal 5-HT2A receptor activity. A 5-HT2A receptor-mediated disorder may be completely or partially mediated by modulating 5-HT2A receptors. In particular, a 5-HT2A receptor-mediated disorder is one in which modulation of 5-HT2A receptors results in some effect on the underlying disorder e.g., administration of a 5-HT2A receptor modulator results in some improvement in at least some of the subjects being treated.

The term “glycosyl transferase” or “glycosyltransferase” as used herein, refers to any and all enzymes comprising a sequence of amino acid residues which is (i) substantially identical to the amino acid sequences constituting any glycosyl transferase polypeptide set forth herein, including, for example, SEQ ID NOs: 2, 4, 6, 8, 10, 12, 14, 16, 18, 20, 22, 24, and 26; or (ii) encoded by a nucleic acid sequence capable of hybridizing under at least moderately stringent conditions to any nucleic acid sequence encoding any glycosyl transferase set forth herein (e.g., SEQ ID NOs: SEQ ID NOs: 1, 3, 5, 7, 9, 11, 13, 15, 17, 19, 21, 23, 25, and 27), but for the use of synonymous codons.

The pGLY expression plasmids described herein are designed for expression of the GLY enzympes in Escherichia coli cells. The drawings and descriptions contain information on the functional parts including the pGLY Expression Plasmids in the form of plasmid graphic maps as wells as the coding sequence and the amino acid sequence of the GLY enzymes. The GLY enzumpes have an N-terminal His tag and their expression may require induction with IPTG.

The terms “nucleic acid sequence encoding a glycosyl transferase”, and “nucleic acid sequence encoding a glycosyl transferase polypeptide”, as may be used interchangeably herein, refer to any and all nucleic acid sequences encoding a glycosyl transferase polypeptide, including, for example, SEQ ID NOs: 1, 3, 5, 7, 9, 11, 13, 15, 17, 19, 21, 23, 25, and 27, described herein. Nucleic acid sequences encoding a glycosyl transferase polypeptide further include any and all nucleic acid sequences which (i) encode polypeptides that are substantially identical to the glycosyl transferase polypeptide sequences set forth herein (e.g., SEQ ID NOs: 2, 4, 6, 8, 10, 12, 14, 16, 18, 20, 22, 24, and 26); or (ii) hybridize to any glycosyl transferase nucleic acid sequences set forth herein (e.g., SEQ ID NOs: 1, 3, 5, 7, 9, 11, 13, 15, 17, 19, 21, 23, 25, and 27) under at least moderately stringent hybridization conditions or which would hybridize thereto under at least moderately stringent conditions but for the use of synonymous codons.

The terms “nucleic acid”, or “nucleic acid sequence”, as used herein, refer to a sequence of nucleoside or nucleotide monomers, consisting of naturally occurring bases, sugars and intersugar (backbone) linkages. The term also includes modified or substituted sequences comprising non-naturally occurring monomers or portions thereof. The nucleic acids of the present disclosure may be deoxyribonucleic nucleic acids (DNA) or ribonucleic acids (RNA) and may include naturally occurring bases including adenine, guanine, cytosine, thymidine and uracil. The nucleic acids may also contain modified bases. Examples of such modified bases include aza and deaza adenine, guanine, cytosine, thymidine and uracil, and xanthine and hypoxanthine. A sequence of nucleotide or nucleoside monomers may be referred to as a polynucleotide sequence, nucleic acid sequence, a nucleotide sequence or a nucleoside sequence.

The term “polypeptide”, as used herein in conjunction with a reference SEQ ID NO, refers to any and all polypeptides comprising a sequence of amino acid residues which is (i) substantially identical to the amino acid sequence constituting the polypeptide having such reference SEQ ID NO, or (ii) encoded by a nucleic acid sequence capable of hybridizing under at least moderately stringent conditions to any nucleic acid sequence encoding the polypeptide having such reference SEQ ID NO, but for the use of synonymous codons. A sequence of amino acid residues may be referred to as an amino acid sequence, or polypeptide sequence.

The term “nucleic acid sequence encoding a polypeptide”, as used herein in conjunction with a reference SEQ ID NO, refers to any and all nucleic acid sequences encoding a polypeptide having such reference SEQ ID NO. Nucleic acid sequences encoding a polypeptide, in conjunction with a reference SEQ ID NO, further include any and all nucleic acid sequences which (i) encode polypeptides that are substantially identical to the polypeptide having such reference SEQ ID NO; or (ii) hybridize to any nucleic acid sequences encoding polypeptides having such reference SEQ ID NO under at least moderately stringent hybridization conditions or which would hybridize thereto under at least moderately stringent conditions but for the use of synonymous codons.

By the term “substantially identical” it is meant that two sequences (e.g., amino acid sequences or nucleic acid sequences) preferably have at least 70% sequence identity (e.g., at least 75% sequence identity or at least 80% sequence identity), and more preferably have at least 85% sequence identity and most preferably have at least 95% sequence identity, for example, about 96% sequence identity, about 97% sequence identity, about 98% sequence identity, or about 99% sequence identity. In order to determine the percentage of identity between two amino acid sequences the amino acid sequences of such two sequences are aligned, using for example the alignment method of Needleman and Wunsch (J. Mol. Biol., 1970, 48:443), as revised by Smith and Waterman (Adv. Appl. Math., 1981, 2:482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12:387) BLASTP, BLASTN and FASTA (Altschul et al., J. Mol. Biol., 1990:215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89:10915-10919 using a gap opening penalty of 10 and a gap extension penalty of 0.1, so that the highest order match obtained between two sequences wherein at least 50% of the total length of one of the two sequences is involved in the alignment.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41 (% (G+C)−600/1), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5x sodium chloride/sodium citrate (SSC)/5×Denhard's solution/1.0% SDS at Tm (based on the above equation)−5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1. -6.3.6 and in: Sambrook et al., Molecular Cloning, a Laboratory Manual, Cold Spring Harbor Laboratory Press, 1989, Vol. 3.

The term “functional variant”, as used herein in reference to polynucleotides or polypeptides, refers to polynucleotides or polypeptides capable of performing the same function as a noted reference polynucleotide or polypeptide. Thus, for example, a functional variant of the polypeptides described herein (e.g., polypeptides set forth in SEQ ID NOs: 2, 4, 6, 8, 10, 12, 14, 16, 18, 20, 22, 24, and 26), refers to a polypeptide capable of performing the same function as the polypeptides described herein (e.g., polypeptides set forth in SEQ ID NOs: 2, 4, 6, 8, 10, 12, 14, 16, 18, 20, 22, 24, and 26). Functional variants include modified a polypeptide wherein, relative to a noted reference polypeptide, the modification includes a substitution, deletion or addition of one or more amino acids. In some embodiments, substitutions are those that result in a replacement of one amino acid with an amino acid having similar characteristics. Such substitutions include, without limitation (i) glutamic acid and aspartic acid; (i) alanine, serine, and threonine; (iii) isoleucine, leucine and valine, (iv) asparagine and glutamine, and (v) tryptophan, tyrosine and phenylalanine. Functional variants further include polypeptides having retained or exhibiting an enhanced psilocybin biosynthetic bioactivity.

The term “chimeric”, as used herein in the context of nucleic acids, refers to at least two linked nucleic acids which are not naturally linked. Chimeric nucleic acids include linked nucleic acids of different natural origins. For example, a nucleic acid constituting a microbial promoter linked to a nucleic acid encoding a plant polypeptide is considered chimeric. Chimeric nucleic acids also may comprise nucleic acids of the same natural origin, provided they are not naturally linked. For example a nucleic acid constituting a promoter obtained from a particular cell-type may be linked to a nucleic acid encoding a polypeptide obtained from that same cell-type, but not normally linked to the nucleic acid constituting the promoter. Chimeric nucleic acids also include nucleic acids comprising any naturally occurring nucleic acids linked to any non-naturally occurring nucleic acids.

The terms “substantially pure” and “isolated”, as may be used interchangeably herein describe a compound, e.g., a psilocybin derivative, polynucleotide or a polypeptide, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., in the case of polypeptides, by chromatography, gel electrophoresis or HPLC analysis.

The term “recovered” as used herein in association with an enzyme, protein, a chemical compound, refers to a more or less pure form of the enzyme, protein, or chemical compound.

### Methods of Use and Treatment

In an aspect, provided herein is a method of treating a disease, or symptoms thereof, in a subject in need thereof, the method comprising administering to the subject an effective amount of a compound described herein (e.g., a compound of Formula (I), Formula (Ia), Formula (Ib), Formula (Ibb), Formula (Ic), Formula (Ie), Formula (Ie-α), or Formula (Ie-β), or a subformula thereof), or a pharmaceutically acceptable salt thereof.

In some embodiments, provided herein is a method of treating a disease, or symptoms thereof, in a subject in need thereof, comprising administering to the subject an effective amount of a compound of Formula (I):

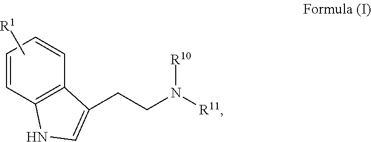

or a pharmaceutically acceptable salt thereof,
wherein
R¹ is a glycosyl group or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (I):

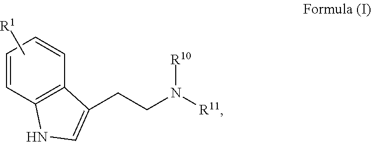

or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ia):

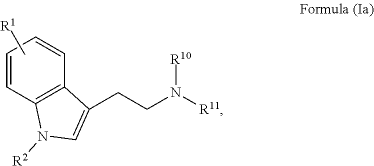

or a pharmaceutically acceptable salt thereof,
wherein
R¹ is a glycosyl group or —OH;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ib):

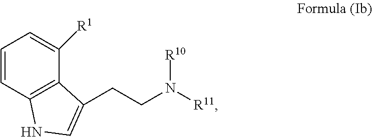

or a pharmaceutically acceptable salt thereof,
wherein
R¹ is a glycosyl group or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ib):

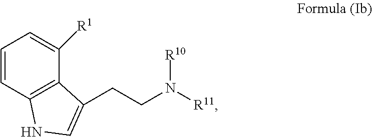

or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ibb):

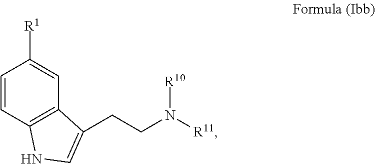

or a pharmaceutically acceptable salt thereof,
wherein
R¹ is a glycosyl group or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ibb):

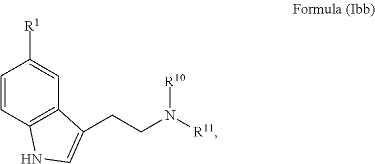

or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ic):

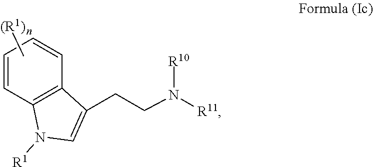

or a pharmaceutically acceptable salt thereof,
wherein
n is 1, 2, or 3;
R¹ is a glycosyl group, an —O-glycosyl group, or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl,
wherein at least one R¹ is a glycosyl group or an —O-glycosyl group.

In some embodiments, provided herein is a method of treating a disease, or symptoms thereof, in a subject in need thereof, comprising administering to the subject an effective amount of a compound of Formula (Id):

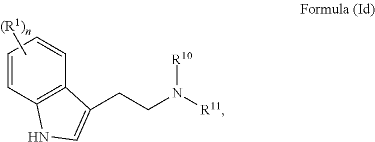

or a pharmaceutically acceptable salt thereof,
wherein
n is 1, 2, or 3;
R¹ is a glycosyl group, an —O-glycosyl group, or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl,
wherein at least one R¹ is a glycosyl group or an —O-glycosyl group.

In some embodiments, provided herein is a method of treating a disease, or symptoms thereof, in a subject in need thereof, comprising administering to the subject an effective amount of a compound of Formula (Ie):

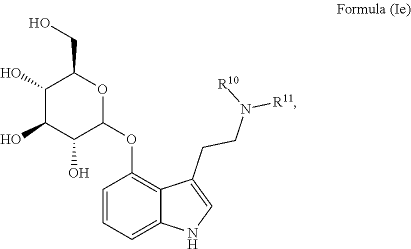

or a pharmaceutically acceptable salt thereof,
wherein
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ie-α):

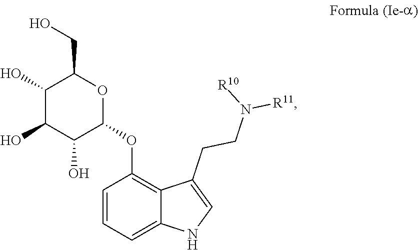

or a pharmaceutically acceptable salt thereof,
wherein
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ie-B):

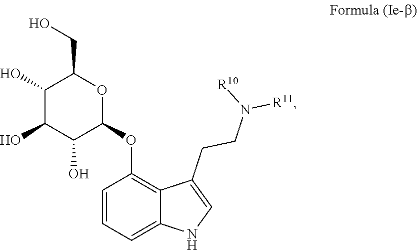

or a pharmaceutically acceptable salt thereof,
wherein
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, or 5-HO-DMT, or a pharmaceutically acceptable salt thereof.

In some embodiments, provided herein is a method of treating a disease, or symptoms thereof, in a subject in need thereof, comprising administering to the subject an effective amount of an analogue or derivative of 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, or 5-HO-DMT, or a pharmaceutically acceptable salt thereof.

In some embodiments, provided herein is a method of treating a disease, or symptoms thereof, in a subject in need thereof, comprising administering to the subject an effective amount of a glycosylated analogue or derivative of 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, or 5-HO-DMT, or a pharmaceutically acceptable salt thereof.

In some embodiments, the glycosyl group is an —O-glycosyl group.

In some embodiments, provided herein is a method of treating a disease, or symptoms thereof, in a subject in need thereof, comprising administering to the subject an effective amount of a compound of Formula (I):

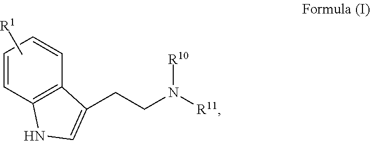

or a pharmaceutically acceptable salt thereof,
wherein
R¹ is an —O-glycosyl group or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (I):

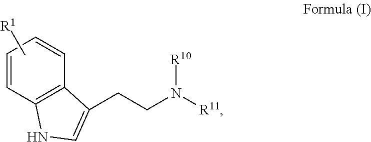

or a pharmaceutically acceptable salt thereof,
wherein
R¹ is an —O-glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ia):

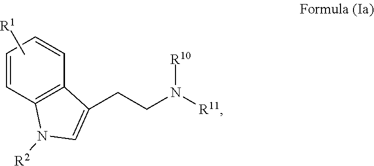

or a pharmaceutically acceptable salt thereof,
wherein
R¹ is an —O-glycosyl group or —OH;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ib):

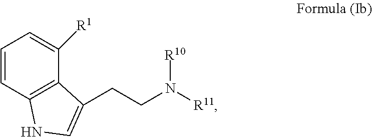

or a pharmaceutically acceptable salt thereof,
wherein
R¹ is an —O-glycosyl group or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ib):

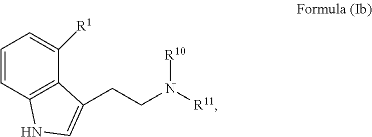

or a pharmaceutically acceptable salt thereof,
wherein
R¹ is an —O-glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ibb):

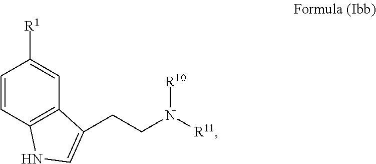

or a pharmaceutically acceptable salt thereof,
wherein
R¹ is an —O-glycosyl group or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a method of treating a disease, or symptoms thereof, in a subject in need thereof, comprising administering to the subject an effective amount of a compound of Formula (Ibb):

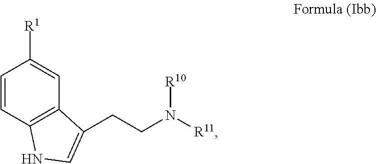

or a pharmaceutically acceptable salt thereof,
wherein
R¹ is an —O-glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, R¹⁰ is selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl.

In some embodiments, R¹⁰ is selected from unsubstituted C₁-C₆ alkyl, unsubstituted C₂-C₆ alkenyl, and unsubstituted C₃-C₁₀ cycloalkyl.

In some embodiments, R¹⁰ is unsubstituted C₁-C₆ alkyl. In some embodiments, R¹⁰ is unsubstituted C₂-C₆ alkenyl. In some embodiments, R¹⁰ is unsubstituted C₃-C₁₀ cycloalkyl.

In some embodiments, R¹⁰ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

In some embodiments, R¹⁰ is methyl, ethyl, n-propyl, isopropyl, n-butyl, tert-butyl, sec-butyl, or iso-butyl.

In some embodiments, R¹⁰ is methyl, ethyl, n-propyl, isopropyl, n-butyl, or sec-butyl.

In some embodiments, R¹⁰ is methyl. In some embodiments, R¹⁰ is ethyl. In some embodiments, R¹⁰ is n-propyl. In some embodiments, R¹⁰ is isopropyl. In some embodiments, R¹⁰ is n-butyl. In some embodiments, R¹⁰ is sec-butyl.

In some embodiments, R¹⁰ is ethenyl, 1-propenyl, 2-propenyl, 1-butenyl, 2-butenyl, or butadienyl.

In some embodiments, R¹⁰ is cyclopropyl, cyclobutyl, cyclopentyl, or cyclohexyl.

In some embodiments, R¹¹ is selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl.

In some embodiments, R¹¹ is selected from unsubstituted C₁-C₆ alkyl, unsubstituted C₂-C₆ alkenyl, and unsubstituted C₃-C₁₀ cycloalkyl.

In some embodiments, R¹¹ is unsubstituted C₁-C₆ alkyl. In some embodiments, R¹¹ is unsubstituted C₂-C₆ alkenyl. In some embodiments, R¹¹ is unsubstituted C₃-C₁₀ cycloalkyl.

In some embodiments, R¹¹ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

In some embodiments, R¹¹ is methyl, ethyl, n-propyl, isopropyl, n-butyl, tert-butyl, sec-butyl, or iso-butyl.

In some embodiments, R¹¹ is methyl, ethyl, n-propyl, isopropyl, n-butyl, or sec-butyl.

In some embodiments, R¹¹ is methyl. In some embodiments, R¹¹ is ethyl. In some embodiments, R¹¹ is n-propyl. In some embodiments, R¹¹ is isopropyl. In some embodiments, R¹¹ is n-butyl. In some embodiments, R¹¹ is sec-butyl.

In some embodiments, R¹¹ is ethenyl, 1-propenyl, 2-propenyl, 1-butenyl, 2-butenyl, or butadienyl.

In some embodiments, R¹¹ is cyclopropyl, cyclobutyl, cyclopentyl, or cyclohexyl.

In some embodiments, R¹¹ is cyclopropyl.

In some embodiments, R¹⁰ is methyl and R¹¹ is methyl.

In some embodiments, R¹⁰ is methyl and R¹¹ is ethyl.

In some embodiments, R¹⁰ is methyl and R¹¹ is n-propyl.

In some embodiments, R¹⁰ is methyl and R¹¹ is isopropyl.

In some embodiments, R¹⁰ is methyl and R¹¹ is n-butyl.

In some embodiments, R¹⁰ is methyl and R¹¹ is sec-butyl.

In some embodiments, R¹⁰ is ethyl and R¹¹ is ethyl.

In some embodiments, R¹⁰ is ethyl and R¹¹ is n-propyl.

In some embodiments, R¹⁰ is ethyl and R¹¹ is isopropyl.

In some embodiments, R¹⁰ is ethyl and R¹¹ is n-butyl.

In some embodiments, R¹⁰ is ethyl and R¹¹ is sec-butyl.

In some embodiments, R¹⁰ is n-propyl and R¹¹ is n-propyl.

In some embodiments, R¹⁰ is n-propyl and R¹¹ is isopropyl.

In some embodiments, R¹⁰ is n-propyl and R¹¹ is n-butyl.

In some embodiments, R¹⁰ is n-propyl and R¹¹ is sec-butyl.

In some embodiments, R¹⁰ is isopropyl and R¹¹ is isopropyl.

In some embodiments, R¹⁰ is isopropyl and R¹¹ is n-butyl.

In some embodiments, R¹⁰ is isopropyl and R¹¹ is sec-butyl.

In some embodiments, R¹⁰ is n-butyl and R¹¹ is n-butyl.

In some embodiments, R¹⁰ is n-butyl and R¹¹ is sec-butyl.

In some embodiments, R¹⁰ is sec-butyl and R¹¹ is sec-butyl.

In some embodiments, R¹⁰ and R¹¹ are both not methyl.

In some embodiments, R¹⁰ and R¹¹, together with the nitrogen atom to which they are attached, form a substituted 3- to 10-membered heterocyclyl or an unsubstituted 3- to 10-membered heterocyclyl.

In some embodiments, R¹⁰ and R¹¹, together with the nitrogen atom to which they are attached, form an unsubstituted 3- to 10-membered heterocyclyl.

In some embodiments, R¹⁰ and R¹¹, together with the nitrogen atom to which they are attached, form cyclopropyl, cyclobutyl, cyclopentyl, or cyclohexyl. In some embodiments, R¹⁰ and R¹¹, together with the nitrogen atom to which they are attached, form cyclopropyl. In some embodiments, R¹⁰ and R¹¹, together with the nitrogen atom to which they are attached, form cyclopentyl.

In some embodiments, the compound is

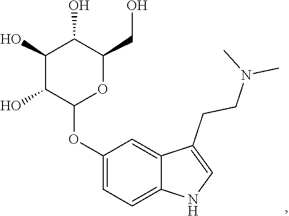 [SMILES: CN(C)CCc1c[nH]c2ccc(OC3O[C@H](CO)[C@@H](O)[C@H](O)[C@H]3O)cc12]

or a pharmaceutically acceptable salt thereof.

In some embodiments, the compound is

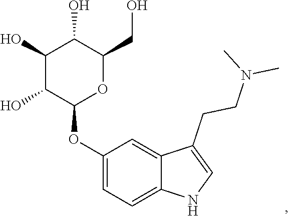 [SMILES: CN(C)CCc1c[nH]c2ccc(O[C@@H]3O[C@H](CO)[C@@H](O)[C@H](O)[C@H]3O)cc12]

or a pharmaceutically acceptable salt thereof.

In some embodiments, the compound is

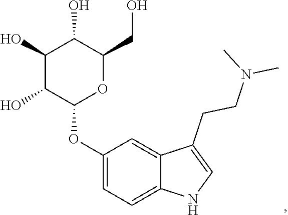 [SMILES: CN(C)CCc1c[nH]c2ccc(O[C@H]3O[C@H](CO)[C@@H](O)[C@H](O)[C@H]3O)cc12]

or a pharmaceutically acceptable salt thereof.

In some embodiments, the compound is

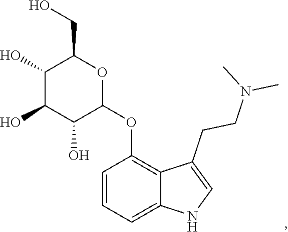 [SMILES: CN(C)CCc1c[nH]c2cccc(OC3O[C@H](CO)[C@@H](O)[C@H](O)[C@H]3O)c12]

or a pharmaceutically acceptable salt thereof.

In some embodiments, the compound is

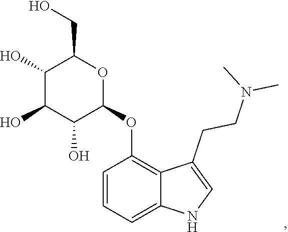 [SMILES: CN(C)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

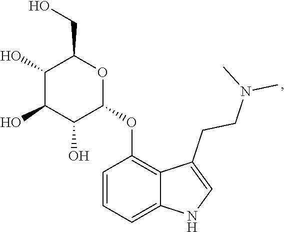 [SMILES: CN(C)CCc1c[nH]c2cccc(O[C@H]3O[C@H](CO)[C@@H](O)[C@H](O)[C@H]3O)c12]

or a pharmaceutically acceptable salt thereof.

In some embodiments, the compound is

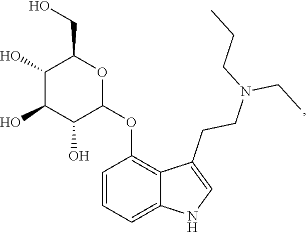 [SMILES: CCCN(CC)CCc1c[nH]c2cccc(OC3O[C@H](CO)[C@@H](O)[C@H](O)[C@H]3O)c12]

or a pharmaceutically acceptable salt thereof.

In some embodiments, the compound is

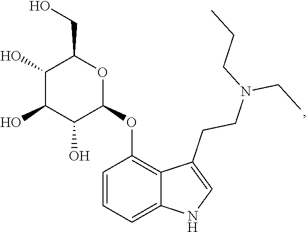 [SMILES: CCCN(CC)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

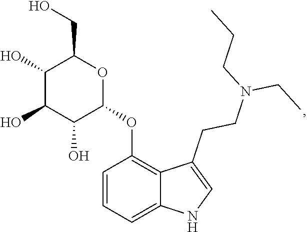 [SMILES: CCCN(CC)CCc1c[nH]c2cccc(O[C@H]3O[C@H](CO)[C@@H](O)[C@H](O)[C@H]3O)c12]

or a pharmaceutically acceptable salt thereof.

In some embodiments, the compound is

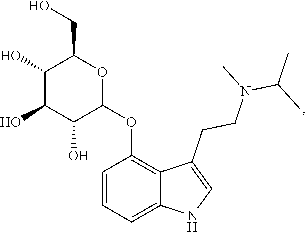 [SMILES: CC(C)N(C)CCc1c[nH]c2cccc(OC3O[C@H](CO)[C@@H](O)[C@H](O)[C@H]3O)c12]

or a pharmaceutically acceptable salt thereof.

In some embodiments, the compound is

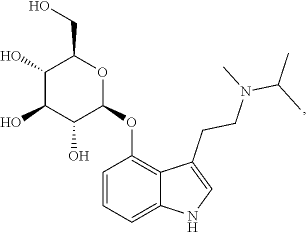 [SMILES: CC(C)N(C)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

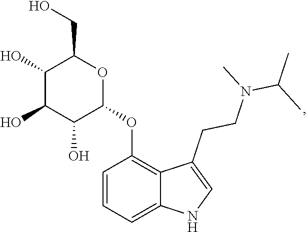 [SMILES: CC(C)N(C)CCc1c[nH]c2cccc(O[C@H]3O[C@H](CO)[C@@H](O)[C@H](O)[C@H]3O)c12]

or a pharmaceutically acceptable salt thereof.

In some embodiments, the compound is

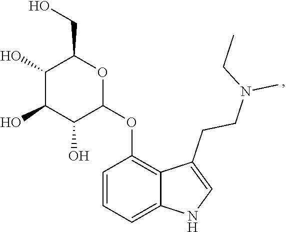 [SMILES: CCN(C)CCc1c[nH]c2cccc(OC3O[C@H](CO)[C@@H](O)[C@H](O)[C@H]3O)c12]

or a pharmaceutically acceptable salt thereof.

In some embodiments, the compound is

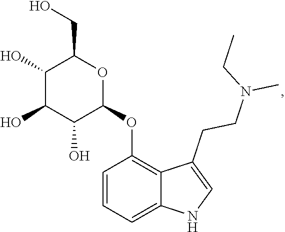 [SMILES: CCN(C)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

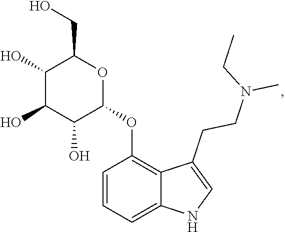 [SMILES: CCN(C)CCc1c[nH]c2cccc(O[C@H]3O[C@H](CO)[C@@H](O)[C@H](O)[C@H]3O)c12]

or a pharmaceutically acceptable salt thereof.

In some embodiments, the disease is depression, anxiety, pain, inflammation, addiction, an autoimmune disease, an eating disorder, or an obsessive compulsive disorder. In some embodiments, the disease is depression, anxiety, pain, inflammation, or addiction. In some embodiments, the disease is an autoimmune disease, an eating disorder, or an obsessive compulsive disorder

In some embodiments, the disease is autism, anxiety, depression, bipolar disorder, attention deficit disorder, attention deficit hyperactivity disorder (ADHD), schizophrenia, a psychotic disorder, a psychotic symptom, social withdrawal, obsessive-compulsive disorder (OCD), phobia, post-traumatic stress syndrome, a behavior disorder, an impulse control disorder, a substance abuse disorder (e.g., a withdrawal symptom, opiate addiction, nicotine addiction, and ethanol addition), a sleep disorder, a memory disorder (e.g., a deficit, loss, or reduced ability to make new memories), a learning disorder, Tourette's syndrome, epilepsy, cerebral ischemia, special learning disorders, seizures, post-stroke convulsions, brain ischemia, migraine, stroke, spinal cord injury, peripheral neuropathy, acute neuropathic pain, and chronic neuropathic pain.

Also provided are methods for treating social withdrawal in conditions including, but not limited to, social anxiety disorder, avoidant personality disorder, and schizotypal personality disorder.

In some embodiments, methods are provided for treating neuropathic pain. The neuropathic pain may be acute or chronic. In some cases, the neuropathic pain may be associated with a condition such as herpes, HIV, traumatic nerve injury, stroke, post-ischemia, fibromyalgia, reflex sympathetic dystrophy, complex regional pain syndrome, spinal cord injury, sciatica, phantom limb pain, diabetic neuropathy, and cancer chemotherapeutic-induced neuropathic pain. Methods for enhancing pain relief and for providing analgesia to a patient are also contemplated.

Neuropathic pain or neuropathy is caused by injury to nerves (e.g., autonomic nerves, motor nervies, or sensory nerves). In some embodiments, neuropathic pain is caused by metabolic nerve damage. Metabolic neuropathies include, but are not limited to, diabetic neuropathy, drug-induced neuropathy, and post herpetic neuropathy. In some embodiments, neuropathic pain is caused by a discrete nerve injury. Discrete nerve injuries include, but are not limited to, post-amputation pain, post-surgical pain, and nerve entrapment injuries (e.g., neuropathic back pain).

In some embodiments, the subject has cancer. In some embodiments, the subject has been treated with an anti-cancer therapy. In some embodiments, the disease is anti-cancer therapy-induced neuropathic pain. In some embodiments, the anti-cancer therapy is selected from: administration of an immunotherapeutic agent, a chemotherapeutic agent, a growth inhibitory agent, a cytotoxic agent, or a combination thereof; radiation therapy; surgery; and combinations thereof. In some embodiments, the disease is chemotherapeutic-induced pain. In some embodiments, the disease is cancer chemotherapy-induced pain.

In some embodiments, the pain is treatment resistant pain. In some embodiments, the pain is refractory pain. In some embodiments, the pain is treatment resistant refractory pain. In some embodiments, the pain is opioid resistant pain. In some embodiments, the pain is opioid resistant refractory pain. In some embodiments, the opioid is codeine, fentanyl, hydromorphone, methadone, morphine, or oxycodone, or a pharmaceutically acceptable salt thereof. In some embodiments, the opioid is hydromorphone, morphine, or oxycodone, or a pharmaceutically acceptable salt thereof.

In a further embodiment, a method of treating depression comprising administering an a compound is provided. In some embodiments, the treatment may relieve depression or a symptom of depression without affecting behavior or motor coordination and without inducing or promoting seizure activity. Exemplary depression conditions that are expected to be treated according to this aspect of the invention include, but are not limited to, major depressive disorder, dysthymic disorder, psychotic depression, postpartum depression, premenstrual syndrome, premenstrual dysphoric disorder, seasonal affective disorder (SAD), bipolar disorder (or manic depressive disorder), mood disorder, and depressions caused by chronic medical conditions such as cancer or chronic pain, chemotherapy, chronic stress, and post traumatic stress disorders. In addition, patients suffering from any form of depression often experience anxiety. Various symptoms associated with anxiety include fear, panic, heart palpitations, shortness of breath, fatigue, nausea, and headaches among others. Anxiety or any of the symptoms thereof may be treated by administering a compound as described herein.

Compounds of the invention may be useful to control food intake in a patient and thus used to treat eating disorders in such patients. Eating disorders can include but not be limited to overeating resulting in obesity, bulimia, compulsive eating, pica, insufficient eating leading to anorexia, improper eating resultant of diabetes, and abnormalities in the perception of taste (ageusia/dysgeusia) that lead to abnormal eating patterns. It is also understood to one of skill in the art that eating disorders may also involve disorders of taste and smell and thus the invention also encompasses methods of treating taste and smell disorders.

Compounds of the disclosure may be used to treat binge eating disorder. Binge eating disorder is a form of Eating Disorder Not Otherwise Specified. As defined by the DSM-IV-TR, it is characterized by recurrent binge eating episodes. Such episodes include eating larger amounts of food than normal during a short period of time (for instance, within a two hour period) and a lack of control over eating during the binge episode (for instance, one cannot stop eating). According to the DSM-IV-TR, binge eating disorders are associated with three or more of the following symptoms: eating until uncomfortably full; eating large amounts of food when not physically hungry; eating much more rapidly than normal; eating alone on account of embarrassment over how much one is eating; and feeling disgusted, depressed or guilty after overeating. Additionally, individuals with binge eating disorder feel distress about their binging behavior. The DSM-IV-TR also characterizes binge eating to occur, on average, at least 2 days a week for six months, while not being associated with the regular use of inappropriate compensatory behaviors such as purging or excessive exercise and not occurring exclusively during the course of bulimia nervosa or anorexia nervosa. As used herein “depression” includes major depressive disorder, dysthymic disorder, depressive disorder not otherwise specified (for instance, premenstrual dysphoric disorder), and depressive episodes that may be present in another disorder (e.g. as in other mood disorders such as bipolar disorder or a mood disorder due to a general medical condition).

In various embodiments of the invention, the obsessive-compulsive spectrum disorder (OCSD) comprises body dysmorphic disorder (BDD), compulsive skin picking, Tourette syndrome, Attention Deficit/Hyperactivity Disorder, anorexia nervosa, antisocial personality disorder (ASPD), autism, basal ganglia disorder, borderline personality disorder (BPD), bulimia, depersonalization disorder, epilepsy, Huntington's disease, hypochondriasis, kelptomania, personality disorder, pathologic gambling, sexual compulsions, Sydenham's chorea, torticollis, trichotillomania, or Pediatric Autoimmune Neuropsychiatric Disorders Associated with Streptococcal infections.

Compounds of the disclosure may be used to treat an autoimmune disease, wherein the autoimmune disease comprises Ankylosing Spondylitis, Chagas disease, chronic obstructive pulmonary disease, Crohns Disease, Dermatomyositis, Endometriosis, Goodpasture's syndrome, Graves' disease, Guillain-Barré syndrome, Hashimoto's disease, Hidradenitis suppurativa, Kawasaki disease, IgA nephropathy, idiopathic thrombocytopenia purpura, interstitial cystitis, lupus erythematosus, mixed connective tissue disease, morphea, myasthenia gravis, narcolepsy, neuromyotonia, pemphigus vulgaris, pernicious anaemia, psoriasis, psoriatic Arthritis, polymyositis, primary biliary cirrhosis, relapsing polychondritis, rheumatoid arthritis, schizophrenia, scleroderma, Sjögren's syndrome, stiff person syndrome, temporal arteritis, ulcerative colitis, vasculitis, vitiligo, multiple sclerosis, rheumatoid arthritis, systemic lupus erythematosus, psoriasis, ankylosing spondilitis, scleroderma, Type I diabetes, psoriatic arthritis, osteoarthritis, inflammatory bowel disease, atopic dermatitis and asthma, or Wegener's granulomatosis.

In an aspect, the compounds of the present disclosure may be used to be contacted with a 5-HT2A receptor to thereby modulate the 5-HT2A receptor. Such contacting includes bringing a compound of the present disclosure and 5-HT2A receptor together under in vitro conditions, for example, by introducing the compounds in a sample containing a 5-HT2A receptor, for example, a sample containing purified 5-HT2A receptors, or a sample containing cells comprising 5-HT2A receptors. In vitro conditions further include the conditions described in Example 3 hereof. Contacting further includes bringing a compound of the present disclosure and 5-HT2A receptor together under in vivo conditions. Such in vivo conditions include the administration to an animal or human subject, for example, of a pharmaceutically effective amount of the compound of the present disclosure, when the compound is formulated together with a pharmaceutically active carrier, diluent or excipient, as hereinbefore described, to thereby treat the subject. Upon having contacted the 5-HT2A receptor, the compound may activate the 5-HT2A receptor or inhibit the 5-HT2A receptor.

Thus, in a further aspect, the condition that may be treated in accordance herewith can be any 5-HT2A receptor mediated disorder.

In some embodiments, the effective amount of the compound (e.g., the compound of Formula (I), Formula (Ia), Formula (Ib), Formula (Ibb), Formula (Ic), Formula (Ie), Formula (Ie-α), or Formula (Ie-β), or a subformula thereof), is about 0.1 mg/kg to about 10 mg/kg, about 0.2 mg/kg to about 10 mg/kg, about 0.3 mg/kg to about 10 mg/kg, about 0.4 mg/kg to about 10 mg/kg, about 0.5 mg/kg to about 10 mg/kg, about 0.6 mg/kg to about 10 mg/kg, about 0.7 mg/kg to about 10 mg/kg, about 0.8 mg/kg to about 10 mg/kg, about 0.9 mg/kg to about 10 mg/kg, about 0.9 mg/kg to about 10 mg/kg, about 1 mg/kg to 10 mg/kg, about 0.1 mg/kg to about 1 mg/kg, about 0.1 mg/kg to about 0.75 mg/kg, about 0.1 mg/kg to about 0.2 mg/kg, about 0.1 mg/kg to about 0.25 mg/kg, about 0.5 mg/kg to about 1 mg/kg, about 0.75 mg/kg to about 2 mg/kg, about 1 mg/kg to about 2 mg/kg, about 2 mg/kg to about 5 mg/kg, about 5 mg/kg to about 7.5 mg/kg, about 7.5 mg/kg to about 10 mg/kg, about 0.1 mg/kg to about 20 mg/kg, about 0.2 mg/kg to about 20 mg/kg, about 0.3 mg/kg to about 20 mg/kg, about 0.4 mg/kg to about 20 mg/kg, about 0.5 mg/kg to about 20 mg/kg, about 0.6 mg/kg to about 20 mg/kg, about 0.7 mg/kg to about 20 mg/kg, about 0.8 mg/kg to about 20 mg/kg, about 0.9 mg/kg to about 20 mg/kg, about 0.9 mg/kg to about 20 mg/kg, about 1 mg/kg to about 20 mg/kg, about 5 mg/kg to about 20 mg/kg, about 10 mg/kg to about 20 mg/kg, about 15 mg/kg to about 20 mg/kg, or about 7.5 mg/kg to about 10 mg/kg.

In some embodiments, the compound is administered by inhalation, oral (enteral) administration, parenteral (by injection) administration, rectal administration, transdermal administration, intradermal administration, intrathecal administration, subcutaneous (SC) administration, intravenous (IV) administration, intramuscular (IM) administration, or intranasal administration. In some embodiments, the compound is administered orally, subcutaneously, intravenously, or intramuscularly. In some embodiments, the compound is administered orally. In some embodiments, the compound is administered intravenously. In some embodiments, the compound is administered chronically. In some embodiments, the compound is administered continuously. In some embodiments, the compound is administered by continuous intravenous infusion.

### Compounds

In some embodiments, provided herein is a compound of Formula (I):

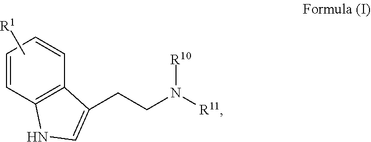

or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a compound of Formula (Ia):

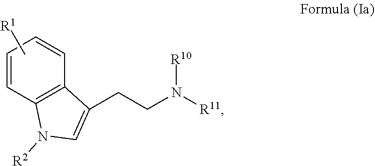

or a pharmaceutically acceptable salt thereof,
wherein
R¹ is a glycosyl group or —OH;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a compound of Formula (Ib):

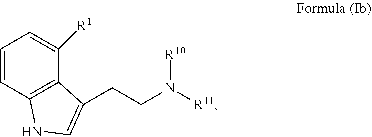

or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a compound of Formula (Ibb):

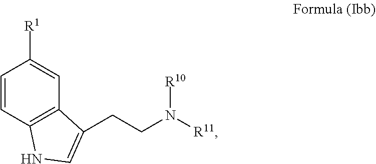

or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a compound of Formula (Ic):

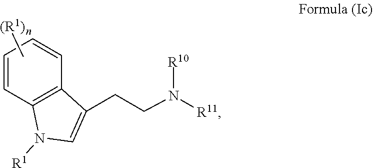

or a pharmaceutically acceptable salt thereof,
wherein
n is 1, 2, or 3;
R¹ is a glycosyl group, an —O-glycosyl group, or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl,
wherein at least one R¹ is a glycosyl group or an —O-glycosyl group.

In some embodiments, provided herein is a compound of Formula (Id):

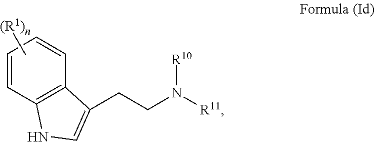

or a pharmaceutically acceptable salt thereof,
wherein
n is 1, 2, or 3;
R¹ is a glycosyl group, an —O-glycosyl group, or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl,
wherein at least one R¹ is a glycosyl group or an —O-glycosyl group

In some embodiments, provided herein is a compound of Formula (Ie):

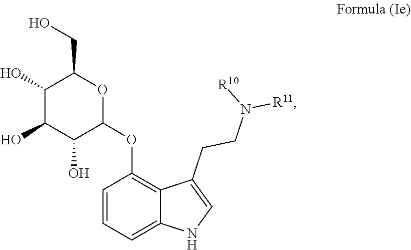

or a pharmaceutically acceptable salt thereof,
wherein
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl

In some embodiments, provided herein is a compound of Formula (Ie-α):

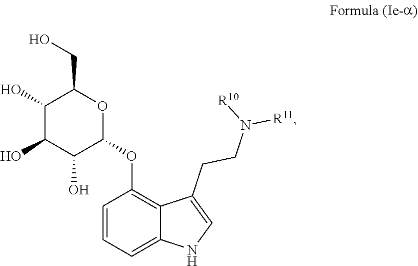

or a pharmaceutically acceptable salt thereof,
wherein
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a compound of Formula (Ie-β):

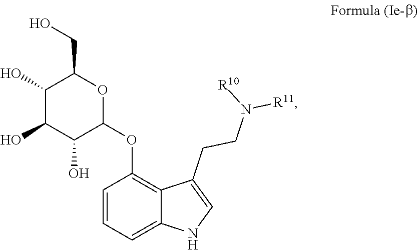

or a pharmaceutically acceptable salt thereof,
wherein
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a glycosylated analogue or derivative of 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, or 5-HO-DMT, or a pharmaceutically acceptable salt thereof.

In some embodiments, the glycosyl group is an —O-glycosyl group.

In some embodiments, R¹⁰ is selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl.

In some embodiments, R¹⁰ is selected from unsubstituted C₁-C₆ alkyl, unsubstituted C₂-C₆ alkenyl, and unsubstituted C₃-C₁₀ cycloalkyl.

In some embodiments, R¹⁰ is unsubstituted C₁-C₆ alkyl. In some embodiments, R¹⁰ is unsubstituted C₂-C₆ alkenyl. In some embodiments, R¹⁰ is unsubstituted C₃-C₁₀ cycloalkyl.

In some embodiments, R¹⁰ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

In some embodiments, R¹⁰ is methyl, ethyl, n-propyl, isopropyl, n-butyl, tert-butyl, sec-butyl, or iso-butyl.

In some embodiments, R¹⁰ is methyl, ethyl, n-propyl, isopropyl, n-butyl, or sec-butyl.

In some embodiments, R¹⁰ is methyl. In some embodiments, R¹⁰ is ethyl. In some embodiments, R¹⁰ is n-propyl. In some embodiments, R¹⁰ is isopropyl. In some embodiments, R¹⁰ is n-butyl. In some embodiments, R¹⁰ is sec-butyl.

In some embodiments, R¹⁰ is ethenyl, 1-propenyl, 2-propenyl, 1-butenyl, 2-butenyl, or butadienyl.

In some embodiments, R¹⁰ is cyclopropyl, cyclobutyl, cyclopentyl, or cyclohexyl.

In some embodiments, R¹¹ is selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl.

In some embodiments, R¹¹ is selected from unsubstituted C₁-C₆ alkyl, unsubstituted C₂-C₆ alkenyl, and unsubstituted C₃-C₁₀ cycloalkyl.

In some embodiments, R¹¹ is unsubstituted C₁-C₆ alkyl. In some embodiments, R¹¹ is unsubstituted C₂-C₆ alkenyl. In some embodiments, R¹¹ is unsubstituted C₃-C₁₀ cycloalkyl.

In some embodiments, R¹¹ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

In some embodiments, R¹¹ is methyl, ethyl, n-propyl, isopropyl, n-butyl, tert-butyl, sec-butyl, or iso-butyl.

In some embodiments, R¹¹ is methyl, ethyl, n-propyl, isopropyl, n-butyl, or sec-butyl.

In some embodiments, R¹¹ is methyl. In some embodiments, R¹¹ is ethyl. In some embodiments, R¹¹ is n-propyl. In some embodiments, R¹¹ is isopropyl. In some embodiments, R¹¹ is n-butyl. In some embodiments, R¹¹ is sec-butyl.

In some embodiments, R¹¹ is ethenyl, 1-propenyl, 2-propenyl, 1-butenyl, 2-butenyl, or butadienyl.

In some embodiments, R¹¹ is cyclopropyl, cyclobutyl, cyclopentyl, or cyclohexyl.

In some embodiments, R¹⁰ is methyl and R¹¹ is methyl.

In some embodiments, R¹⁰ is methyl and R¹¹ is ethyl.

In some embodiments, R¹⁰ is methyl and R¹¹ is n-propyl.

In some embodiments, R¹⁰ is methyl and R¹¹ is isopropyl.

In some embodiments, R¹⁰ is methyl and R¹¹ is n-butyl.

In some embodiments, R¹⁰ is methyl and R¹¹ is sec-butyl.

In some embodiments, R¹⁰ is ethyl and R¹¹ is ethyl.

In some embodiments, R¹⁰ is ethyl and R¹¹ is n-propyl.

In some embodiments, R¹⁰ is ethyl and R¹¹ is isopropyl.

In some embodiments, R¹⁰ is ethyl and R¹¹ is n-butyl.

In some embodiments, R¹⁰ is ethyl and R¹¹ is sec-butyl.

In some embodiments, R¹⁰ is n-propyl and R¹¹ is n-propyl.

In some embodiments, R¹⁰ is n-propyl and R¹¹ is isopropyl.

In some embodiments, R¹⁰ is n-propyl and R¹¹ is n-butyl.

In some embodiments, R¹⁰ is n-propyl and R¹¹ is sec-butyl.

In some embodiments, R¹⁰ is isopropyl and R¹¹ is isopropyl.

In some embodiments, R¹⁰ is isopropyl and R¹¹ is n-butyl.

In some embodiments, R¹⁰ is isopropyl and R¹¹ is sec-butyl.

In some embodiments, R¹⁰ is n-butyl and R¹¹ is n-butyl.

In some embodiments, R¹⁰ is n-butyl and R¹¹ is sec-butyl.

In some embodiments, R¹⁰ is sec-butyl and R¹¹ is sec-butyl.

In some embodiments, R¹⁰ and R¹¹ are both not methyl.

In some embodiments, R¹⁰ and R¹¹, together with the nitrogen atom to which they are attached, form a substituted 3- to 10-membered heterocyclyl or an unsubstituted 3- to 10-membered heterocyclyl.

In some embodiments, R¹⁰ and R¹¹, together with the nitrogen atom to which they are attached, form an unsubstituted 3- to 10-membered heterocyclyl.

In some embodiments, R¹⁰ and R¹¹, together with the nitrogen atom to which they are attached, form cyclopropyl, cyclobutyl, cyclopentyl, or cyclohexyl.

In some embodiments, provided herein is a compound of Formula (Ia)

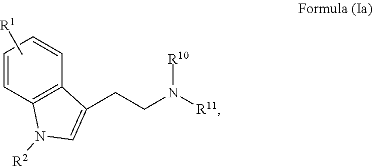

or a pharmaceutically acceptable salt thereof,
   wherein
R¹ is selected from the group consisting of a glycosyl group, —O-glycosyl group and —OH;
R² is glycosyl group; and
R¹⁰ and R¹¹ are each independently substituted or unsubstituted C₁-C₆ alkyl.

In some embodiments, R¹⁰ is methyl and R¹¹ is methyl.

In some embodiments, R¹⁰ is methyl and R¹¹ is ethyl.

In some embodiments, R¹⁰ is methyl and R¹¹ is n-propyl.

In some embodiments, R¹⁰ is methyl and R¹¹ is isopropyl.

In some embodiments, R¹⁰ is methyl and R¹¹ is n-butyl.

In some embodiments, R¹⁰ is methyl and R¹¹ is sec-butyl.

In some embodiments, R¹⁰ is ethyl and R¹¹ is ethyl.

In some embodiments, R¹⁰ is ethyl and R¹¹ is n-propyl.

In some embodiments, R¹⁰ is ethyl and R¹¹ is isopropyl.

In some embodiments, R¹⁰ is ethyl and R¹¹ is n-butyl.

In some embodiments, R¹⁰ is ethyl and R¹¹ is sec-butyl.

In some embodiments, R¹⁰ is n-propyl and R¹¹ is n-propyl.

In some embodiments, R¹⁰ is n-propyl and R¹¹ is isopropyl.

In some embodiments, R¹⁰ is n-propyl and R¹¹ is n-butyl.

In some embodiments, R¹⁰ is n-propyl and R¹¹ is sec-butyl.

In some embodiments, R¹⁰ is isopropyl and R¹¹ is isopropyl.

In some embodiments, R¹⁰ is isopropyl and R¹¹ is n-butyl.

In some embodiments, R¹⁰ is isopropyl and R¹¹ is sec-butyl.

In some embodiments, R¹⁰ is n-butyl and R¹¹ is n-butyl.

In some embodiments, R¹⁰ is n-butyl and R¹¹ is sec-butyl.

In some embodiments, R¹⁰ is sec-butyl and R¹¹ is sec-butyl.

In some embodiments, R¹⁰ is methyl.

In some embodiments, R¹⁰ is ethyl.

In some embodiments, R¹⁰ is propyl.

In some embodiments, R¹⁰ and R¹¹ are both not methyl.

In some embodiments, R¹⁰ is butyl.

In some embodiments, provided herein is a compound of Formula (Ib)

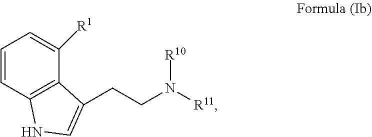

or a pharmaceutically acceptable salt thereof
   wherein
R¹ is —O-glycosyl group; and
R¹⁰ and R¹¹ are each independently substituted or unsubstituted C₁-C₆ alkyl.

In some embodiments, provided herein is a compound of Formula (Ibb)

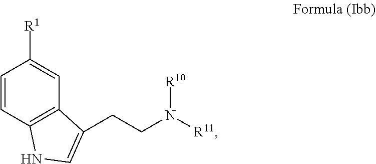

or a pharmaceutically acceptable salt thereof
   wherein
R¹ is —O-glycosyl group; and
R¹⁰ and R¹¹ are each independently substituted or unsubstituted C₁-C₆ alkyl.

In some embodiments, R¹⁰ is methyl and R¹¹ is methyl.

In some embodiments, R¹⁰ is methyl and R¹¹ is ethyl.

In some embodiments, R¹⁰ is methyl and R¹¹ is n-propyl.

In some embodiments, R¹⁰ is methyl and R¹¹ is isopropyl.

In some embodiments, R¹⁰ is methyl and R¹¹ is n-butyl.

In some embodiments, R¹⁰ is methyl and R¹¹ is sec-butyl.

In some embodiments, R¹⁰ is ethyl and R¹¹ is ethyl.

In some embodiments, R¹⁰ is ethyl and R¹¹ is n-propyl.

In some embodiments, R¹⁰ is ethyl and R¹¹ is isopropyl.

In some embodiments, R¹⁰ is ethyl and R¹¹ is n-butyl.

In some embodiments, R¹⁰ is ethyl and R¹¹ is sec-butyl.

In some embodiments, R¹⁰ is n-propyl and R¹¹ is n-propyl.

In some embodiments, R¹⁰ is n-propyl and R¹¹ is isopropyl.

In some embodiments, R¹⁰ is n-propyl and R¹¹ is n-butyl.

In some embodiments, R¹⁰ is n-propyl and R¹¹ is sec-butyl.

In some embodiments, R¹⁰ is isopropyl and R¹¹ is isopropyl.

In some embodiments, R¹⁰ is isopropyl and R¹¹ is n-butyl.

In some embodiments, R¹⁰ is isopropyl and R¹¹ is sec-butyl.

In some embodiments, R¹⁰ is n-butyl and R¹¹ is n-butyl.

In some embodiments, R¹⁰ is n-butyl and R¹¹ is sec-butyl.

In some embodiments, R¹⁰ is sec-butyl and R¹¹ is sec-butyl.

In some embodiments, R¹⁰ and R¹¹ are both not methyl.

In some embodiments, R¹⁰ is methyl.

In some embodiments, R¹⁰ is ethyl.

In some embodiments, R¹⁰ is propyl.

In some embodiments, R¹⁰ and R¹¹ are both not methyl.

In some embodiments, R¹⁰ is butyl.

In some embodiments, provided herein is a compound of Formula (Ib), or a pharmaceutically acceptable salt thereof.

In some embodiments, provided herein is a compound of Formula (Ibb), or a pharmaceutically acceptable salt thereof.

In some embodiments, provided herein is a compound of Formula (Ic)

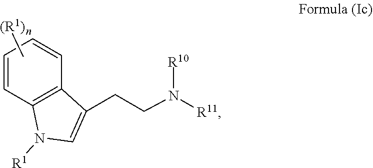

or a pharmaceutically acceptable salt thereof,
wherein
each R¹ is independently selected from the group consisting of a glycosyl group, —O-glycosyl group and —OH;
each n is independently 1-3; and
R¹⁰ and R¹¹ are each independently substituted or unsubstituted C₁-C₆ alkyl.

In some embodiments, R¹⁰ is methyl and R¹¹ is methyl.

In some embodiments, R¹⁰ is methyl and R¹¹ is ethyl.

In some embodiments, R¹⁰ is methyl and R¹¹ is n-propyl.

In some embodiments, R¹⁰ is methyl and R¹¹ is isopropyl.

In some embodiments, R¹⁰ is methyl and R¹¹ is n-butyl.

In some embodiments, R¹⁰ is methyl and R¹¹ is sec-butyl.

In some embodiments, R¹⁰ is ethyl and R¹¹ is ethyl.

In some embodiments, R¹⁰ is ethyl and R¹¹ is n-propyl.

In some embodiments, R¹⁰ is ethyl and R¹¹ is isopropyl.

In some embodiments, R¹⁰ is ethyl and R¹¹ is n-butyl.

In some embodiments, R¹⁰ is ethyl and R¹¹ is sec-butyl.

In some embodiments, R¹⁰ is n-propyl and R¹¹ is n-propyl.

In some embodiments, R¹⁰ is n-propyl and R¹¹ is isopropyl.

In some embodiments, R¹⁰ is n-propyl and R¹¹ is n-butyl.

In some embodiments, R¹⁰ is n-propyl and R¹¹ is sec-butyl.

In some embodiments, R¹⁰ is isopropyl and R¹¹ is isopropyl.

In some embodiments, R¹⁰ is isopropyl and R¹¹ is n-butyl.

In some embodiments, R¹⁰ is isopropyl and R¹¹ is sec-butyl.

In some embodiments, R¹⁰ is n-butyl and R¹¹ is n-butyl.

In some embodiments, R¹⁰ is n-butyl and R¹¹ is sec-butyl.

In some embodiments, R¹⁰ is sec-butyl and R¹¹ is sec-butyl.

In some embodiments, R¹⁰ is methyl.

In some embodiments, R¹⁰ is ethyl.

In some embodiments, R¹⁰ is propyl.

In some embodiments, R¹ or R² is a lower oligo saccharide. In some embodiments, R¹ or R² is a monosaccharide or a disaccharide.

In some embodiments, R¹⁰ and R¹¹ are both not methyl.

In some embodiments, R¹⁰ is butyl.

In some embodiments, provided herein is a compound of Formula (Id)

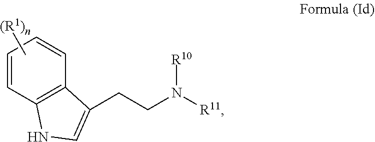

or a pharmaceutically acceptable salt thereof
wherein
each R¹ is independently selected from the group consisting of a glycosyl group, —O-glycosyl group and —OH, and at least one R¹ is a glycosyl group or a —O-glycosyl group;
n is 1-3; and
R¹⁰ and R¹¹ are each independently substituted or unsubstituted C₁-C₆ alkyl.

In some embodiments, R¹⁰ is methyl and R¹¹ is methyl.

In some embodiments, R¹⁰ is methyl and R¹¹ is ethyl.

In some embodiments, R¹⁰ is methyl and R¹¹ is n-propyl.

In some embodiments, R¹⁰ is methyl and R¹¹ is isopropyl.

In some embodiments, R¹⁰ is methyl and R¹¹ is n-butyl.

In some embodiments, R¹⁰ is methyl and R¹¹ is sec-butyl.

In some embodiments, R¹⁰ is ethyl and R¹¹ is ethyl.

In some embodiments, R¹⁰ is ethyl and R¹¹ is n-propyl.

In some embodiments, R¹⁰ is ethyl and R¹¹ is isopropyl.

In some embodiments, R¹⁰ is ethyl and R¹¹ is n-butyl.

In some embodiments, R¹⁰ is ethyl and R¹¹ is sec-butyl.

In some embodiments, R¹⁰ is n-propyl and R¹¹ is n-propyl.

In some embodiments, R¹⁰ is n-propyl and R¹¹ is isopropyl.

In some embodiments, R¹⁰ is n-propyl and R¹¹ is n-butyl.

In some embodiments, R¹⁰ is n-propyl and R¹¹ is sec-butyl.

In some embodiments, R¹⁰ is isopropyl and R¹¹ is isopropyl.

In some embodiments, R¹⁰ is isopropyl and R¹¹ is n-butyl.

In some embodiments, R¹⁰ is isopropyl and R¹¹ is sec-butyl.

In some embodiments, R¹⁰ is n-butyl and R¹¹ is n-butyl.

In some embodiments, R¹⁰ is n-butyl and R¹¹ is sec-butyl.

In some embodiments, R¹⁰ is sec-butyl and R¹¹ is sec-butyl.

In some embodiments, R¹⁰ is methyl.

In some embodiments, R¹⁰ is ethyl.

In some embodiments, R¹⁰ is propyl.

In some embodiments, R¹⁰ and R¹¹ are both not methyl.

In some embodiments, R¹⁰ is butyl.

In some embodiments, provided herein is a compound of Formula (Ie)

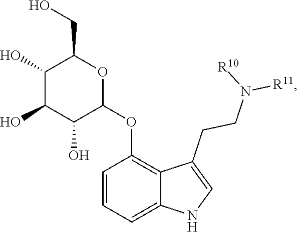

or a pharmaceutically acceptable salt thereof,
wherein
R¹ is glycosyl group; and
R¹⁰ and R¹¹ are each independently substituted or unsubstituted C₁-C₆ alkyl.

In some embodiments, provided herein is a compound of Formula (Ie-α)

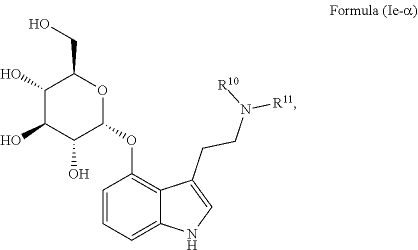

or a pharmaceutically acceptable salt thereof,
wherein
R¹ is glycosyl group; and
R¹⁰ and R¹¹ are each independently substituted or unsubstituted C₁-C₆ alkyl.

In some embodiments, provided herein is a compound of Formula (Ie-β)

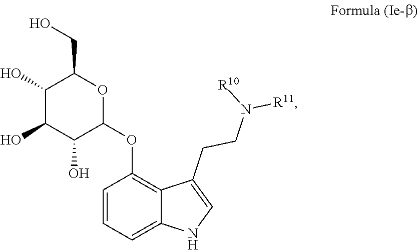

or a pharmaceutically acceptable salt thereof,
wherein
R¹ is glycosyl group; and
R¹⁰ and R¹¹ are each independently substituted or unsubstituted C₁-C₆ alkyl.

In some embodiments, R¹⁰ is methyl and R¹¹ is methyl.

In some embodiments, R¹⁰ is methyl and R¹¹ is ethyl.

In some embodiments, R¹⁰ is methyl and R¹¹ is n-propyl.

In some embodiments, R¹⁰ is methyl and R¹¹ is isopropyl.

In some embodiments, R¹⁰ is methyl and R¹¹ is n-butyl.

In some embodiments, R¹⁰ is methyl and R¹¹ is sec-butyl.

In some embodiments, R¹⁰ is ethyl and R¹¹ is ethyl.

In some embodiments, R¹⁰ is ethyl and R¹¹ is n-propyl.

In some embodiments, R¹⁰ is ethyl and R¹¹ is isopropyl.

In some embodiments, R¹⁰ is ethyl and R¹¹ is n-butyl.

In some embodiments, R¹⁰ is ethyl and R¹¹ is sec-butyl.

In some embodiments, R¹⁰ is n-propyl and R¹¹ is n-propyl.

In some embodiments, R¹⁰ is n-propyl and R¹¹ is isopropyl.

In some embodiments, R¹⁰ is n-propyl and R¹¹ is n-butyl.

In some embodiments, R¹⁰ is n-propyl and R¹¹ is sec-butyl.

In some embodiments, R¹⁰ is isopropyl and R¹¹ is isopropyl.

In some embodiments, R¹⁰ is isopropyl and R¹¹ is n-butyl.

In some embodiments, R¹⁰ is isopropyl and R¹¹ is sec-butyl.

In some embodiments, R¹⁰ is n-butyl and R¹¹ is n-butyl.

In some embodiments, R¹⁰ is n-butyl and R¹¹ is sec-butyl.

In some embodiments, R¹⁰ is sec-butyl and R¹¹ is sec-butyl.

In some embodiments, R¹⁰ is methyl.

In some embodiments, R¹⁰ is ethyl.

In some embodiments, R¹⁰ is propyl.

In some embodiments, R¹⁰ and R¹¹ are both not methyl.

In some embodiments, R¹⁰ is butyl.

In some embodiments, the compound is

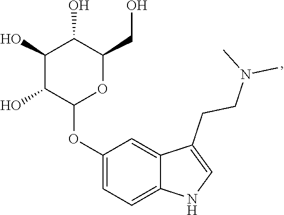 [SMILES: CN(C)CCc1c[nH]c2ccc(OC3O[C@H](CO)[C@@H](O)[C@H](O)[C@H]3O)cc12]

or a pharmaceutically acceptable salt thereof.

In some embodiments, the compound is

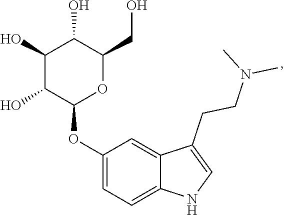 [SMILES: CN(C)CCc1c[nH]c2ccc(O[C@@H]3O[C@H](CO)[C@@H](O)[C@H](O)[C@H]3O)cc12]

or a pharmaceutically acceptable salt thereof.

In some embodiments, the compound is

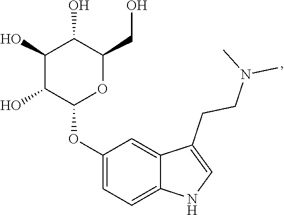 [SMILES: CN(C)CCc1c[nH]c2ccc(O[C@H]3O[C@H](CO)[C@@H](O)[C@H](O)[C@H]3O)cc12]

or a pharmaceutically acceptable salt thereof.

In some embodiments, the compound is

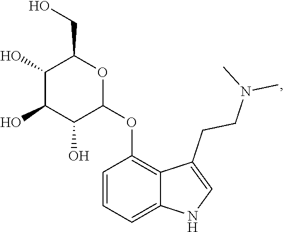 [SMILES: CN(C)CCc1c[nH]c2cccc(OC3O[C@H](CO)[C@@H](O)[C@H](O)[C@H]3O)c12]

or a pharmaceutically acceptable salt thereof.

In some embodiments, the compound is

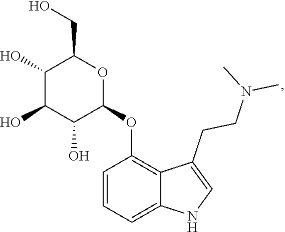 [SMILES: CN(C)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

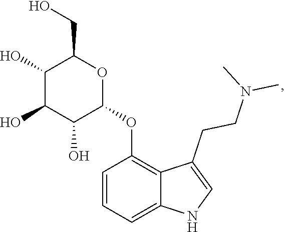 [SMILES: CN(C)CCc1c[nH]c2cccc(O[C@H]3O[C@H](CO)[C@@H](O)[C@H](O)[C@H]3O)c12]

or a pharmaceutically acceptable salt thereof.

In some embodiments, the compound is

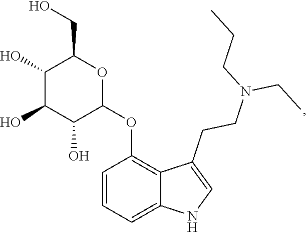 [SMILES: CCCN(CC)CCc1c[nH]c2cccc(OC3O[C@H](CO)[C@@H](O)[C@H](O)[C@H]3O)c12]

or a pharmaceutically acceptable salt thereof.

In some embodiments, the compound is

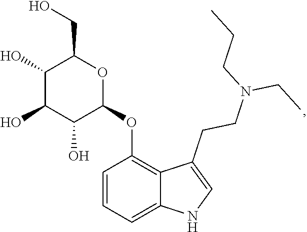 [SMILES: CCCN(CC)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

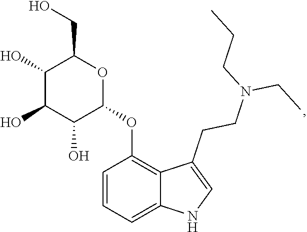 [SMILES: CCCN(CC)CCc1c[nH]c2cccc(O[C@H]3O[C@H](CO)[C@@H](O)[C@H](O)[C@H]3O)c12]

or a pharmaceutically acceptable salt thereof.

In some embodiments, the compound is

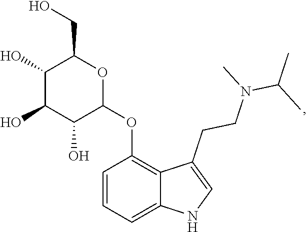 [SMILES: CC(C)N(C)CCc1c[nH]c2cccc(OC3O[C@H](CO)[C@@H](O)[C@H](O)[C@H]3O)c12]

or a pharmaceutically acceptable salt thereof.

In some embodiments, the compound is

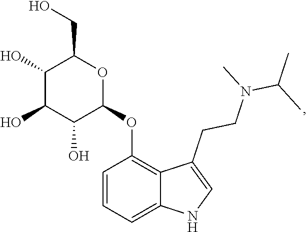 [SMILES: CC(C)N(C)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

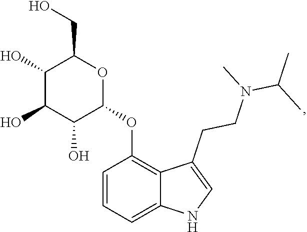 [SMILES: CC(C)N(C)CCc1c[nH]c2cccc(O[C@H]3O[C@H](CO)[C@@H](O)[C@H](O)[C@H]3O)c12]

or a pharmaceutically acceptable salt thereof.

In some embodiments, the compound is

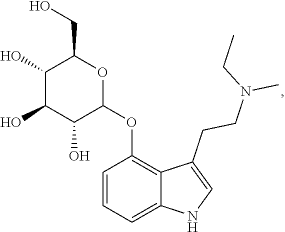 [SMILES: CCN(C)CCc1c[nH]c2cccc(OC3O[C@H](CO)[C@@H](O)[C@H](O)[C@H]3O)c12]

or a pharmaceutically acceptable salt thereof.

In some embodiments, the compound is

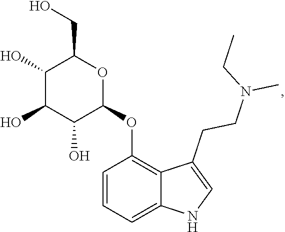 [SMILES: CCN(C)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

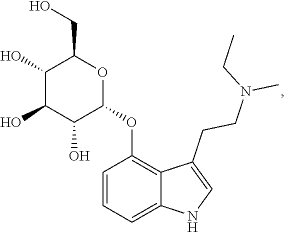 [SMILES: CCN(C)CCc1c[nH]c2cccc(O[C@H]3O[C@H](CO)[C@@H](O)[C@H](O)[C@H]3O)c12]

or a pharmaceutically acceptable salt thereof.

### Group R¹

In some embodiments of any of the aspects or embodiments described herein, R¹ is a glycosyl group.

The terms ““glycosylate”” or ““glycosy””, as used herein, refer to a saccharide group, such as a mono-, di-, tri-oligo- or a poly-saccharide group, which can be or has been bonded from its anomeric carbon either in the pyranose or furanose form, either in the a or the p conformation, or can be or has been bonded from a non-anomeric carbon atom, and can be in the pyranose or furanose form. The saccharide group can be bonded via an oxygen atom to another entity, the bonded saccharide group, inclusive of the oxygen atom, may be referred to herein as a “glycosyloxy” group, and can be said to be “O-glycosylated” or “O-linked”. Thus, the term glycosyl group, as used herein, includes glycosyloxy groups. Alternatively, the saccharide group may also be bonded from a carbon atom, and can then be said to be “C-glycosylated” or “C-linked”. Example monosaccharide groups include, but are not limited to, a pentosyl, a hexosyl, or a heptosyl group. Included in the term glycosyl are further stereoisomers, optical isomers, anomers, and epimers of the glycosyl group. Thus, a hexose group, for example, can be either an aldose or a ketose group, can be of D- or L-configuration, can assume either an a- or p-conformation, and can be a dextro- or levo-rotatory with respect to plane-polarized light.

Example glycosyl groups further include, glucosyl group, glucuronic acid group, a galactosyl group, a mannosyl group, a fucosyl group, a xylosyl group, a rhamnosyl group, a glucosaminyl group and a galactosaminyl group.

Glucose is usually present in solid form as a monohydrate with a closed pyran ring (dextrose hydrate). In aqueous solution, on the other hand, it is an open-chain to a small extent and is present predominantly as α- or β-pyranose, which interconvert. From aqueous solutions, the three known forms can be crystallized: a-glucopyranose, β-glucopyranose and β-glucopyranose hydrate. Glucose is a building block of the disaccharides lactose and sucrose (cane or beet sugar), of oligosaccharides such as raffinose and of polysaccharides such as starch, amylopectin, glycogen, and cellulose.

The open-chain form of glucose makes up less than 0.02% of the glucose molecules in an aqueous solution. The rest is one of two cyclic hemiacetal forms. In its open-chain form, the glucose molecule has an open (as opposed to cyclic) unbranched backbone of six carbon atoms, where C-1 is part of an aldehyde group H(C═O)—. Therefore, glucose is also classified as an aldose, or an aldohexose.

In solutions, the open-chain form of glucose (either “D-” or “L-”) exists in equilibrium with several cyclic isomers, each containing a ring of carbons closed by one oxygen atom. In aqueous solution, however, more than 99% of glucose molecules exist as pyranose forms. The open-chain form is limited to about 0.25%, and furanose forms exist in negligible amounts. The terms “glucose” and “D-glucose” are generally used for these cyclic forms as well. The ring arises from the open-chain form by an intramolecular nucleophilic addition reaction between the aldehyde group (at C-1) and either the C-4 or C-5 hydroxyl group, forming a hemiacetal linkage, —C(OH)H—O—.

The reaction between C-1 and C-5 yields a six-membered heterocyclic system called a pyranose, which is a monosaccharide sugar (hence “-ose”) containing a derivatised pyran skeleton. The (much rarer) reaction between C-1 and C-4 yields a five-membered furanose ring, named after the cyclic ether furan. In either case, each carbon in the ring has one hydrogen and one hydroxyl attached, except for the last carbon (C-4 or C-5) where the hydroxyl is replaced by the remainder of the open molecule (which is —(C(CH2OH)HOH)—H or —(CHOH)—H respectively).

The ring-closing reaction can give two products, denoted “α-” and “β-”. When a glucopyranose molecule is drawn in the Haworth projection, the designation “α-” means that the hydroxyl group attached to C-1 and the —CH2OH group at C-5 lies on opposite sides of the ring's plane (a trans arrangement), while “β-” means that they are on the same side of the plane (a cis arrangement). Therefore, the open-chain isomer D-glucose gives rise to four distinct cyclic isomers: α-D-glucopyranose, β-D-glucopyranose, α-D-glucofuranose, and β-D-glucofuranose. These five structures exist in equilibrium and interconvert, and the interconversion is much more rapid with acid catalysis.

In some aspects, R¹ is a glucosyl group.

In some embodiments, a glycosyl group is a monovalent substituent whose point of attachment is obtained by removing the hemiacetal hydroxyl group from the cyclic form of a monosaccharide and, by extension, of a lower oligosaccharide. In some embodiments, gylcosyl may be represented by

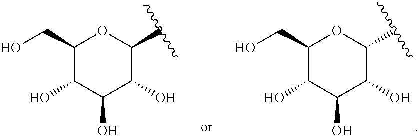 [SMILES: *[C@@H]1O[C@H](CO)[C@@H](O)[C@H](O)[C@H]1O ; *[C@H]1O[C@H](CO)[C@@H](O)[C@H](O)[C@H]1O]

In some alternative embodiments, a glycosyl group is a monovalent substituent whose point of attachment is obtained by removing a hydrogen from the cyclic form of a monosaccharide and, by extension, of a lower oligosaccharide. In some embodiments, gylcosyl may be represented by a substituent selected from the group consisting of

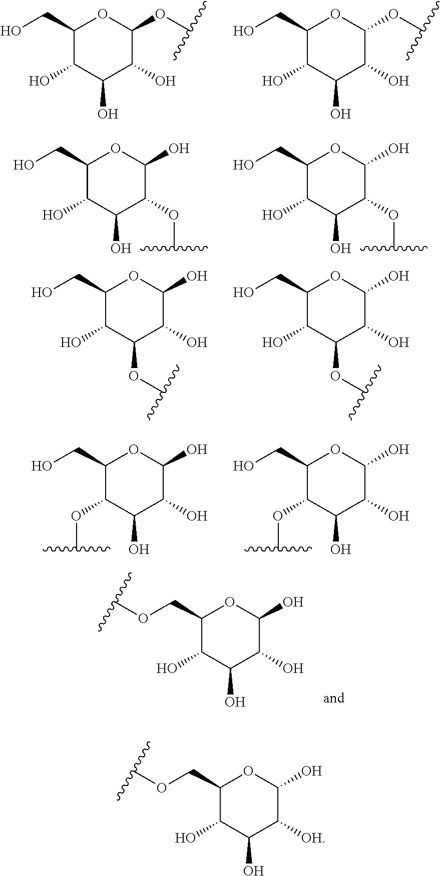

In some aspects, R¹ is

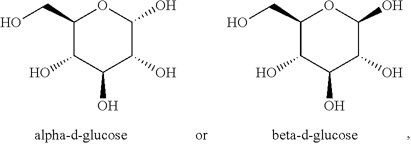 [SMILES: OC[C@H]1O[C@H](O)[C@H](O)[C@@H](O)[C@@H]1O ; OC[C@H]1O[C@@H](O)[C@H](O)[C@@H](O)[C@@H]1O]

linked through an oxygen atom.

Furthermore, continuing to the chemical compound having Formula (I) (and the other formulas described herein), the glycosyl groups in accordance herewith may be glycosyloxy groups (O-linked glycosyl groups) or C-linked glycosyl groups.

Continuing to refer to the chemical compound having Formula (I) (and the other formulas described herein), in some embodiments, the glycosyl group may be a D-glucosyl group, D-fructosyl group, D-mannosyl group, D-ribosyl group, D-talosyl group, D-lyxosyl group, D-allosyl group, D-altrosyl group, D-gulosyl group, D-idosyl group, N-acetyl-D-glucosaminyl group, N-acetyl-D-galactosaminyl group, D-quinovosyl group, D-maltosyl group, D-cellobiosyl group, D-lactosyl group, N-acetyl-D-lactosaminyl group, D-maltotiosyl group, D-glucuronic acid group, D-galactosyl group, D-mannosyl group, D-fucosyl group, D-xylosyl group, D-arabinosyl group, a D-rhamnosyl group, a D-glucosaminyl group, or a D-galactosaminyl group.

Continuing to refer to the chemical compound having formula (I), embodiments, the glycosyl group may be an L-glucosyl group, L-fructosyl group, L-mannosyl group, L-ribosyl group, L-talosyl group, L-lyxosyl group, L-allosyl group, L-altrosyl group, L-gulosyl group, L-idosyl group, N-acetyl-L-glucosaminyl group, N-acetyl-L-galactosaminyl group, L-quinovosyl group, L-maltosyl group, L-cellobiosyl group, L-lactosyl group, N-acetyl-L-lactosaminyl group, L-maltotiosyl group, L-glucuronic acid group, L-galactosyl group, L-mannosyl group, L-fucosyl group, L-xylosyl group, L-arabinosyl group, a L-rhamnosyl group, a L-glucosaminyl group, or a L-galactosaminyl group.

Continuing to refer to the chemical compound having formula (I), some embodiments, the glycosyl group may be a glycosyloxy group (i.e. a glycosyl group formed by bonding of the saccharide through its anomeric carbon atom). Thus, as will be clear, in some embodiments, the glycosyl group can be a glycosyloxy group selected from a glucosyloxy group, fructosyloxy group, mannosyoxy group, ribosyloxy group, talosyloxy group, lyxosyloxy group, allosyloxy group, altrosyloxy group, gulosyloxy group, idosyloxy group, N-acetyl-glucosaminyloxy group, N-acetyl-galactosaminyloxy group, quinovosyloxy group, maltosyloxy group, cellobiosyloxy group, lactosyloxy group, N-acetyl-lactosaminoxy group, maltotiosyloxy group, glucuronicoxy acid group, galactosyloxy group, mannosyloxy group fucosyloxy group, xylosyloxy group, arabinosyloxy group, a rhamnosyloxy group, a glucosaminoxy group, or a galactosaminoxy group.

In one aspect, provided herein is a pharmaceutically acceptable salt of a compound described herein (e.g., a compound of Formula (I), Formula (Ia), Formula (Ib), Formula (Ibb), Formula (Ic), Formula (Id), Formula (Ie), Formula (Ie-α), or Formula (Ie-β), or a subformula thereof).

### Methods of Making

As hereinbefore mentioned, the present disclosure relates to hydroxytryptamine compounds, such as 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT, and analogues or derivatives thereof (e.g., glycosylated analogues or derivatives). In general, the herein provided compositions exhibit functional properties which deviate from the functional properties of 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, or 5-HO-DMT. Thus, for example, the glycosylated 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT derivatives, can exhibit pharmacological properties which deviate from 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT. Furthermore, the glycosylated 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT derivatives may exhibit physico-chemical properties which differ from 44-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT. Thus, for example, glycosylated 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT derivatives may exhibit superior solubility in a solvent, for example, an aqueous solvent. The glycosylated 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT derivatives in this respect are useful in the formulation of pharmaceutical drug formulations. In one embodiment, the glycosylated 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT derivatives of the present disclosure can conveniently be biosynthetically produced. The practice of this method avoids the extraction of 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT and the performance of subsequent chemical reactions to achieve glycosylated derivatives. The method can efficiently yield substantial quantities of glycosylated 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT derivatives.

In some embodiments, provided herein is a process of preparing a compound of Formula (I):

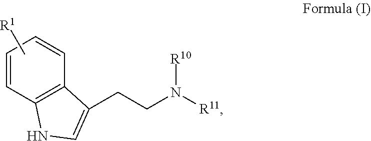

or a salt thereof, comprising:
glycosylating the hydroxy group of a compound of Formula (I-1):

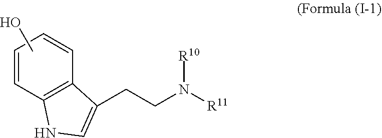

or a salt thereof, thereby producing the compound of Formula (I),
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl. In some embodiments, provided herein is a process of preparing a compound of Formula (Ia):

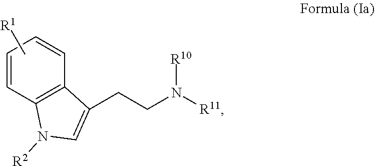

or a salt thereof, comprising:
glycosylating the hydroxy group of a compound of Formula (Ia-1):

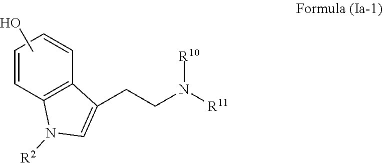

or a salt thereof, thereby producing the compound of Formula (Ia),
wherein
R¹ is a glycosyl group;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a process of preparing a compound of Formula (Ib):

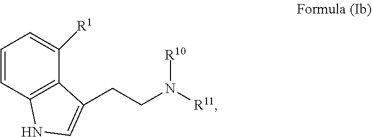

or a salt thereof, comprising:
glycosylating the hydroxy group of a compound of Formula (Ib-1):

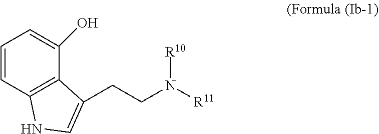

or a salt thereof, thereby producing the compound of Formula (Ib),
wherein
R¹ is a glycosyl group;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a process of preparing a compound of Formula (Ibb):

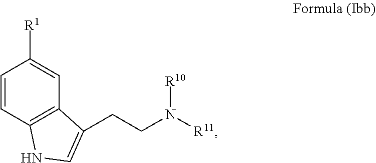

or a salt thereof, comprising:
glycosylating the hydroxy group of a compound of Formula (Ibb-1):

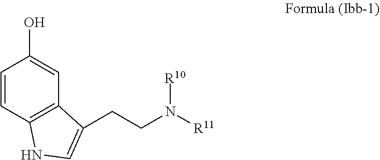

or a salt thereof, thereby producing the compound of Formula (Ibb),
wherein
R¹ is a glycosyl group;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a process of preparing a compound of Formula (Ic):

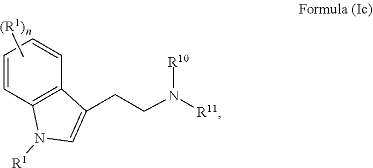

or a salt thereof, comprising:
glycosylating the hydroxy group of a compound of Formula (Ic-1):

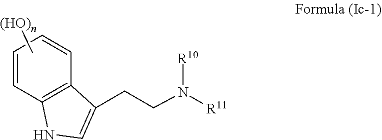

or a salt thereof, thereby producing the compound of Formula (Ic),
n is 1, 2, or 3;
R¹ is a glycosyl group, an —O-glycosyl group, or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl,
wherein at least one R¹ is a glycosyl group or an —O-glycosyl group.

In some embodiments, provided herein is a process of preparing a compound of Formula (Id):

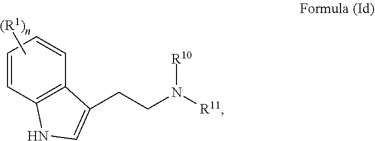

or a salt thereof, comprising:
glycosylating the hydroxy group of a compound of Formula (Id-1):

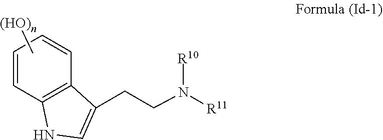

or a salt thereof, thereby producing the compound of Formula (Id),
wherein
n is 1, 2, or 3;
R¹ is a glycosyl group, an —O-glycosyl group, or —OH; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl,
wherein at least one R¹ is a glycosyl group or an —O-glycosyl group.

In some embodiments, provided herein is a process of preparing a compound of Formula (Ie):

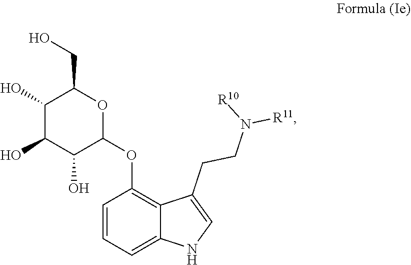

or a salt thereof, comprising:
glycosylating the hydroxy group of a compound of Formula (Ie-1):

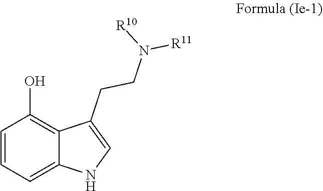

or a salt thereof, thereby producing the compound of Formula (Ie),
wherein
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a process of preparing a compound of Formula (Ie-α):

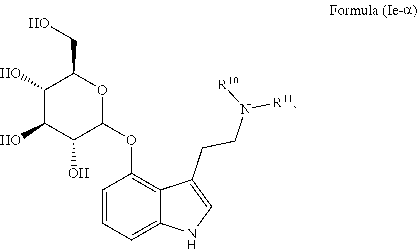

or a salt thereof, comprising:
glycosylating the hydroxy group of a compound of Formula (Ie-1):

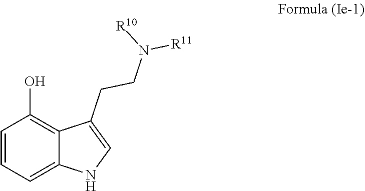

or a salt thereof, thereby producing the compound of Formula (Ie-α),
wherein
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, provided herein is a process of preparing a compound of Formula (Ie-β):

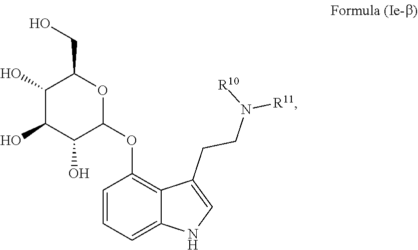

or a salt thereof, comprising:
glycosylating the hydroxy group of a compound of Formula (Ie-1):

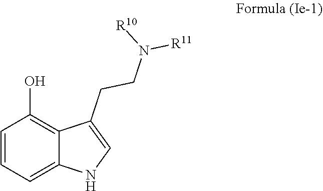

or a salt thereof, thereby producing the compound of Formula (Ie-β),
wherein
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

In some embodiments, R¹⁰ is selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl.

In some embodiments, R¹⁰ is selected from unsubstituted C₁-C₆ alkyl, unsubstituted C₂-C₆ alkenyl, and unsubstituted C₃-C₁₀ cycloalkyl.

In some embodiments, R¹⁰ is unsubstituted C₁-C₆ alkyl. In some embodiments, R¹⁰ is unsubstituted C₂-C₆ alkenyl. In some embodiments, R¹⁰ is unsubstituted C₃-C₁₀ cycloalkyl.

In some embodiments, R¹⁰ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

In some embodiments, R¹⁰ is methyl, ethyl, n-propyl, isopropyl, n-butyl, tert-butyl, sec-butyl, or iso butyl.

In some embodiments, R¹⁰ is methyl, ethyl, n-propyl, isopropyl, n-butyl, or sec-butyl.

In some embodiments, R¹⁰ is methyl. In some embodiments, R¹⁰ is ethyl. In some embodiments, R¹⁰ is n-propyl. In some embodiments, R¹⁰ is isopropyl. In some embodiments, R¹⁰ is n-butyl. In some embodiments, R¹⁰ is sec-butyl.

In some embodiments, R¹⁰ is ethenyl, 1-propenyl, 2-propenyl, 1-butenyl, 2-butenyl, or butadienyl.

In some embodiments, R¹⁰ is cyclopropyl, cyclobutyl, cyclopentyl, or cyclohexyl.

In some embodiments, R¹¹ is selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl.

In some embodiments, R¹¹ is selected from unsubstituted C₁-C₆ alkyl, unsubstituted C₂-C₆ alkenyl, and unsubstituted C₃-C₁₀ cycloalkyl.

In some embodiments, R¹¹ is unsubstituted C₁-C₆ alkyl. In some embodiments, R¹¹ is unsubstituted C₂-C₆ alkenyl. In some embodiments, R¹¹ is unsubstituted C₃-C₁₀ cycloalkyl.

In some embodiments, R¹¹ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

In some embodiments, R¹¹ is methyl, ethyl, n-propyl, isopropyl, n-butyl, tert-butyl, sec-butyl, or iso-butyl.

In some embodiments, R¹¹ is methyl, ethyl, n-propyl, isopropyl, n-butyl, or sec-butyl.

In some embodiments, R¹¹ is methyl. In some embodiments, R¹¹ is ethyl. In some embodiments, R¹¹ is n-propyl. In some embodiments, R¹¹ is isopropyl. In some embodiments, R¹¹ is n-butyl. In some embodiments, R¹¹ is sec-butyl.

In some embodiments, R¹¹ is ethenyl, 1-propenyl, 2-propenyl, 1-butenyl, 2-butenyl, or butadienyl.

In some embodiments, R¹¹ is cyclopropyl, cyclobutyl, cyclopentyl, or cyclohexyl. In some embodiments, R¹¹ is cyclopropyl.

In some embodiments, R¹⁰ is methyl and R¹¹ is methyl.

In some embodiments, R¹⁰ is methyl and R¹¹ is ethyl.

In some embodiments, R¹⁰ is methyl and R¹¹ is n-propyl.

In some embodiments, R¹⁰ is methyl and R¹¹ is isopropyl.

In some embodiments, R¹⁰ is methyl and R¹¹ is n-butyl.

In some embodiments, R¹⁰ is methyl and R¹¹ is sec-butyl.

In some embodiments, R¹⁰ is ethyl and R¹¹ is ethyl.

In some embodiments, R¹⁰ is ethyl and R¹¹ is n-propyl.

In some embodiments, R¹⁰ is ethyl and R¹¹ is isopropyl.

In some embodiments, R¹⁰ is ethyl and R¹¹ is n-butyl.

In some embodiments, R¹⁰ is ethyl and R¹¹ is sec-butyl.

In some embodiments, R¹⁰ is n-propyl and R¹¹ is n-propyl.

In some embodiments, R¹⁰ is n-propyl and R¹¹ is isopropyl.

In some embodiments, R¹⁰ is n-propyl and R¹¹ is n-butyl.

In some embodiments, R¹⁰ is n-propyl and R¹¹ is sec-butyl.

In some embodiments, R¹⁰ is isopropyl and R¹¹ is isopropyl.

In some embodiments, R¹⁰ is isopropyl and R¹¹ is n-butyl.

In some embodiments, R¹⁰ is isopropyl and R¹¹ is sec-butyl.

In some embodiments, R¹⁰ is n-butyl and R¹¹ is n-butyl.

In some embodiments, R¹⁰ is n-butyl and R¹¹ is sec-butyl.

In some embodiments, R¹⁰ is sec-butyl and R¹¹ is sec-butyl.

In some embodiments, R¹⁰ and R¹¹ are both not methyl.

In some embodiments, R¹⁰ and R¹¹, together with the nitrogen atom to which they are attached, form a substituted 3- to 10-membered heterocyclyl or an unsubstituted 3- to 10-membered heterocyclyl.

In some embodiments, R¹⁰ and R¹¹, together with the nitrogen atom to which they are attached, form an unsubstituted 3- to 10-membered heterocyclyl.

In some embodiments, R¹⁰ and R¹¹, together with the nitrogen atom to which they are attached, form cyclopropyl, cyclobutyl, cyclopentyl, or cyclohexyl. In some embodiments, R¹⁰ and R¹¹, together with the nitrogen atom to which they are attached, form cyclopropyl. In some embodiments, R¹⁰ and R¹¹, together with the nitrogen atom to which they are attached, form cyclopentyl.

In some embodiments, the percent yield of the compound of Formula (I), Formula (Ia), Formula (Ib), Formula (Ibb), Formula (Ic), Formula (Ie), Formula (Ie-α), or Formula (Ie-β), or a subformula thereof is greater than 85%, is greater than 90%, is greater than 93%, or is greater than 95%. In some embodiments, the percent yield of the compound of Formula (I), Formula (Ia), Formula (Ib), Formula (Ibb), Formula (Ic), Formula (Ie), Formula (Ie-α), or Formula (Ie-β), or a subformula thereof is greater than 75%, is greater than 80%, is greater than 85%, is greater than 90%, is greater than 91%, is greater than 92%, is greater than 93%, is greater than 94%, is greater than 95%, is greater than 96%, is greater than 97%, is greater than 98%, or is greater than 99%. In some embodiments, glycosylating the hydroxy group results in a percent yield of the compound of Formula (I), Formula (Ia), Formula (Ib), Formula (Ibb), Formula (Ic), Formula (Ie), Formula (Ie-α), or Formula (Ie-β), or a subformula thereof that is greater than 75%, is greater than 80%, is greater than 85%, is greater than 90%, is greater than 91%, is greater than 92%, is greater than 93%, is greater than 94%, is greater than 95%, is greater than 96%, is greater than 97%, is greater than 98%, or is greater than 99%.

In some embodiments, the compound is

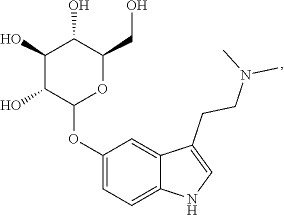 [SMILES: CN(C)CCc1c[nH]c2ccc(OC3O[C@H](CO)[C@@H](O)[C@H](O)[C@H]3O)cc12]

or a pharmaceutically acceptable salt thereof.

In some embodiments, the compound is

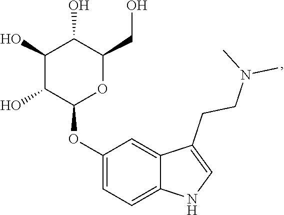 [SMILES: CN(C)CCc1c[nH]c2ccc(O[C@@H]3O[C@H](CO)[C@@H](O)[C@H](O)[C@H]3O)cc12]

or a pharmaceutically acceptable salt thereof.

In some embodiments, the compound is

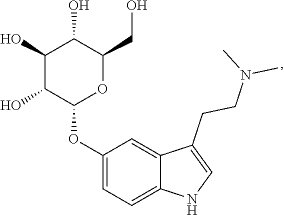 [SMILES: CN(C)CCc1c[nH]c2ccc(O[C@H]3O[C@H](CO)[C@@H](O)[C@H](O)[C@H]3O)cc12]

or a pharmaceutically acceptable salt thereof.

In some embodiments, the compound is

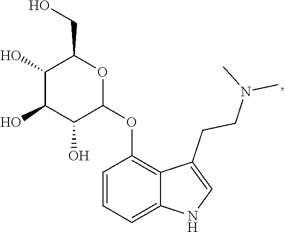 [SMILES: CN(C)CCc1c[nH]c2cccc(OC3O[C@H](CO)[C@@H](O)[C@H](O)[C@H]3O)c12]

or a pharmaceutically acceptable salt thereof.

In some embodiments, the compound is

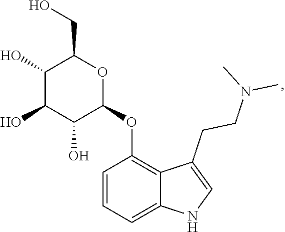 [SMILES: CN(C)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

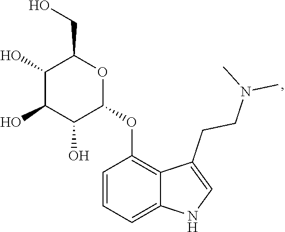 [SMILES: CN(C)CCc1c[nH]c2cccc(O[C@H]3O[C@H](CO)[C@@H](O)[C@H](O)[C@H]3O)c12]

or a pharmaceutically acceptable salt thereof.

In some embodiments, the compound is

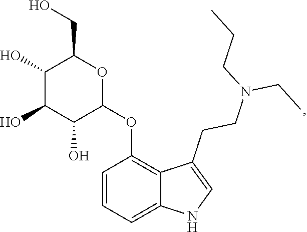 [SMILES: CCCN(CC)CCc1c[nH]c2cccc(OC3O[C@H](CO)[C@@H](O)[C@H](O)[C@H]3O)c12]

or a pharmaceutically acceptable salt thereof.

In some embodiments, the compound is

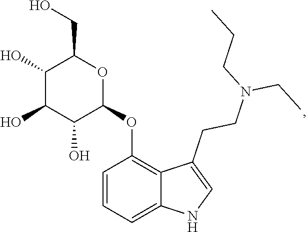 [SMILES: CCCN(CC)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

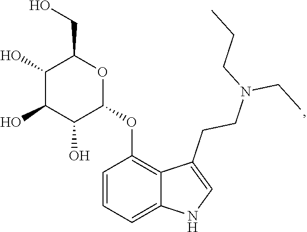 [SMILES: CCCN(CC)CCc1c[nH]c2cccc(O[C@H]3O[C@H](CO)[C@@H](O)[C@H](O)[C@H]3O)c12]

or a pharmaceutically acceptable salt thereof.

In some embodiments, the compound is

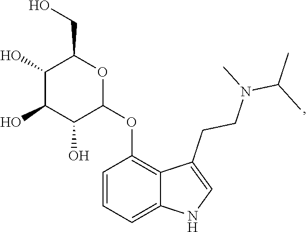 [SMILES: CC(C)N(C)CCc1c[nH]c2cccc(OC3O[C@H](CO)[C@@H](O)[C@H](O)[C@H]3O)c12]

or a pharmaceutically acceptable salt thereof.

In some embodiments, the compound is

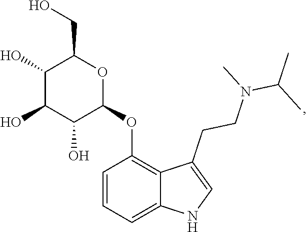 [SMILES: CC(C)N(C)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

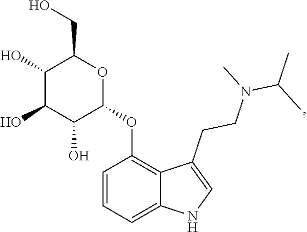 [SMILES: CC(C)N(C)CCc1c[nH]c2cccc(O[C@H]3O[C@H](CO)[C@@H](O)[C@H](O)[C@H]3O)c12]

or a pharmaceutically acceptable salt thereof.

In some embodiments, the compound is

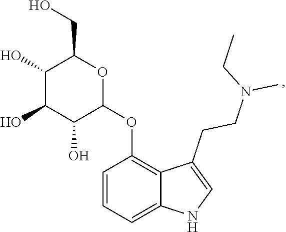 [SMILES: CCN(C)CCc1c[nH]c2cccc(OC3O[C@H](CO)[C@@H](O)[C@H](O)[C@H]3O)c12]

or a pharmaceutically acceptable salt thereof.

In some embodiments, the compound is

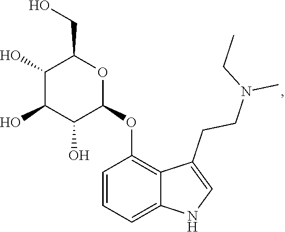 [SMILES: CCN(C)CCc1c[nH]c2cccc(O[C@@H]3O[C@H](CO)[C@@H](O)[C@H](O)[C@H]3O)c12]

or a pharmaceutically acceptable salt thereof.

In some embodiments, the compound is

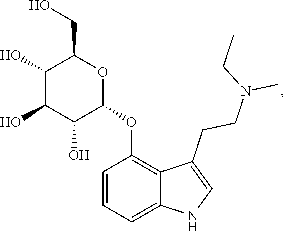 [SMILES: CCN(C)CCc1c[nH]c2cccc(O[C@H]3O[C@H](CO)[C@@H](O)[C@H](O)[C@H]3O)c12]

or a pharmaceutically acceptable salt thereof.

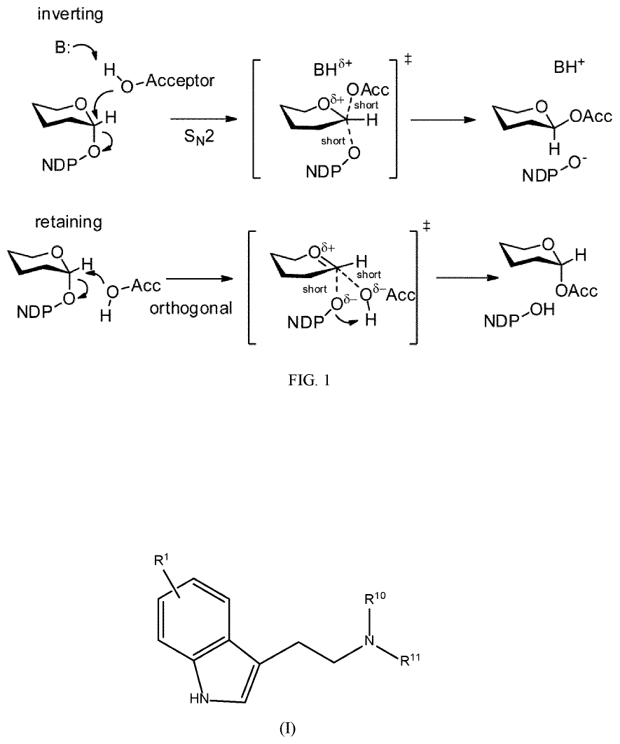

In certain embodiments, glycosylated 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, and 5-HO-DMT derivatives are prepared by glycosyltransferases (GTFs). Glycosyltransferases are enzymes that establish glycosidic linkages. They catalyze the transfer of saccharide moieties, such as glucose, from a nucleotide sugar (also known as the “glycosyl donor” or glycosyl compound) to a nucleophilic glycosyl acceptor molecule, the nucleophile of which can be oxygen-carbon-, nitrogen-, or sulfur-based. Glycosyltransferases can be segregated into “retaining” or “inverting” enzymes according to whether the stereochemistry of the donor's anomeric bond is retained (α→α) or inverted (α→β) during the transfer. See FIG. 1. The inverting mechanism is straightforward, requiring a single nucleophilic attack from the accepting atom to invert stereochemistry.

In certain embodiments, glycosylated 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, or 5-HO-DMT derivatives are prepared by UDP-glycosyltransferase (UGT), a class of enzymes. In certain embodiments, glycosylated 4-HO-MET, 4-HO-MET, 4-HO-MPT, 4-HO-MiPT, 4-HO-McPeT, 4-HO-MALT, 4-HO, DALT, 4-HO-DET, 4-HO-EPT, 4-HO-DPT, 4-HO-PiPT, 4-HO-DiPT, 4-HO-DBT, 4-HO-DSBT, 4-HO-pyr-T, 4-HO-DMT, or 5-HO-DMT derivatives are prepared by UDP-glycosyltransferase (UGT), a class of enzymes, described herein.

The result of glycosyl transfer can be a carbohydrate, glycoside, oligosaccharide, or a polysaccharide.

In accordance herewith, any glycosyl compound may be selected, obtained or prepared and used. Suitable glycosyl compounds include, mono-saccharides, disaccharides, tri-saccharides and poly-saccharides.

In an certain embodiments, glycosyl compounds which may be selected are glucose and glucosyl containing compounds and glucose and glucosyl derivatives, such as uridine diphosphate glucose (UDP-glucose).

In a further example embodiment, glycosyl compounds which may be selected are glucuronic acid and derivatives thereof, such as UDP-glucuronic acid.

In a further example embodiment, glycosyl compounds which may be selected are galactose and galactosyl containing compounds and galactose and galactosyl derivatives, such as uridine diphosphate galactose (UDP-galactose). In a further example embodiment, glycosyl compounds which may be selected are mannose and derivatives thereof, such as GDP-mannose.

In a further example embodiment, glycosyl compounds which may be selected are fucose and fucosyl containing compounds and fucose and fucosyl derivatives, such as GDP-frucose.

In a further example embodiment, glycosyl compounds which may be selected are xylose and derivatives thereof, such as UDP-xylose.

In a yet further example embodiment, glycosyl compounds which may be selected are and rhamnose and derivatives thereof, UPD-rhamnose.

In one example embodiment, the reaction may be catalyzed by a glucosyl transferase. An example chemical reaction catalyzed by a UDP glycosyl transferase wherein the glucose moiety of UDP-glucose is transferred to 4-OH-MET in a chemical reaction which results in the formation of a glycosidic bond, and which is catalyzed by a UDP glycosyl transferase. Thus, in one embodiment, the glycosylated 4-OH-MET derivative can be formed in a reaction between a UDP-glycosyl compound and 4-OH-MET, wherein the hydroxy group reacts with the glycosyl group of the UDP-glycosyl compound to form a glycosidic bond, and wherein the reaction is catalyzed by the UDP-glycosyl transferase.

In some embodiments, glycosylating the hydroxy group of the compound of Formula (I-1), Formula (Ia-1), Formula (Ib-1), Formula (Ibb-1), Formula (Ic-1), Formula (Id-1), or Formula (Ie-1) comprises contacting the compound of Formula (I-1), Formula (Ia-1), Formula (Ib-1), Formula (Ibb-1), Formula (Ic-1), Formula (Id-1), or Formula (Ie-1) with UDP-glucose, UDP-galactose, UDP-xylose, UDP-glucuronic acid, GDP-mannose, or GDP-fucose.

Thus, for example, a nucleic acid sequence encoding a glycosyl transferase may be introduced into a host cell, and upon cell growth the host cells can make the glycosyl transferase.

Typically, a nucleic acid sequence encoding a glycosyl transferase further includes one or more additional nucleic acid sequences, for example, a nucleic acid sequences controlling expression of the glycosyl transferase, and these one or more additional nucleic acid sequences together with the nucleic acid sequence encoding the glycosyl transferase can be said to form a chimeric nucleic acid sequence.

A host cell which upon cultivation expresses the chimeric nucleic acid can be selected and used in accordance with the present disclosure. Suitable host cells in this respect include, for example, microbial cells, such as bacterial cells, yeast cells, for example, and algal cells or plant cells. A variety of techniques and methodologies to manipulate host cells to introduce nucleic acid sequences in cells and attain expression exists and are well known to the skilled artisan. These methods include, for example, cation based methods, for example, lithium ion or calcium ion based methods, electroporation, biolistics, and glass beads based methods. As will be known to those of skill in the art, depending on the host cell selected, the methodology to introduce nucleic acid material in the host cell may vary, and, furthermore, methodologies may be optimized for uptake of nucleic acid material by the host cell, for example, by comparing uptake of nucleic acid material using different conditions. Detailed guidance can be found, for example, in Sambrook et al., Molecular Cloning, a Laboratory Manual, Cold Spring Harbor Laboratory Press, 2012, Fourth Ed. It is noted that the chimeric nucleic acid is a non-naturally occurring chimeric nucleic acid sequence and can be said to be heterologous to the host cell.

### Glycosyltransferases

In some embodiments, glycosylating the hydroxy group of the compound of Formula (I-1), Formula (Ia-1), Formula (Ib-1), Formula (Ibb-1), Formula (Ic-1), Formula (Id-1), or Formula (Ie-1) comprises contacting the compound of Formula (I-1), Formula (Ia-1), Formula (Ib-1), Formula (Ibb-1), Formula (Ic-1), Formula (Id-1), or Formula (Ie-1) with an enzyme. In some embodiments, glycosylating the hydroxy group of the compound of Formula (I-1), Formula (Ia-1), Formula (Ib-1), Formula (Ibb-1), Formula (Ic-1), Formula (Id-1), or Formula (Ie-1) comprises contacting the compound of Formula (I-1), Formula (Ia-1), Formula (Ib-1), Formula (Ibb-1), Formula (Ic-1), Formula (Id-1), or Formula (Ie-1) with a glycosyltransferase.

In some embodiments, the glycosyltransferase is encoded by a nucleic acid sequence that is at least 70% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 75% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 80% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 85% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 90% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 95% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 96% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 97% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 98% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 99% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence that is at least 100% identical to any one of SEQ ID NOs: 1, 3, 5, 7, 9, 11, 13, 15, 17, 19, 21, 23, 25, and 27.

In some embodiments, the glycosyltransferase is encoded by a nucleic acid sequence of any one of SEQ ID NOs: 1, 3, 5, 7, 9, 11, 13, 15, 17, 19, 21, 23, 25, and 27.

SEQ ID NO: 1: GLY70 gene; GLY70-At71C2
ATGGCCAAACAGCAAGAAGCAGAACTGATTTTTATCCCGTTTCCGATTCCGGGTCATATT
CTGGCAACCATTGAACTGGCAAAACGTCTGATTAGCCATCAGCCGAGCCGTATTCATACC
ATTACCATTCTGCATTGGAGCCTGCCGTTTCTGCCGCAGAGCGATACCATTGCATTTCTG
AAAAGCCTGATTGAAACCGAAAGTCGTATTCGCCTGATTACCCTGCCGGATGTTCAGAAT
CCGCCTCCGATGGAACTGTTTGTTAAAGCAAGCGAAAGCTACATCCTGGAATACGTGAAA
AAAATGGTTCCGCTGGTTCGTAATGCACTGAGCACCCTGCTGAGCAGCCGTGATGAAAGC
GATAGCGTTCATGTTGCAGGTCTGGTTCTGGATTTTTTCTGTGTTCCGCTGATTGATGTG
GGCAATGAATTTAATCTGCCGAGCTATATCTTTCTGACCTGTAGCGCAAGCTTTCTGGGT
ATGATGAAATATCTGCTGGAACGTAATCGTGAAACCAAACCGGAACTGAATCGTAGCAGT
GATGAAGAAACCATTAGCGTTCCGGGTTTTGTTAATAGCGTGCCGGTTAAAGTTCTGCCT
CCGGGTCTGTTTACCACCGAAAGCTATGAAGCATGGGTTGAAATGGCAGAACGTTTTCCG
GAAGCAAAAGGTATTCTGGTGAATAGCTTTGAAAGCCTGGAACGCAATGCCTTCGATTAT
TTTGATCGTCGTCCGGATAACTATCCGCCTGTTTATCCGATTGGTCCGATTCTGTGTAGC
AATGATCGTCCGAATCTGGATCTGAGCGAACGTGATCGTATTCTGAAATGGCTGGATGAT
CAGCCGGAAAGCAGCGTTGTGTTTCTGTGCTTTGGTAGCCTGAAAAGTCTGGCAGCAAGC
CAGATTAAAGAAATTGCACAGGCACTGGAACTGGTGGGTATTCGTTTTCTGTGGTCAATT
CGTACCGATCCGAAAGAATATGCAAGCCCGAATGAAATTCTGCCTGATGGTTTTATGAAT
CGCGTTATGGGTCTGGGTTTAGTTTGTGGTTGGGCACCGCAGGTTGAAATCCTGGCACAT
AAAGCAATTGGTGGTTTTGTGAGCCATTGCGGTTGGAATAGCATTCTGGAAAGTCTGCGT
TTTGGTGTTCCGATTGCAACCTGGCCGATGTATGCAGAACAGCAGCTGAATGCATTTACC
ATCGTTAAAGAATTAGGTCTGGCCCTGGAAATGCGTCTGGATTATGTTAGCGAATATGGC
GAAATTGTGAAAGCCGATGAAATTGCCGGTGCAGTTCGTAGCCTGATGGATGGTGAAGAT
GTTCCGCGTCGTAAACTGAAAGAAATCGCAGAAGCAGGTAAAGAAGCAGTTATGGATGGC
GGTAGCAGCTTTGTTGCAGTTAAACGTTTTATTGATGGCCTGTAA
SEQ ID NO: 3: GLY73 gene; GLY73-Qs88D_2
ATGGAAGATGCCATTGTTCTGTATCCGAGTCCTGGTCGTGGTCATCTGATTAGCATGGTT
GAACTGGGTAAACTGATCCTGAAAAATCATCCGAGCTTTAGCATTACCATTCTGATTCTG
AGCGAACCGAATACCAATGCAAATAGCACCGCACCGAATTATGTTGCAAGCAGCACCACC
GAATATATCACCACCGTTAATAGCACAACCCCGAGCATTACCTTTCATCATCTGCCTCCG
ATTAGCGAAGTTCCGCCTAGCACCAGCAGCTTTGTTGAACTGAGCTATCTGATTCCGCGT
TTTAACAATCAGAACCTGCATCAGATTCTGAAAACCATTAGCCAGACCAGCAAACTGAAA
GCCTTTATCATCGATTTTTTCTGCGACGCAGCATTTGAAGTTGCAACCAATCTGGATATT
CCGACCTATTATTTCTTTACCAGCGGTGCATGTTATCTGGCAGCACTGCTGTATCGTCCG
ACACTGCATAAAAAAGTTGACAAAAGCCTGAAAGATCTGGGTAATACCCTGCTGGACATT
CCGGGTCTGCCACCGATTCAGGCAAGCGATCTGCCGGAACCGTGGAAAGATCGTACCAGC
AAAGAATATCAGTATAGCCTGAATGTTGCAGCCCATATGGCAAAAAGCAATGGTCTGCTG
GTTAATACCTTTGATCTGCTGGAAACCAAAGCCATTAAAGCAATTAGCGGTGGTCTGTGT
GTTCCGGATCGTACAACCCCTCCGATTTTTTGTATTGGTCCGCTGATTAGCGATAGCAAT
CAGGATGTTGAAGATCATGAATGTCTGAATTGGCTGAATAGCCAGCCGAGCCGTAGCGTT
GTTTTTCTGTGTTTTGGTAGCCTGGGTCTGTTTAGTGCAAAACAGCTGAAAGAAATTGCA
GTTGGTCTGGAAAATAGCGGTCAGCGTTTTCTGTGGGTTCTGCGTAATCCTCCGAGCGAT
AATGATAAAGATCCGAATCTGGATGAACTGCTGCCGAAAGGTTTTCTGGAACGTACCAAA
GAAAAAGGCTTTGTTGTTAAACAGTGGGCACCGCAGGTTGCAGTTCTGCGTCATGATAGC
GTTGGTGGTTTTGTTACCCATTGTGGTTGGAATAGCGTTCTGGAAGCAGTTTGGTGTCGT
GTTCCGATGGTTGGTTGGCCTCTGTATGCAGAACAGCGTCTGAATAGCGTGGTTCTGGTT
GAAGAAATTAAACTGGCAAGCGGTCTGAATGAAAGCGAAGATGGTCTGGTTAGCGCAGCA
GAACTGGAAAAACGTGTTAAAGAACTGATGGATAGTGAAGTTGGCACCGAAGTTCGTGAA
CGTGTTAGCGGTCTGCGTGATGAAGCAGTTACCGCAGTTAAAGAAGGTGGTAGCAGCCAC
GTTGCACTGGCAAAACTGGCAGAACTGTGGCGTCAGAATTAA
SEQ ID NO: 5: GLY74 gene; GLY74-At71D1
ATGCGTAATGTGGAACTGATTTTTATCCCGACACCGACCGTTGGTCATCTGGTTCCGTTT
CTGGAATTTGCACGTCGTCTGATTGAACAGGATGATCGTATTCGTATTACCATCCTGCTG
ATGAAACTGCAGGGTCAGAGCCATCTGGATACCTATGTTAAAAGCATTGCAAGCAGCCAG
CCGTTTGTTCGTTTTATTGATGTGCCGGAACTGGAAGAAAAACCGACACTGGGTAGCACC
CAGAGCGTTGAAGCATATGTTTATGATGTGATTGAACGCAATATTCCGCTGGTGCGTAAT
ATTGTTATGGATATTCTGACCAGCCTGGCACTGGATGGTGTTAAAGTTAAAGGTCTGGTT
GTGGATTTTTTCTGCCTGCCGATGATTGATGTTGCCAAAGATATTAGCCTGCCGTTTTAT
GTTTTTCTGACCACCAATAGCGGTTTTCTGGCAATGATGCAGTATCTGGCAGATCGTCAT
AGCCGTGATACCAGCGTTTTTGTTCGTAATAGCGAAGAAATGCTGAGCATTCCGGGTTTT
GTTAATCCGGTTCCGGCAAATGTTCTGCCGAGCGCACTGTTTGTTGAAGATGGTTATGAT
GCGTATGTTAAACTGGCCATCCTGTTTACCAAAGCCAATGGTATTCTGGTGAATAGCAGC
TTTGATATCGAACCGTATAGCGTGAATCACTTTCTGCAAGAACAGAATTATCCGAGCGTT
TATGCAGTTGGTCCGATCTTTGATCTGAAAGCACAGCCGCATCCGGAACAGGATCTGACC
CGTCGTGATGAACTGATGAAATGGCTGGATGATCAGCCGGAAGCAAGCGTTGTGTTTCTG
TGTTTTGGTAGCATGGCACGTCTGCGTGGTAGCCTGGTTAAAGAAATTGCACATGGTCTG
GAACTGTGCCAGTATCGTTTTCTGTGGTCACTGCGTAAAGAAGAAGTTACCAAAGACGAC
CTGCCGGAAGGCTTTCTGGATCGTGTTGATGGTCGTGGTATGATTTGTGGTTGGAGTCCG
CAGGTTGAAATTCTGGCACATAAAGCAGTTGGTGGTTTTGTGAGCCATTGCGGTTGGAAT
AGCATTGTTGAAAGCCTGTGGTTTGGTGTTCCGATTGTTACCTGGCCGATGTATGCAGAA
CAGCAGCTGAATGCATTTCTGATGGTGAAAGAACTGAAACTGGCAGTTGAACTGAAGCTG
GATTATCGTGTTCATTCCGATGAAATTGTGAACGCCAATGAAATTGAAACCGCCATTCGT
TATGTGATGGATACCGATAACAATGTTGTGCGTAAACGTGTCATGGATATCAGCCAGATG
ATTCAGCGTGCAACCAAAAATGGTGGTAGCAGTTTTGCAGCCATCGAGAAATTTATCTAT
GACGTGATTGGCATCAAGCCGTAA
SEQ ID NO: 7: GLY77 gene; GLY77-Sr71E1
ATGAGCACCAGCGAACTGGTTTTTATTCCGAGTCCTGGTGCAGGTCATCTGCCTCCGACC
GTTGAACTGGCAAAACTGCTGCTGCATCGTGATCAGCGTCTGAGCGTTACCATTATTGTT
ATGAATCTGTGGCTGGGTCCGAAACATAATACCGAAGCACGTCCGTGTGTTCCGAGCCTG
CGTTTTGTTGATATTCCGTGTGATGAAAGCACCATGGCACTGATTAGCCCGAATACCTTT
ATTAGCGCATTTGTGGAACATCATAAACCGCGTGTTCGTGATATTGTGCGTGGTATTATT
GAAAGCGATAGCGTTCGTCTGGCAGGTTTTGTTCTGGATATGTTTTGTATGCCGATGAGT
GATGTGGCCAATGAATTTGGTGTGCCGAGCTATAACTATTTTACCAGCGGTGCAGCAACC
CTGGGTCTGATGTTTCATCTGCAGTGGAAACGTGATCATGAAGGTTATGATGCAACCGAA
CTGAAAAATAGCGATACCGAACTGTCAGTTCCGAGCTATGTTAATCCGGTTCCGGCAAAA
GTTCTGCCTGAAGTTGTGCTGGATAAAGAAGGTGGTAGCAAAATGTTTCTGGATCTGGCA
GAACGTATTCGTGAAAGCAAAGGCATTATTGTGAATAGCTGTCAGGCAATTGAACGTCAT
GCACTGGAATATCTGAGCAGCAATAACAATGGTATTCCGCCTGTTTTTCCGGTTGGTCCG
ATTCTGAATCTGGAAAACAAAAAAGATGATGCCAAAACCGATGAAATTATGCGCTGGCTG
AATGAACAGCCGGAAAGCAGCGTTGTTTTTCTGTGTTTTGGTAGCATGGGCAGCTTTAAT
GAGAAACAGGTTAAAGAAATTGCCGTGGCCATTGAACGTAGCGGTCATCGTTTTCTGTGG
TCACTGCGTCGTCCGACACCGAAAGAAAAAATTGAATTTCCGAAAGAATATGAGAACCTG
GAAGAAGTGCTGCCGGAAGGTTTTCTGAAACGTACCAGCAGCATTGGTAAAGTTATTGGT
TGGGCACCGCAGATGGCAGTTCTGAGCCATCCGAGCGTTGGTGGTTTTGTTAGCCATTGT
GGTTGGAATAGCACCCTGGAAAGCATGTGGTGTGGTGTTCCGATGGCAGCATGGCCTCTG
TATGCAGAACAGACCCTGAATGCATTTCTGCTGGTTGTTGAATTAGGTCTGGCAGCCGAA
ATTCGTATGGATTATCGTACCGATACCAAAGCAGGCTATGATGGTGGTATGGAAGTTACC
GTTGAAGAAATTGAAGATGGCATTCGCAAACTGATGTCAGATGGTGAAATTCGCAACAAA
GTGAAGGACGTGAAAGAGAAAAGTCGCGCAGCAGTTGTTGAAGGTGGTTCAAGCTATGCA
AGTATCGGCAAATTCATCGAACATGTTAGCAACGTGACCATTTAA
SEQ ID NO: 9: GLY87 gene; GLY87-At71C1_Sr71E1_354
ATGGGCAAACAAGAAGATGCCGAACTGGTTATTATTCCGTTTCCGTTTAGCGGTCATATT
CTGGCAACCATTGAACTGGCAAAACGTCTGATTAGCCAGGATAATCCGCGTATTCATACC
ATTACCATTCTGTATTGGGGTCTGCCGTTTATTCCGCAGGCAGATACCATTGCATTTCTG
CGTAGCCTGGTTAAAAATGAACCGCGTATCCGTCTGGTTACCCTGCCGGAAGTTCAGGAT
CCGCCTCCGATGGAACTGTTTGTTGAATTTGCAGAAAGCTATATCCTGGAATATGTGAAA
AAAATGGTGCCGATTATTCGTGAAGCACTGAGCACCCTGCTGAGCAGCCGTGATGAAAGC
GGTAGCGTTCGTGTTGCAGGTCTGGTTCTGGATTTTTTCTGTGTTCCGATGATTGATGTG
GGCAACGAATTTAATCTGCCGAGCTATATCTTTCTGACCTGTAGCGCAGGTTTTCTGGGT
ATGATGAAATATCTGCCGGAACGTCATCGTGAAATCAAAAGCGAATTTAACCGCAGCTTT
AACGAAGAACTGAATCTGATTCCGGGTTATGTTAATAGCGTTCCGACCAAAGTGCTGCCG
AGCGGTCTGTTTATGAAAGAAACCTATGAACCGTGGGTAGAACTGGCCGAACGTTTTCCG
GAAGCAAAAGGTATTCTGGTTAATAGCTATACCGCACTGGAACCGAATGGCTTCAAATAT
TTCGATCGTTGTCCGGATAACTACCCGACCATTTATCCGATTGGTCCGATTCTGAATCTG
GAAAACAAAAAAGATGATGCCAAAACCGATGAAATTATGCGCTGGCTGAATGAACAGCCG
GAAAGCAGCGTTGTGTTTCTGTGCTTTGGTAGCATGGGTAGCTTTAATGAAAAACAGGTG
AAAGAAATTGCCGTGGCAATTGAACGTAGTGGTCATCGTTTTCTGTGGTCACTGCGTCGT
CCGACACCGAAAGAAAAAATTGAATTTCCGAAAGAATATGAGAACCTGGAAGAAGTTCTG
CCTGAAGGCTTTCTGAAACGTACCAGCAGCATTGGTAAAGTTATTGGTTGGGCACCGCAG
ATGGCAGTTCTGAGCCATCCGAGCGTTGGTGGTTTTGTTAGCCATTGTGGTTGGAATAGC
ACCCTGGAAAGCATGTGGTGTGGTGTGCCGATGGCAGCATGGCCTCTGTATGCAGAACAG
ACCCTGAATGCCTTTCTGCTGGTTGTTGAACTGGGTTTAGCAGCAGAAATTCGTATGGAT
TATCGTACCGATACCAAAGCCGGTTATGATGGTGGTATGGAAGTTACCGTTGAAGAAATT
GAAGATGGCATTCGCAAACTGATGAGTGATGGTGAAATTCGCAACAAAGTGAAGGATGTC
AAAGAAAAATCACGTGCAGCAGTTGTTGAAGGTGGTAGCAGCTATGCAAGTATTGGCAAA
TTCATTGAACATGTGAGCAACGTGACCATTTAA
SEQ ID NO: 11: GLY91 gene; GLY91-At71C1_At71C2_353
ATGGGCAAACAAGAAGATGCCGAACTGGTTATTATTCCGTTTCCGTTTAGCGGTCATATT
CTGGCAACCATTGAACTGGCAAAACGTCTGATTAGCCAGGATAATCCGCGTATTCATACC
ATTACCATTCTGTATTGGGGTCTGCCGTTTATTCCGCAGGCAGATACCATTGCATTTCTG
CGTAGCCTGGTTAAAAATGAACCGCGTATCCGTCTGGTTACCCTGCCGGAAGTTCAGGAT
CCGCCTCCGATGGAACTGTTTGTTGAATTTGCAGAAAGCTATATCCTGGAATATGTGAAA
AAAATGGTGCCGATTATTCGTGAAGCACTGAGCACCCTGCTGAGCAGCCGTGATGAAAGC
GGTAGCGTTCGTGTTGCAGGTCTGGTTCTGGATTTTTTCTGTGTTCCGATGATTGATGTG
GGCAACGAATTTAATCTGCCGAGCTATATCTTTCTGACCTGTAGCGCAGGTTTTCTGGGT
ATGATGAAATATCTGCCGGAACGTCATCGTGAAATCAAAAGCGAATTTAACCGCAGCTTT
AACGAAGAACTGAATCTGATTCCGGGTTATGTTAATAGCGTTCCGACCAAAGTGCTGCCG
AGCGGTCTGTTTATGAAAGAAACCTATGAACCGTGGGTAGAACTGGCCGAACGTTTTCCG
GAAGCAAAAGGTATTCTGGTTAATAGCTATACCGCACTGGAACCGAATGGCTTCAAATAT
TTCGATCGTTGTCCGGATAACTACCCGACCATTTATCCGATTGGTCCGATTCTGTGTAGC
AATGATCGTCCGAATCTGGATCTGAGCGAACGTGATCGTATTCTGAAATGGCTGGATGAT
CAGCCGGAAAGCAGCGTTGTGTTTCTGTGCTTTGGTAGCCTGAAAAGCCTGGCAGCAAGC
CAGATTAAAGAAATTGCACAGGCCCTGGAACTGGTTGGTATTCGTTTTCTGTGGTCAATT
CGTACCGATCCGAAAGAATATGCAAGCCCGAACGAAATCCTGCCGGATGGTTTTATGAAT
CGTGTTATGGGTCTGGGTTTAGTTTGTGGTTGGGCACCGCAGGTTGAAATTCTGGCACAT
AAAGCAATTGGTGGTTTTGTTAGCCATTGCGGTTGGAATAGCATTCTGGAAAGCCTGCGT
TTTGGTGTGCCGATTGCAACCTGGCCGATGTATGCAGAACAGCAGCTGAATGCATTTACC
ATTGTGAAAGAATTAGGTCTGGCACTGGAAATGCGTCTGGATTATGTTAGCGAATATGGC
GAAATTGTCAAAGCCGATGAAATTGCCGGTGCAGTTCGTAGCCTGATGGATGGTGAAGAT
GTTCCGCGTCGTAAACTGAAAGAAATCGCAGAAGCAGGTAAAGAAGCAGTTATGGATGGC
GGTAGCAGCTTTGTTGCAGTTAAACGTTTTATTGATGGCCTGTAA
SEQ ID NO: 13: GLY108 gene; GLY108-Vv92G
ATGGGTAGCCAGCAAGAACATATTGTTATGCTGCCGTTTATGGCACAGGGTCATATTATT
CCGTTTCTGGCACTGGCAAAACAAATTCAGCAGCGTACCGGTTTTACCATTACCATTGCA
AATACACCGCTGAACATTCAGTATCTGCGTACCACCATTAGCACCAGTGATGATAGCAGC
CGTCCGTGTATTCGTCTGGCAGAACTGCCGTTTTGTAGCAGCGATCATGGTCTGCCTCCG
AATACCGAAAATACAGAAGCACTGAGCTTTCATCAGATTGTGGACCTGTTTCATGCAAGC
AAAACCCTGCAGGCACCGTTTCATAGCCTGGTTAGCGGTATTATTGAAAAAGAGGGTCGT
CCGCCTCTGTGCATTATTAGTGATGTTTTTTTTGGTTGGGCAACCGAAGTTGCAAAAAGC
CTGGGCACCAGCAATGTTACCTTTACCACAGGTGGTGGTTATGGCACCGCAGCATATATT
AGCCTGTGGCAAAATCTGCCGCATCGTGCAACCGATAGCGATTATTTTGCACTGCCTGGT
TTTCCGGATAGCTGTCGTTTTCATATTACCCAGCTGCATCAGTACCTGCGTGCAGCAGAT
GGCACCGATGCATGGTCACGTTATTTTCAGCCGCAGATTGCACTGAGCCTGGATAGCAGC
GGTTGGCTGTGTAATACAGCCGAAGAAATTGAACCGCATGGTCTGGAAATTCTGCGCAAT
TATGTTAAACCGCCTGTTTGGACCATTGGTCCGCTGCTGCCTCCGGCACTGCTGAATCAT
AGTCTGAGCAGCGGTAGCAGCATTTTTGGTCAGCGTGCATGGAAAGTTAGCGGTGTTAGT
CCGGAAAAATGTCTGGATTGGCTGGATAAACATCCGCAGAGCAGCGTTCTGTATATTAGC
TTTGGTAGCCAGAATACCATTAGTCCGAGCCAGATGATGGAACTGGCATTAGGTCTGGAA
GATAGCGGTAAACCGTTTATTTGGGTTATTCGTCCTCCGGTGGGTTTTGATATTGAAGGT
GAATTTCGTGCAGAATGGCTGCCGCAGAACTTTGAACAGCGTATGGCAGAAAGCAATCAG
GGTCTGATTGTTCATAAATGGGCTCCGCAGCTGGAAATCCTGAGCCATAAAAGCACCGGT
GTGTTTCTGAGCCATTGTGGTTGGAATAGCGTTATGGAAAGCCTGTGTGTTGGTGTTCCG
ATTATTGGTTGGCCTCTGGCAGCAGAACAGTGTTATAATAGCAAAATGCTGACCGAAGAT
ATGGGTGTTGCAGTTGAACTGACCCGTGGTCGTCAGGGTGCACTGGAACGTAAAGAAGTT
AAACGCGTTATTGAACTGGTGATGGATAGCAAAGGTAAAGGCGAAGAGATGAAAAAAAAG
GCCACCGAAATTGGCGAAAAAATTCGTGATGCAATGCGTGAAGGTGGTAGCAGCCTGAAA
GCAATGGATGATTTTGTTAGCACCATGCTGAGCAAACGTCAGGGTTATTAA
SEQ ID NO: 15: GLY119 gene; GLY119-Pt73Y
ATGGGTAGCCTGGGTCATCAGCTGCATATCTTTTTTCTGCCGTTTTTTGCACATGGCCAT
ATGATTCCGAGCGTTGATATGGCAAAACTGTTTGCAAGCCGTGGTATTAAAACCACCATT
ATTACCACACCGCTGAACGCACCGTTTTTTAGCAAAACCATTCAGAAAACCAAAGAGCTG
GGCTTCGATATTAACATCCTGACCATCAAATTTCCGGCAGCAGAAGCAGGTCTGCCGGAA
GGTTATGAAAATACCGATGCATTTATCTTCAGCGAAAATGCACGTGAGATGACGATCAAA
TTCATTAAAGCAACCACCTTTCTGCAGGCACCGTTTGAAAAAGTTCTGCAAGAATGTCAT
CCGGATTGTATTGTTGCCGATGTTTTTTTTCCGTGGGCAACCGATGCAGCAGCAAAATTT
GGTATTCCGCGTCTGGTTTTTCATGGCACCAGCAATTTTGCACTGAGCGCAAGCGAATGT
GTTCGTCTGTATGAACCGCATAAAAAAGTTAGCAGCGATAGCGAACCGTTTGTTGTTCCG
GATCTGCCTGGTGATATTAAACTGACCAAAAAACAGCTGCCGGATGATGTTCGTGAAAAT
GTGGAAAATGACTTCAGCAAATTCCTGAAAGCAAGCAAAGAAGCAGAACTGCGTAGCTTT
GGTGTTGTTGTGAATAGCTTTTATGAACTGGAACCGGCATATGCGGACTACTACAAAAAA
GTGCTGGGTCGTCGTGCATGGAATGTTGGTCCGGTTAGCCTGTGTAATCGTGATACCGAA
GATAAAGCAGGTCGTGGTAAAGAAACCAGCATTGATCATCATGAATGTCTGAAATGGCTG
GACAGCAAAAAACCGAATAGCGTTGTGTATATTTGCTTTGGTAGCACCACGAATTTTAGC
GATAGCCAGCTGAAAGAAATTGCAGCCGGTCTGGAAGCAAGCGGTCAGCAGTTTATTTGG
GTTGTTCGTCGTAACAAAAAAGGCCAAGAGGATAAAGAAGATTGGCTGCCTGAAGGCTTT
GAAGAACGTATGGAAGGTGTTGGTCTGATTATTCGTGGTTGGGCACCGCAGGTTCTGATT
CTGGATCATGAAGCAATTGGTGCATTTGTTACCCATTGTGGTTGGAATAGCACCCTGGAA
GGTATTACCGCAGGTAAACCGATGGTTACCTGGCCGATTTTTGCAGAACAGTTCTATAAT
GAAAAACTGGTGACCGATGTGCTGAAAACCGGTGTTGGTGTGGGTGTTAAAGAATGGTTT
CGTGTTCATGGTGATCACGTTAAAAGCGAAGCAGTGGAAAAAACCATTACGCAGATTATG
GTTGGTGAAGAGGCCGAAGAAATGCGTAGCCGTGCCAAAAAACTGGGTGAAACCGCACGT
AAAGCAGTTGAAGAAGGTGGTAGCAGCTATAGTGATTTTAATGCCCTGATTGAAGAACTG
CGCTGGCGTCGTCCGTAA
SEQ ID NO: 17: GLY146 gene; GLY146-Zj71G
ATGAAAAAAAGCGAGCTGGTGTTTATTCCGGCACCTGGTATTGGTCATCTGGTTAGCGCA
CTGGAATTTGCAAAACGTCTGATTGATCATGATGATCGCCATAGCATTACCGTTCTGAGC
ATGAAATTTCCGTTTACACCGTTTGCAGATGAGTTTACCAAAAGCCTGGTTGCAAGCCAT
CATGGTATTCAGGTTATTGATCTGCCTCCGGTTGATCCGCCTCCTCCGGAACTGCTGAAT
AGTCCGGAAAATTTTGTTTGCGCCTTTATCGAAAGCCTGATTCCGCATGTTCGTAATACC
CTGACCGATATGCTGAGCAGCAGCAGCCGTGTTAGCGGTCTGGTTCTGGATTTTTTCTGT
CTGAGCCTGATTGATGTTGGTAATGAACTGGGTGTTCCGAGCTATATGTTTCTGACCAGC
AATAGCGGTTTTCTGGGTCTGATGCTGCATATTCCGATTCGTCATAATCAGAAAAGCGCA
GAATTTGAAGAAAGTGATGCCGAAGTTAGCCTGCCTGGTTTTTGGAATCCGGTTCCGGTT
AGCGTTCTGCCTGGTGCAGTTTTTGATAAAGATGGTGGTTATAGCGCCTATCTGAAACTG
GCCAAACGTTTTCGTGATACCAAAGGCATTATTGTGAACACCTTTAGTGAACTGGAACTG
GATAGCGTTAAAAGCCTGAGTGATGGTGAAACCCCTCCGGTTTATATGGTTGGTCCGGTG
ATTGATCTGAAAGGTCAGCCGAATCCGAGCCTGGATCAGGCACAGCGTGAAAAAATCATG
CATTGGCTGGATGATCAGCCGGAAAGCAGCGTTGTTTATCTGTGTTTTGGTAGCATGGGC
ACCTTTGGTGCAATTCAGCTGCGTGAAATTGCAACCGGTCTGGAACGTAGCGGTCATCGT
TTTCTGTGGTCAATGCGTGTTCCGCAGCCTGCAAAAGGTCCGGATGATATTCTGCCGGAA
GGTTTTAGCGAACGTATTGGTGGTCGTGGTCTGATTTGTGGTTGGGCACCGCAGATGGAA
GTTCTGGCACATAAAGCAATTAAAGGCTTTGTTAGCCATTGCGGCTGGAATAGCATTCTG
GAAAGCCTGTGGTTTGGTGTGCCGATTGCAACCTGGCCGATTTATGCAGAACAGCAGATG
AATGCATTTCGCATGGTTAAAGAATTTGGTCTGGCCGTTGAAATGAAACTGGATTATCGT
CTGGGTGATCTGGTTATGGCAGATGAAATTGAAATTGCCGTTCGTCGTCTGATGGATGAT
AGTGATGAACTGCGTAAAAAAGTGAAAGAAATGGGTGAAATGGCACGCCAGGCAATTATT
GAAGGTGGTAGCAGCTATATTGCAGTTGGTCAGCTGATTGAAGATATTCTGGGTAGCATT
AGCTGCGACAACTAA
SEQ ID NO: 19: GLY153 gene; GLY153-Pt72B
ATGGCAGAAACCGATAGTCCGCCTCATGTTGCAATTCTGCCGAGTCCTGGTATGGGTCAT
CTGATTCCGCTGGTTGAACTGGCAAAACGTCTGGTTCATCAGCATAATCTGAGCGTGACC
TTTATTATCCCGACCGATGGTAGCCCGAGCAAAGCACAGCGTAGCGTTCTGGGTAGCCTG
CCGAGCACCATTCATAGCGTTTTTCTGCCTCCGGTTAATCTGAGTGATCTGCCGGAAGAT
GTTAAAATTGAAACCCTGATTAGCCTGACCGTTGCACGTTCACTGCCGAGCCTGCGTGAT
GTTCTGAGCAGCCTGGTTGCAAGCGGCACCCGTGTTGTTGCACTGGTTGTTGACCTGTTT
GGCACCGATGCATTTGATGTTGCACGTGAATTTAAAGCAAGCCCGTATATCTTTTATCCG
GCACCGGCAATGGCACTGAGCCTGTTTTTCTATCTGCCGAAACTGGATGAAATGGTGAGC
TGTGAATATAGCGAAATGCAAGAACCGGTTGAAATTCCGGGTTGTCTGCCGATTCATGGT
GGTGAACTGCTGGATCCGACACGTGATCGTAAAAATGATGCATATAAATGGCTGCTGCAT
CACAGCAAACGTTATCGTCTGGCCGAAGGTGTTATGGTGAATAGCTTTATTGATCTGGAA
CGTGGTGCACTGAAAGCACTGCAAGAAGTTGAACCGGGTAAACCGCCTGTTTATCCGGTT
GGTCCGCTGGTGAATATGGATAGCAATACCAGCGGTGTTGAAGGTAGCGAATGTCTGAAA
TGGCTGGATGATCAGCCGCTGGGTAGCGTGCTGTTTGTTAGCTTTGGTAGCGGTGGCACC
CTGAGCTTTGATCAGATTACCGAACTGGCACTGGGTTTAGAAATGAGCGAACAGCGTTTT
CTGTGGGTTGCCCGTGTTCCGAATGATAAAGTTGCAAATGCAACCTATTTCAGCGTGGAT
AATCACAAAGATCCGTTTGATTTTCTGCCGAAGGGTTTTCTGGATCGTACCAAAGGTCGT
GGTCTGGTTGTTCCGAGCTGGGCACCGCAGGCACAGGTTCTGAGCCATGGTAGCACCGGT
GGTTTTCTGACCCATTGTGGTTGGAATAGCACCCTGGAAAGCGTTGTTAATGCAGTTCCG
CTGATTGTTTGGCCTCTGTATGCAGAACAGAAAATGAATGCATGGATGCTGACCAAAGAT
GTTGAAGTTGCACTGCGTCCGAAAGCAAGCGAAAATGGTCTGATTGGTCGTGAAGAAATT
GCCAATATTGTGCGTGGTCTGATGGAAGGTGAAGAAGGTAAACGCGTTCGTAATCGTATG
AAAGATCTGAAAGATGCAGCCGCAGAAGTTCTGAGCGAAGCAGGTAGCAGCACCAAAGCA
CTGAGTGAAGTTGCCCGTAAATGGAAAAACCATAAATGTACCCAGGACTGCAACTAA
SEQ ID NO: 21: GLY163 gene; GLY163-Cp71B
ATGACCGGTGAACTGATTTTTATCCCGATGCCGAGCCTGAGCCATATTGCAAGCACCATG
GAAATTGCAAAACTGCTGGTTCATCGTGATGATCGTCTGAGCATTACCGTTCTGCTGATT
AGCAGCCAGTATACCACCTCAATTACCACCTATATTAACAGCCTGATTGCCAGCAGCGAT
TATGATCGTATTCGTTTTATTCATCTGCCGGAACTGGATAGCGAAGAAGAACCGAAACGT
CCGTTTATGAGCGTGATTGATGATAACAAACCGATCGTTAAAGAAGCCGTTACCAATCTG
GCACTGAGCTTTGATCCGAGCCATCGTCTGGCAGGTTTTGTTATTGATATGTTTTGCGTG
GGCATGATTGAAGTTGCAGATGAACTGGGTCTGCCGAGCTATCCGTTTTTTACCAGCAGC
ACCAGCTTTCTGGCCCTGCAGTTTCATGTTCAGACCCTGGCCGATGAAGAAGAAGTTGAT
ATTACCGAGTTTAAGAACTCCGATGTTATGCTGCCGATTCCTGGTCTGGTTAATCCGCTG
CCTGCAAAAACCATTCTGCCGAGTGCAATGCTGAATAAAGATTGGCTGCCGTATGTTCTG
AATGGTGCACGTGGTTTTCGTAAAACGAAAGGCATTATGGTTAACAGCTTTGCCGAAATT
GAAAGCAATGCAGTTACCAGCCTGAGCAATAGCACCGTTCCGCCTGTTTATACCGTTGGT
CCGATTATTAACTTTAAAGGTGATGGTCAGGATAGCGATACCTGTACCGCACACAAATAT
AGCAATATTATGACCTGGCTGGATGATCAGCCTCCGAGCAGCGTTCTGTTTCTGTGTTTT
GGTAGCCTGGGTAGCTTTGATGAAGAACAGGTTAAAGAAATTGCACGTGCCCTGGAAGGT
AGCGGTCATCGTTTTCTGTGGTCACTGCGTCGTCCGCCTCCGAAAGATAAAACCATGAGC
TTTCCGACCGAATATGAAAACTTTGAAGAAGTGCTGCCGGAAGGTTTTGTGGATCGCACC
GTTGGTATGGGTAAAGTTATGGGTTGGGCACCGCAGGTTGCAGTTCTGGCACATCCGAGC
ATTGGTGGTTTTGTGACCCATTGTGGTTGGAATAGCATTCTGGAAAGCGTTTGGTTTGGT
GTTCCGATGGCAGCATGGCCTCTGTATGCAGAACAGCAGTTTAATGCATTTCATATGGTG
GTGGAACTGGGTTTAGCAGTGGAAATCAAAATGGATTATCGCAAAGATTATGCCATTCTG
GGCCTGCAAGAAGAACGCGTTAGCGCAGAAGTTATTGAAAAAGGTATTCGTTGTCTGATG
GAAGAGGATAATGATGCCCGTAAAAAAGTGAAAGAAATGAGCGAAATTAGCCGCAAAGCA
CTGATGGATGGTGGTAGCAGCCATGCCGTTCTGGGTCAGTTTATTGAAGATGTGATGAAT
AACATCAGCGCCTAA
SEQ ID NO: 23: GLY164 gene; GLY164-Cs71M
ATGAAAGAAAAAGCCGAACTGGTGTTTATTCCGAGTCCGAGCGTTGGTCATCTGGTTAGC
GTTATTGTTTTTGCCAAACGTCTGCTGCTGCTGGATCGTGATAATGCAGCAGGTAGCAAT
AATTCCTTTAGCATTACCGTTCTGGTGATGCAAGAACATACCGCAACCGCAAGCGATAAT
GATGCACATATCAAAAGCCTGGCTGGTGCCGGTGCAACCGCAGATGTTAGCATTCGTTTT
ATTGGTGTGCCGAAAATGAATCCGCCTCCGCTGGATTATTTCAAAAGTCCGGAAAAATTC
ATCACCGAGTATGTGGATAGCCACAAAGATTGTATTAAAGAGGCCATTATTGAACACGTG
CTGAACAACAATGTTAAAATTGCAGGTCTGGTGCTGGATTTTTTCTGTAGCAGCATGATT
GATACCGCAAATGAACTGGGTGTTCCGAGCTATGTGTTTTTTACCAGCGGTGCAGCATTT
CTGGGTTTTGTTCTGCATCTGCCGACACGTGGTGGTGAAGAATTTGAAGAAAGCGAAGCA
GATAGCGCAGATATCTTTACCTATGCAAATCCGGTTCCGTATCGTGTTCTGCCGAGCCTG
TGTTTTAACAAACATGGTGGTTTTAGCGCCTTTGAAAATTTTGGTCGTCGCTTCAAAGAA
ACCAAAGGCATTATTGTGAACACCTTCGAAGAACTGGAAAGCCATGCAGTTGAATACCTG
ATGAAATGTGATGGTGTTCCGCCTGTTTATAATGTTGGTCCGATGATTGATCTGCATGGT
GATATTCATGCACGTCCGCGTGGCGGTGGCACCCAGCGTGATGAAATTATTCGTTGGCTG
GATGATCAGCCTGCAAGCAGCGTTGTTTTTCTGTGTTTTGGTAGCATGGGTAGCTTTGGC
GAAGAACAAATTAAAGAAGTTGCAAGCGGTCTGGGTAAAAGCGGTGTTCGTTTTCTGTGG
TCACTGCGTAAAACCCCTCCGAAAGATCGTCCGGAAATGCCTGGTGAATATACCTGTGTT
GAAGATATTCTGCCTCGCGGTTTTCAAGAACGTAGCAAAGGTCGTGGTATGATTTGTGGT
TGGGCACCGCAGAAAGAAGTTCTGGCACATAGCGCAATTGGTGGCTTTGTTAGCCATTGC
GGTTGGAATAGCATTCTGGAAAGCGTTTGGTTTGGCGTTCCGATTGTTACCTGGCCGATT
TATGCCGAACAGCAGATTAATGCATTTCAGATGGTTCGTGATCTGGGTTTAGCAGTTGAA
CTGCGTCTGGATTATCGTCGTGGCACCGAAAATCATGTTATGGCAGATGAACTGGCACGT
GCGGTTGGTTGTGTTATGGATGGTGATAGCGAAGTTCGTAAACGTGTGAAAGAAGTGAGC
GAAAAAGCACGTCTGGCACTGCGTGATGGTGGTAGCAGCTATGCAGCAACCGGTCGTCTG
ATTGAAGATTTATTTGGTAGCGTGAGCTAA
SEQ ID NO: 25: GLY169 gene; GLY169-Eg71D
ATGAAAAAGCCGGAACTGATCTTTATTCCGGCACCTGGTGTTGGTCATGTTATTAGCGCA
CTGGAATTTGCAGCACGTCTGCTGGGTCATGATCCGCGTATTAGCATTACCGTTCTGGCA
ATTAAAGCACCGTTTAGCAGCCATCTGGATGCATATACCAAAAGCCTGGTTGCAAGCCAG
CCTCGTATTGCACTGATTGATCTGCCTCCGGTTGATCCGCCTCCTCCGGAACTGCTGCGT
AGCGTTGAACATTATGTTACCGTTTATATCGAGAGCTATATCCCGCATGTTCAGCGTGCA
GTTGGTGAAATTCAGAGCAGCCGTAGCGATAGCGGTAGCGATCGTAGCGTGGCAGGTTTT
GTTCTGGATTTTTTCTGTACCAGCATGATTGATGTTGCCGATGGTTTTAGCCTGCCGAGT
TATATCTATCTGACCAGCAATGCACATTTCCTGGATCTGATGTTTCATCTGCCTGCACGT
CATGATCAGATTGGTCGTGAAGCCCGTCTGAGCGATCCTGATCTGCCGCTGCCGAATTTT
GCAAATCCGGTTCCGGTTAGCGTTCTGCCGAGCGCAGTTCTGGATGGTGAAGCCGGTGGT
TATGCAGCATATGTTAAACTGGCACGTCGTTTTCGTGATGTTCGTGGTATTATGGTGAAC
ACCTTTATGGAACTGGAACGTAGTGCACTGGGTAGCTTTAGCGGTGGTAGCTGGGATGGT
CCGGCAGTTTATGCCGTTGGTCCGGTTATTGATCCGAAAGGTGTGCCGGATCCGAGCCTG
GATCGTGCACATTGGGAAAAAATTCGTGAATGGCTGGATGAACAGCCTCCGAGCAGCGTT
GTTTTTCTGTGTTTTGGTAGCCTGGGTAGTTTTGATGCCGATCAGGTTAAAGAAATTGCC
AGCGGTATTGAACGTAGCGGTCATAAATTTCTGTGGTCACTGCGTCCGGAAAGCAGCCAG
AGCCAGCTGCCGGTTGGTTTTCTGGAACGGATGCGTGGTCGCGGTATTATTTGTGGTTGG
ACACCGCAGGTTGAAGTTCTGGGCCATGCAGCAATTGGTGGTTTTGTTAGCCATTGCGGT
TGGAATAGCATTCTGGAAAGCCTGTGGTATGGTGTTCCGATTGTTACCTGGCCGATTTAT
GCAGAACAGCAGCTGAATGCATTTACCATGGTGAAAGAACTGGGTTTAGCAGTTGAACTG
CGTCTGGATTACAAAGGTCAAGGTATTGGTGGTCATCTGGTTCCGGCAGAAGAAATTGAA
AAAGCAGTTGGTCGTCTGATGGGAGATGATGCAGCAGAAACCCGTAAAAAAGTGCGTGAA
ATGCGTGAAGCAGCACGTAATGCAGTTAAAGAAGGTGGTAGCAGCTACGATAGCATCGGT
CGCCTGATTCAGGATTTTATCAACAGCATCGAATAA
SEQ ID NO: 27: GLY170 gene; GLY170-Zj71M
ATGAAGAAAATCAACCTGGTGGTTATTCCGTTTGCCGGTGTTGGTCATCTGGTTAGCACC
ATTGAATTCATTAAACGTCTGCTGAGCCGTGATGATCGTTTTAGCGTTACCATTCTGGTG
ATTAAAAGTCCGCTGGAACATAGCCTGGATCCGAATACCAGTCCGCCTAGCCTGCCGAGC
AGCCATCCGCATATTCAGTTTATTAACGTTCGTAGCCTGAATCCGCCTCCGATTGAAGTT
TTTCTGAAAAGTCCGGAAAAATACTTTACCCTGTACATCGAGAGCTACAAGCTGAAAGTG
AAAGAAATCATTGTGGATCATGTTCTGCCGAATAGCGCACCGTTAGGTGGTCTGTTTCTG
GATATGTTTAGCACCAGCATGGTTGATGTTGCACAAGAACTGGGTGTTCCGAGCTATGTG
TTTCTGACCAGCGGTGCAGGTTTTCTGGGTTTTGTTCTGTATCTGCAGACCCGTCATGAT
CGCGGTGGTGCAGAATTTGAACTGAGCGATATTGAAAGCTATATCCCGAGTTATGTTAAT
CCGGTTCCGAGCCGTGTGCTGCCGAGCCTGGCATTTAATCGTGAAGGTGGTTATGTTAGC
TTTGTGAATGTTGCCCGTAATATGAAAAAAACCAAAGGCATTATTGTGAACACCGTGTTT
GAACTGGAAAGCCATGCAATTAGCAGCCTGCTGATGGATGGTGCAACCCCTCCGGTTTAT
GCAGTTGGTCCGCTGATTGATCATAAAGGTGAAAGCCATTTTCGTACCGATCAGGCAAAA
CGCGATAAAATCATTAAATGGCTGGATGATCAGCCGACCAAAAGCGTTATTTTTCTGTGC
TTTGGTTCCTTTGGTAGCTTTGGTGTTCCTCAGCTGCGTGAAATTGCCATTGGTCTGGAA
CAGAGCGGTGCCCGTTTCCTGTGGTCAATTCGTCAGGCCAAAAGCATTTTTGAAATGCCG
ATTGAATGCGAGAAACCGGAAGAATTTCTGCCGCAGGGCTTTCTGGATCGTACCAAAGAA
TTAGGTATGATTTGTGGTTGGACACCGCAGATTGAAGTGCTGGCACATCGTGCAATTGGT
GGTTTTGTGAGCCATTGCGGTTGGAATAGCATTCTGGAAAGTCTGTGGCATGGTGTGCCG
ATTGTTACCTGGCCTCTGTATGCAGAACAGCAGATTAATGCATTTCAGATCGTGAAAGAT
TTAGGCCTGGGTGTTGAACTGCGTCTGGATTATCGTCGTGATGGCGGTGATGTTGATGTG
GTTATGCGTGATGAAATTGAAAAAGCCGTTCGCTATGTTATGGATGGCGATAGCGAAGTT
CGTCAGCGTGTTCAAGAGGCAAGCGAAAAATGTCGTAAAGCAGTTGTGGAAGGTGGTAGC
AGCTTTAGCAGCATTGGTAGCCTGATTGAAGCAATTCTGGCCAACATTAGCTAA

In some embodiments, the glycosyltransferase has an amino acid sequence that is at least 70% identical to any one of SEQ ID NOs: 2, 4, 6, 8, 10, 12, 14, 16, 18, 20, 22, 24, and 26.

In some embodiments, the glycosyltransferase has an amino acid sequence that is at least 75% identical to any one of SEQ ID NOs: 2, 4, 6, 8, 10, 12, 14, 16, 18, 20, 22, 24, and 26.

In some embodiments, the glycosyltransferase has an amino acid sequence that is at least 80% identical to any one of SEQ ID NOs: 2, 4, 6, 8, 10, 12, 14, 16, 18, 20, 22, 24, and 26.

In some embodiments, the glycosyltransferase has an amino acid sequence that is at least 85% identical to any one of SEQ ID NOs: 2, 4, 6, 8, 10, 12, 14, 16, 18, 20, 22, 24, and 26.

In some embodiments, the glycosyltransferase has an amino acid sequence that is at least 90% identical to any one of SEQ ID NOs: 2, 4, 6, 8, 10, 12, 14, 16, 18, 20, 22, 24, and 26.

In some embodiments, the glycosyltransferase has an amino acid sequence that is at least 95% identical to any one of SEQ ID NOs: 2, 4, 6, 8, 10, 12, 14, 16, 18, 20, 22, 24, and 26.

In some embodiments, the glycosyltransferase has an amino acid sequence that is at least 96% identical to any one of SEQ ID NOs: 2, 4, 6, 8, 10, 12, 14, 16, 18, 20, 22, 24, and 26.

In some embodiments, the glycosyltransferase has an amino acid sequence that is at least 97% identical to any one of SEQ ID NOs: 2, 4, 6, 8, 10, 12, 14, 16, 18, 20, 22, 24, and 26.

In some embodiments, the glycosyltransferase has an amino acid sequence that is at least 98% identical to any one of SEQ ID NOs: 2, 4, 6, 8, 10, 12, 14, 16, 18, 20, 22, 24, and 26.

In some embodiments, the glycosyltransferase has an amino acid sequence that is at least 99% identical to any one of SEQ ID NOs: 2, 4, 6, 8, 10, 12, 14, 16, 18, 20, 22, 24, and 26.

In some embodiments, the glycosyltransferase has an amino acid sequence that is at least 100% identical to any one of SEQ ID NOs: 2, 4, 6, 8, 10, 12, 14, 16, 18, 20, 22, 24, and 26.

In some embodiments, the glycosyltransferase has an amino acid sequence any one of SEQ ID NOs: 2, 4, 6, 8, 10, 12, 14, 16, 18, 20, 22, 24, and 26.

In some embodiments, the glycosyltransferase includes an amino acid sequence that is at least 70% identical to any one of SEQ ID NOs: 2, 4, 6, 8, 10, 12, 14, 16, 18, 20, 22, 24, and 26.

In some embodiments, the glycosyltransferase includes an amino acid sequence that is at least 75% identical to any one of SEQ ID NOs: 2, 4, 6, 8, 10, 12, 14, 16, 18, 20, 22, 24, and 26.

In some embodiments, the glycosyltransferase includes an amino acid sequence that is at least 80% identical to any one of SEQ ID NOs: 2, 4, 6, 8, 10, 12, 14, 16, 18, 20, 22, 24, and 26.

In some embodiments, the glycosyltransferase includes an amino acid sequence that is at least 85% identical to any one of SEQ ID NOs: 2, 4, 6, 8, 10, 12, 14, 16, 18, 20, 22, 24, and 26.

In some embodiments, the glycosyltransferase includes an amino acid sequence that is at least 90% identical to any one of SEQ ID NOs: 2, 4, 6, 8, 10, 12, 14, 16, 18, 20, 22, 24, and 26.

In some embodiments, the glycosyltransferase includes an amino acid sequence that is at least 95% identical to any one of SEQ ID NOs: 2, 4, 6, 8, 10, 12, 14, 16, 18, 20, 22, 24, and 26.

In some embodiments, the glycosyltransferase includes an amino acid sequence that is at least 96% identical to any one of SEQ ID NOs: 2, 4, 6, 8, 10, 12, 14, 16, 18, 20, 22, 24, and 26.

In some embodiments, the glycosyltransferase includes an amino acid sequence that is at least 97% identical to any one of SEQ ID NOs: 2, 4, 6, 8, 10, 12, 14, 16, 18, 20, 22, 24, and 26.

In some embodiments, the glycosyltransferase includes an amino acid sequence that is at least 98% identical to any one of SEQ ID NOs: 2, 4, 6, 8, 10, 12, 14, 16, 18, 20, 22, 24, and 26.

In some embodiments, the glycosyltransferase includes an amino acid sequence that is at least 99% identical to any one of SEQ ID NOs: 2, 4, 6, 8, 10, 12, 14, 16, 18, 20, 22, 24, and 26.

In some embodiments, the glycosyltransferase includes an amino acid sequence that is at least 100% identical to any one of SEQ ID NOs: 2, 4, 6, 8, 10, 12, 14, 16, 18, 20, 22, 24, and 26.

In some embodiments, the glycosyltransferase includes an amino acid sequence any one of SEQ ID NOs: 2, 4, 6, 8, 10, 12, 14, 16, 18, 20, 22, 24, and 26.

In some embodiments, the glycosyltransferase is GLY70, GLY87, GLY77, GLY169, GLY163, GLY74, GLY170, GLY91, GLY164, GLY108, GLY146, GLY119, GLY73, or GLY153.

In some embodiments, the glycosyltransferase is GLY70. In some embodiments, the glycosyltransferase is GLY87. In some embodiments, the glycosyltransferase is GLY77. In some embodiments, the glycosyltransferase is GLY169. In some embodiments, the glycosyltransferase is GLY163. In some embodiments, the glycosyltransferase is GLY74. In some embodiments, the glycosyltransferase is GLY170. In some embodiments, the glycosyltransferase is GLY91. In some embodiments, the glycosyltransferase is GLY164. In some embodiments, the glycosyltransferase is GLY108. In some embodiments, the glycosyltransferase is GLY146. In some embodiments, the glycosyltransferase is GLY119. In some embodiments, the glycosyltransferase is GLY73. In some embodiments, the glycosyltransferase is GLY153.

SEQ ID NO: 2: GLY70 protein; GLY70-At71C2
MAKQQEAELIFIPFPIPGHILATIELAKRLISHQPSRIHTITILHWSLPFLPQSDTIAFL
KSLIETESRIRLITLPDVQNPPPMELFVKASESYILEYVKKMVPLVRNALSTLLSSRDES
DSVHVAGLVLDFFCVPLIDVGNEFNLPSYIFLTCSASFLGMMKYLLERNRETKPELNRSS
DEETISVPGFVNSVPVKVLPPGLFTTESYEAWVEMAERFPEAKGILVNSFESLERNAFDY
FDRRPDNYPPVYPIGPILCSNDRPNLDLSERDRILKWLDDQPESSVVFLCFGSLKSLAAS
QIKEIAQALELVGIRFLWSIRTDPKEYASPNEILPDGFMNRVMGLGLVCGWAPQVEILAH
KAIGGFVSHCGWNSILESLRFGVPIATWPMYAEQQLNAFTIVKELGLALEMRLDYVSEYG
EIVKADEIAGAVRSLMDGEDVPRRKLKEIAEAGKEAVMDGGSSFVAVKRFIDGL*
SEQ ID NO: 4: GLY73 protein; GLY73-Qs88D_2
MEDAIVLYPSPGRGHLISMVELGKLILKNHPSESITILILSEPNTNANSTAPNYVASSTT
EYITTVNSTTPSITFHHLPPISEVPPSTSSFVELSYLIPRFNNONLHQILKTISQTSKLK
AFIIDFFCDAAFEVATNLDIPTYYFFTSGACYLAALLYRPTLHKKVDKSLKDLGNTLLDI
PGLPPIQASDLPEPWKDRTSKEYQYSLNVAAHMAKSNGLLVNTFDLLETKAIKAISGGLC
VPDRTTPPIFCIGPLISDSNQDVEDHECLNWLNSQPSRSVVFLCFGSLGLFSAKQLKEIA
VGLENSGQRFLWVLRNPPSDNDKDPNLDELLPKGFLERTKEKGFVVKQWAPQVAVLRHDS
VGGFVTHCGWNSVLEAVWCRVPMVGWPLYAEQRLNSVVLVEEIKLASGLNESEDGLVSAA
ELEKRVKELMDSEVGTEVRERVSGLRDEAVTAVKEGGSSHVALAKLAELWRQN*
SEQ ID NO: 6: GLY74 protein; GLY74-At71D1
MRNVELIFIPTPTVGHLVPFLEFARRLIEQDDRIRITILLMKLQGQSHLDTYVKSIASSQ
PFVRFIDVPELEEKPTLGSTQSVEAYVYDVIERNIPLVRNIVMDILTSLALDGVKVKGLV
VDFFCLPMIDVAKDISLPFYVFLTINSGFLAMMQYLADRHSRDTSVFVRNSEEMLSIPGF
VNPVPANVLPSALFVEDGYDAYVKLAILFTKANGILVNSSFDIEPYSVNHFLQEQNYPSV
YAVGPIFDLKAQPHPEQDLTRRDELMKWLDDQPEASVVELCFGSMARLRGSLVKEIAHGL
ELCQYRFLWSLRKEEVTKDDLPEGELDRVDGRGMICGWSPQVEILAHKAVGGFVSHCGWN
SIVESLWFGVPIVTWPMYAEQQLNAFLMVKELKLAVELKLDYRVHSDEIVNANEIETAIR
YVMDTDNNVVRKRVMDISQMIQRATKNGGSSFAAIEKFIYDVIGIKP*
SEQ ID NO: 8: GLY77 protein; GLY77-Sr71E1
MSTSELVFIPSPGAGHLPPTVELAKLLLHRDQRLSVTIIVMNLWLGPKHNTEARPCVPSL
RFVDIPCDESTMALISPNTFISAFVEHHKPRVRDIVRGIIESDSVRLAGFVLDMFCMPMS
DVANEFGVPSYNYFTSGAATLGLMEHLQWKRDHEGYDATELKNSDTELSVPSYVNPVPAK
VLPEVVLDKEGGSKMFLDLAERIRESKGIIVNSCQAIERHALEYLSSNNNGIPPVFPVGP
ILNLENKKDDAKTDEIMRWLNEQPESSVVFLCFGSMGSFNEKQVKEIAVAIERSGHRFLW
SLRRPTPKEKIEFPKEYENLEEVLPEGELKRTSSIGKVIGWAPQMAVLSHPSVGGFVSHC
GWNSTLESMWCGVPMAAWPLYAEQTLNAFLLVVELGLAAEIRMDYRTDTKAGYDGGMEVT
VEEIEDGIRKLMSDGEIRNKVKDVKEKSRAAVVEGGSSYASIGKFIEHVSNVTI*
SEQ ID NO: 10: GLY87 protein; GLY87-At71C1_Sr71E1_354
MGKQEDAELVIIPFPFSGHILATIELAKRLISQDNPRIHTITILYWGLPFIPQADTIAFL
RSLVKNEPRIRLVTLPEVQDPPPMELFVEFAESYILEYVKKMVPIIREALSTLLSSRDES
GSVRVAGLVLDFFCVPMIDVGNEFNLPSYIFLTCSAGFLGMMKYLPERHREIKSEENRSE
NEELNLIPGYVNSVPTKVLPSGLFMKETYEPWVELAERFPEAKGILVNSYTALEPNGFKY
FDRCPDNYPTIYPIGPILNLENKKDDAKTDEIMRWLNEQPESSVVFLCFGSMGSFNEKQV
KEIAVAIERSGHRFLWSLRRPTPKEKIEFPKEYENLEEVLPEGFLKRTSSIGKVIGWAPQ
MAVLSHPSVGGFVSHCGWNSTLESMWCGVPMAAWPLYAEQTLNAFLLVVELGLAAEIRMD
YRTDTKAGYDGGMEVTVEEIEDGIRKLMSDGEIRNKVKDVKEKSRAAVVEGGSSYASIGK
FIEHVSNVTI*
SEQ ID NO: 12: GLY91 protein; GLY91-At71C1_At71C2_353
MGKQEDAELVIIPFPFSGHILATIELAKRLISQDNPRIHTITILYWGLPFIPQADTIAFL
RSLVKNEPRIRLVTLPEVQDPPPMELFVEFAESYILEYVKKMVPIIREALSTLLSSRDES
GSVRVAGLVLDFFCVPMIDVGNEFNLPSYIFLTCSAGFLGMMKYLPERHREIKSEFNRSF
NEELNLIPGYVNSVPTKVLPSGLFMKETYEPWVELAERFPEAKGILVNSYTALEPNGFKY
FDRCPDNYPTIYPIGPILCSNDRPNLDLSERDRILKWLDDQPESSVVFLCFGSLKSLAAS
QIKEIAQALELVGIRFLWSIRTDPKEYASPNEILPDGFMNRVMGLGLVCGWAPQVEILAH
KAIGGFVSHCGWNSILESLRFGVPIATWPMYAEQQLNAFTIVKELGLALEMRLDYVSEYG
EIVKADEIAGAVRSLMDGEDVPRRKLKEIAEAGKEAVMDGGSSFVAVKRFIDGL*
SEQ ID NO: 14: GLY108 protein; GLY108-Vv92G
MGSQQEHIVMLPFMAQGHIIPFLALAKQIQQRTGFTITIANTPLNIQYLRTTISTSDDSS
RPCIRLAELPFCSSDHGLPPNTENTEALSFHQIVDLFHASKTLQAPFHSLVSGIIEKEGR
PPLCIISDVFFGWATEVAKSLGTSNVTFTTGGGYGTAAYISLWQNLPHRATDSDYFALPG
FPDSCRFHITQLHQYLRAADGTDAWSRYFQPQIALSLDSSGWLCNTAEEIEPHGLEILRN
YVKPPVWTIGPLLPPALLNHSLSSGSSIFGQRAWKVSGVSPEKCLDWLDKHPQSSVLYIS
FGSQNTISPSQMMELALGLEDSGKPFIWVIRPPVGFDIEGEFRAEWLPQNFEQRMAESNQ
GLIVHKWAPQLEILSHKSTGVFLSHCGWNSVMESLCVGVPIIGWPLAAEQCYNSKMLTED
MGVAVELTRGRQGALERKEVKRVIELVMDSKGKGEEMKKKATEIGEKIRDAMREGGSSLK
AMDDFVSTMLSKRQGY*
SEQ ID NO: 16: GLY119 protein; GLY119-Pt73Y
MGSLGHQLHIFFLPFFAHGHMIPSVDMAKLFASRGIKTTIITTPLNAPFFSKTIQKTKEL
GFDINILTIKFPAAEAGLPEGYENTDAFIFSENAREMTIKFIKATTFLQAPFEKVLQECH
PDCIVADVFFPWATDAAAKFGIPRLVFHGTSNFALSASECVRLYEPHKKVSSDSEPFVVP
DLPGDIKLTKKQLPDDVRENVENDFSKFLKASKEAELRSFGVVVNSFYELEPAYADYYKK
VLGRRAWNVGPVSLCNRDTEDKAGRGKETSIDHHECLKWLDSKKPNSVVYICFGSTTNFS
DSQLKEIAAGLEASGQQFIWVVRRNKKGQEDKEDWLPEGFEERMEGVGLIIRGWAPQVLI
LDHEAIGAFVTHCGWNSTLEGITAGKPMVTWPIFAEQFYNEKLVTDVLKTGVGVGVKEWF
RVHGDHVKSEAVEKTITQIMVGEEAEEMRSRAKKLGETARKAVEEGGSSYSDFNALIEEL
RWRRP*
SEQ ID NO: 18: GLY146 protein; GLY146-Zj71G
MKKSELVFIPAPGIGHLVSALEFAKRLIDHDDRHSITVLSMKFPFTPFADEFTKSLVASH
HGIQVIDLPPVDPPPPELLNSPENFVCAFIESLIPHVRNTLTDMLSSSSRVSGLVLDFFC
LSLIDVGNELGVPSYMFLTSNSGFLGLMLHIPIRHNQKSAEFEESDAEVSLPGFWNPVPV
SVLPGAVFDKDGGYSAYLKLAKRFRDTKGIIVNTFSELELDSVKSLSDGETPPVYMVGPV
IDLKGQPNPSLDQAQREKIMHWLDDQPESSVVYLCFGSMGTFGAIQLREIATGLERSGHR
FLWSMRVPQPAKGPDDILPEGESERIGGRGLICGWAPQMEVLAHKAIKGFVSHCGWNSIL
ESLWFGVPIATWPIYAEQQMNAFRMVKEFGLAVEMKLDYRLGDLVMADEIEIAVRRLMDD
SDELRKKVKEMGEMARQAIIEGGSSYIAVGQLIEDILGSISCDN*
SEQ ID NO: 20: GLY153 protein; GLY153-Pt72B
MAETDSPPHVAILPSPGMGHLIPLVELAKRLVHQHNLSVTFIIPTDGSPSKAQRSVLGSL
PSTIHSVFLPPVNLSDLPEDVKIETLISLTVARSLPSLRDVLSSLVASGTRVVALVVDLF
GTDAFDVAREFKASPYIFYPAPAMALSLFFYLPKLDEMVSCEYSEMQEPVEIPGCLPIHG
GELLDPTRDRKNDAYKWLLHHSKRYRLAEGVMVNSFIDLERGALKALQEVEPGKPPVYPV
GPLVNMDSNTSGVEGSECLKWLDDQPLGSVLFVSFGSGGTLSFDQITELALGLEMSEQRF
LWVARVPNDKVANATYFSVDNHKDPFDFLPKGFLDRTKGRGLVVPSWAPQAQVLSHGSTG
GFLTHCGWNSTLESVVNAVPLIVWPLYAEQKMNAWMLTKDVEVALRPKASENGLIGREEI
ANIVRGLMEGEEGKRVRNRMKDLKDAAAEVLSEAGSSTKALSEVARKWKNHKCTQDCN*
SEQ ID NO: 22: GLY163 protein; GLY163-Cp71B
MTGELIFIPMPSLSHIASTMEIAKLLVHRDDRLSITVLLISSQYTTSITTYINSLIASSD
YDRIRFIHLPELDSEEEPKRPFMSVIDDNKPIVKEAVTNLALSFDPSHRLAGFVIDMFCV
GMIEVADELGLPSYPFFTSSTSFLALQFHVQTLADEEEVDITEFKNSDVMLPIPGLVNPL
PAKTILPSAMLNKDWLPYVLNGARGFRKTKGIMVNSFAEIESNAVTSLSNSTVPPVYTVG
PIINFKGDGQDSDTCTAHKYSNIMTWLDDQPPSSVLFLCFGSLGSEDEEQVKEIARALEG
SGHRFLWSLRRPPPKDKTMSFPTEYENFEEVLPEGFVDRTVGMGKVMGWAPQVAVLAHPS
IGGFVTHCGWNSILESVWFGVPMAAWPLYAEQQFNAFHMVVELGLAVEIKMDYRKDYAIL
GLQEERVSAEVIEKGIRCLMEEDNDARKKVKEMSEISRKALMDGGSSHAVLGQFIEDVMN
NISA*
SEQ ID NO: 24: GLY164 protein; GLY164-Cs71M
MKEKAELVFIPSPSVGHLVSVIVFAKRLLLLDRDNAAGSNNSFSITVLVMQEHTATASDN
DAHIKSLAGAGATADVSIRFIGVPKMNPPPLDYFKSPEKFITEYVDSHKDCIKEAIIEHV
LNNNVKIAGLVLDFFCSSMIDTANELGVPSYVFFTSGAAFLGFVLHLPTRGGEEFEESEA
DSADIFTYANPVPYRVLPSLCFNKHGGFSAFENFGRRFKETKGIIVNTFEELESHAVEYL
MKCDGVPPVYNVGPMIDLHGDIHARPRGGGTQRDEIIRWLDDQPASSVVFLCFGSMGSFG
EEQIKEVASGLGKSGVRFLWSLRKTPPKDRPEMPGEYTCVEDILPRGFQERSKGRGMICG
WAPQKEVLAHSAIGGFVSHCGWNSILESVWFGVPIVTWPIYAEQQINAFQMVRDLGLAVE
LRLDYRRGTENHVMADELARAVGCVMDGDSEVRKRVKEVSEKARLALRDGGSSYAATGRL
IEDLFGSVS*
SEQ ID NO: 26: GLY169 protein; GLY169-Eg71D
MKKPELIFIPAPGVGHVISALEFAARLLGHDPRISITVLAIKAPFSSHLDAYTKSLVASQ
PRIALIDLPPVDPPPPELLRSVEHYVTVYIESYIPHVORAVGEIQSSRSDSGSDRSVAGF
VLDFFCTSMIDVADGFSLPSYIYLTSNAHFLDLMFHLPARHDQIGREARLSDPDLPLPNF
ANPVPVSVLPSAVLDGEAGGYAAYVKLARRFRDVRGIMVNTFMELERSALGSFSGGSWDG
PAVYAVGPVIDPKGVPDPSLDRAHWEKIREWLDEQPPSSVVFLCFGSLGSFDADQVKEIA
SGIERSGHKFLWSLRPESSQSQLPVGFLERMRGRGIICGWTPQVEVLGHAAIGGFVSHCG
WNSILESLWYGVPIVTWPIYAEQQLNAFTMVKELGLAVELRLDYKGQGIGGHLVPAEEIE
KAVGRLMGDDAAETRKKVREMREAARNAVKEGGSSYDSIGRLIQDFINSIE*
SEQ ID NO: 28: GLY170 protein; GLY170-Zj71M
MKKINLVVIPFAGVGHLVSTIEFIKRLLSRDDRFSVTILVIKSPLEHSLDPNTSPPSLPS
SHPHIQFINVRSLNPPPIEVFLKSPEKYFTLYIESYKLKVKEIIVDHVLPNSAPLGGLFL
DMFSTSMVDVAQELGVPSYVFLTSGAGFLGFVLYLQTRHDRGGAEFELSDIESYIPSYVN
PVPSRVLPSLAFNREGGYVSFVNVARNMKKTKGIIVNTVFELESHAISSLLMDGATPPVY
AVGPLIDHKGESHFRTDQAKRDKIIKWLDDQPTKSVIFLCFGSFGSFGVPQLREIAIGLE
QSGARFLWSIRQAKSIFEMPIECEKPEEFLPQGFLDRTKELGMICGWTPQIEVLAHRAIG
GFVSHCGWNSILESLWHGVPIVTWPLYAEQQINAFQIVKDLGLGVELRLDYRRDGGDVDV
VMRDEIEKAVRYVMDGDSEVRQRVQEASEKCRKAVVEGGSSFSSIGSLIEAILANIS*

### Pharmaceutical Compositions

In one aspect, provided herein is a pharmaceutical composition comprising a compound described herein (e.g., a compound of Formula (I) and other formulas described herein) or a pharmaceutically acceptable salt thereof, and a pharmaceutically acceptable excipient. In certain embodiments, the compound of the present invention is provided in an effective amount in the pharmaceutical composition. In certain embodiments, the compound of the present invention is provided in a therapeutically effective amount. In certain embodiments, the compound of the present invention is provided in a prophylactically effective amount.

In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, inhalation, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

In another aspect, the invention provides a pharmaceutical composition comprising a compound of the present invention and a pharmaceutically acceptable excipient, e.g., a composition suitable for injection, such as for intravenous (IV) administration.

Pharmaceutically acceptable excipients include any and all diluents or other liquid vehicles, dispersion or suspension aids, surface active agents, isotonic agents, preservatives, lubricants and the like, as suited to the particular dosage form desired, e.g., injection. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

For example, injectable preparations, such as sterile injectable aqueous suspensions, can be formulated according to the known art using suitable dispersing or wetting agents and suspending agents. Exemplary excipients that can be employed include, but are not limited to, water, sterile saline or phosphate-buffered saline, or Ringer's solution.

The injectable composition can be sterilized, for example, by filtration through a bacterial-retaining filter, or by incorporating sterilizing agents in the form of sterile solid compositions which can be dissolved or dispersed in sterile water or other sterile injectable medium prior to use.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, response of the individual patient, the severity of the patient's symptoms, and the like.

In some embodiments, the amount of the compound (e.g., the compound of a compound of Formula (I), Formula (Ia), Formula (Ib), Formula (Ibb), Formula (Ic), Formula (Ie), Formula (Ie-α), or Formula (Ie-β), or a subformula thereof), or a pharmaceutically acceptable salt thereof, in a pharmaceutical composition described herein is about 5 mg to about 800 mg, about 10 mg to about 800 mg, about 15 mg to about 800 mg, about 20 mg to about 800 mg, about 25 mg to about 800 mg, about 30 mg to about 800 mg, about 35 mg to about 800 mg, about 40 mg to about 800 mg, about 45 mg to about 800 mg, about 45 mg to about 800 mg, about 50 mg to 800 mg, about 55 mg to about 800 mg, about 60 mg to about 800 mg, about 65 mg to about 800 mg, about 70 mg to about 800 mg, about 75 mg to about 800 mg, about 80 mg to about 800 mg, about 85 mg to about 90 mg, about 95 mg to about 800 mg, about 100 mg to about 800 mg, about 125 mg to about 800 mg, about 150 mg to about 800 mg, about 175 mg to about 800 mg, about 200 mg to about 800 mg, about 225 mg to about to about 800 mg, about 250 mg to about 800 mg, about 275 mg to about 800 mg, about 300 mg to about 800 mg, about 325 mg to about 800 mg, about 350 mg to about 800 mg, about 375 mg to about 800 mg, about 400 mg to about 800 mg, about 425 mg to about 800 mg, about 450 mg to about 800 mg, about 475 mg to about 800 mg, about 500 mg to about 800 mg, about 550 mg to about 800 mg, about 600 mg to about 800 mg, about 650 mg to about 800 mg, about 700 mg to about 800 mg, about 750 mg to about 800 mg, about 5 mg to about 10 mg, about 5 mg to about 15 mg, about 5 mg to about 20 mg, about 5 mg to about 25 mg, about 5 mg to about 30 mg, about 5 mg to about 35 mg, about 5 mg to about 40 mg, about 5 mg to about 45 mg, about 5 mg to about 50 mg, about 5 mg to about 55 mg, about 5 mg to about 60 mg, about 5 mg to about 65 mg, about 5 mg to about 70 mg, about 5 mg to about 75 mg, about 5 mg to about 80 mg, about 5 mg to about 85 mg, about 5 mg to about 90 mg, about 5 mg to about 95 mg, about 5 mg to about 100 mg, about 5 mg to about 125 mg, about 5 mg to about 150 mg, about 5 mg to about 175 mg, about 5 mg to about 200 mg, about 5 mg to about 225 mg, about 5 mg to about 250 mg, about 5 mg to about 250 mg, about 5 mg to about 275 mg, about 5 mg to about 300 mg, about 5 mg to about 325 mg, about 5 mg to about 350 mg, about 5 mg to about 375 mg, about 5 mg to about 400 mg, about 5 mg to about 425 mg, about 5 mg to about 450 mg, about 5 mg to about 475 mg, about 5 mg to about 500 mg, about 5 mg to about 505 mg, about 5 mg to about 600 mg, about 5 mg to about 650 mg, about 5 mg to about 700 mg, about 5 mg to about 750 mg, about 5 mg to about 50 mg, about 25 mg to about 75 mg, about 50 mg to about 100 mg, about 100 mg to about 250 mg, about 200 mg to about 400 mg, about 300 mg to about 600 mg, or about 500 mg to about 750 mg.

The compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampules or syringes of the liquid compositions. In such compositions, the compound is usually a minor component (from about 0.1% to about 50% by weight or preferably from about 1% to about 40% by weight) with the remainder being various vehicles or carriers and processing aids helpful for forming the desired dosing form.

The compounds provided herein can be administered as the sole active agent, or they can be administered in combination with other active agents. In one aspect, the present invention provides a combination of a compound of the present invention and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative oxysterols that have been listed herein. The compounds provided herein may be prepared from known or commercially available starting materials and reagents by one skilled in the art of organic synthesis. Exemplary chiral columns available for use in the separation/purification of the enantiomers/diastereomers provided herein include, but are not limited to, CHIRALPAK® AD-10, CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

¹H-NMR reported herein (e.g., for the region between δ (ppm) of about 0.5 to about 4 ppm) will be understood to be an exemplary interpretation of the NMR spectrum (e.g., exemplary peak integratations) of a compound. Exemplary general method for preparative HPLC: Column: Waters rBridge prep 10 □m C18, 19*250 mm. Mobile phase: acetonitrile, water (NH₄HCO₃) (30 L water, 24 g NH₄HCO₃, 30 mL NH₃.H₂O). Flow rate: 25 mL/min.

Exemplary general method for analytical HPLC: Mobile phase: A: water (10 mM NH₄HCO₃), B: acetonitrile Gradient: 5%-95% B in 1.6 or 2 min Flow rate: 1.8 or 2 mL/min; Column: xBridge C18, 4.6*50 mm, 3.5 □m at 45 C.

Exemplary general method for SFC: Column: CHIRALPAK® AD CSP (250 mm*30 mm, 10 μm), Gradient: 45% B, A=NH₃H₂O, B=MeOH, flow rate: 60 mL/min. For example, AD_3_EtOH_DEA_5_40_25ML would indicate: Column: Chiralpak AD-3 150×4.6 mm I.D., 3 μm Mobile phase: A: CO2 B: ethanol (0.05% DEA) Gradient: from 5% to 40% of B in 5 min and hold 40% for 2.5 min, then 5% of B for 2.5 min Flow rate: 2.5 mL/min Column temp: 35° C.

### Example 1. Preparation of Glycosylated 4-HO-MET Compounds

4-HO-MET was dissolved in 100% (v/v) DMSO to a concentration of 10 mM. The solution was stored at −20° C. prior to use.

FastAP phosphatase (1 U/uL) from ThermoFisher Scientific (catalog no. EF0651).

GLY enzume suspensions contain 10 mM Tris-HCl at pH7.4, 250 mM imidazole and 50% (v/v) glycerol.

The assay conditions were:
100 mM Tris-HCl, pH 7.4
5 mM MgCl2
1 mM KCl
1.25 mM UDP-glucose
0.5 mM 4-OH-MET
5% (v/v) DMSO
0.1 U/uL Fast AP phosphatase
25% (v/v) GLY enzyme

Incubation was 24 house at 30° C. Reactions were terminated adding 3 volumes of 75% (v/v) ethanol.

LC separation was performed using an Agligen 1290 Infinity II UHPLC equipped with a Phenomenex Kinetex XB-C18 column (1.7 μm, 100 angstroms, 100×2.1 mm) and 4-HO-MET was detected by absorbance at 210 nm and 268 nm with an Agilent 1260 Infinity II DAD.

0.1% Formic acid in water (A) and 0.1% Formic acid in acetonitrile (B) were used as the mobile phases for the elution gradient, with a flow rate of 0.4 ml/min. Retention time for 4-HO-MET using this type of instrument under these condition was 2.6 min. The system was coupled to an Ultivo Triple Quadrupole MS. To verify the presence of possible glycoside products, as SIM (Selected Ion Monitoring) method was used on the MS to analyse for the m/z 381 (theoretical mass to chart ratio of 4-HO-MET-glucoside) and m/z 219 (theoretical mass to charge ratio of 4-HO-MET).

As standard, 4-HO-MET solutions at a concentrations of 500, 250, 125, 62.5, and 31.25 uM were prepared from the 10 mM stock and used for calibration.

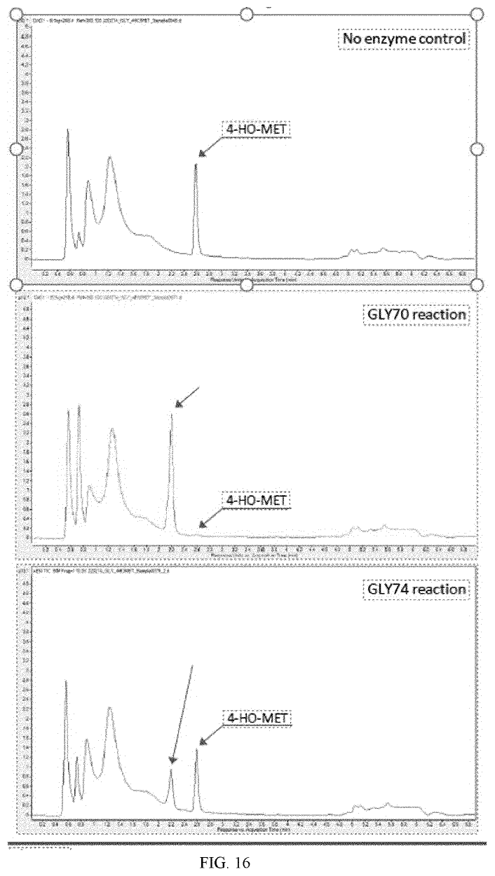

A representative example of the chromatograms obtained with a wavelength of 268 nm for the standard solutions resuspended in the reaction buffer, and the corresponding MS-SIM signal, is shown in FIG. 16. The peak corresponding to 4-HO-MET is indicated.

When analyzing the GLY reactions, 30 of them gave an additional peak with retention time 2.2 min (i.e. in the expected region for a 4-HO-MET glucoside) and molecular mass compatible with the expected glucoside product, together with a corresponding decrease in substrate concentration, compared to the no-enzyme controls. Representative chromatograms are shown in FIG. 16.

The method was repeated using various GLY enzymes to produce 4-HO-MET glucosides.

### Example 2. Percentage of Converted 4-HO-MET

The enzymes giving the additional peak putatively corresponding to the 4-HO-MET-B-D-glucoside are listed below. Peak areas in the UV chromatograms suggest different efficiencies for the different enzymes. A correct quantification of the product is not possible without a standard for the specific molecule. However, based on the assumption that the ratio area-to-concentration in the UV chromatograms will be similar to 4-HO-MET, estimates of the relative abundance for the additional peak are also given, expressed as percentage of converted 4-HO-MET. GLY enzymes hits are ranked according to their estimated conversion efficiency.

| UGT | 4-OH-MET GLY % |
|---|---|
| GLY70 | 98.6 |
| GLY87 | 95.2 |
| GLY77 | 66.5 |
| GLY169 | 52.3 |
| GLY163 | 40.5 |
| GLY74 | 36 |
| GLY170 | 31.6 |
| GLY91 | 20.7 |
| GLY164 | 10.2 |
| GLY108 | 8.7 |
| GLY73 | 7.4 |
| GLY153 | 6 |
| GLY119 | 6 |

### Example 3. Percentage of Converted 4-OH-EPT

The experiments described in Examples 1 and 2 above were repeated for 4-OH-EPT.

Below is the corresponding data for 4-OH-EPT.

| UGT | 4-OH-EPT GLY % |
|---|---|
| GLY70 | 80.5 |
| GLY87 | 68.9 |
| GLY77 | 66.2 |
| GLY163 | 46.3 |
| GLY169 | 33 |
| GLY170 | 23.4 |
| GLY74 | 7 |
| GLY91 | 5.4 |
| GLY146 | 5.3 |

### Example 4. Percentage of Converted 4-OH-MiPT

The experiments described in Examples 1 and 2 above were repeated for 4-OH-MiPT, 4-HO-DMT and 5-HO-DMT

Below is the corresponding data for 4-OH-MiPT.

| UGT | 4-OH-MiPT GLY % |
|---|---|
| GLY77 | 85 |
| GLY87 | 76.4 |
| GLY169 | 71.5 |
| GLY163 | 61.6 |
| GLY70 | 58.4 |
| GLY170 | 38.8 |
| GLY146 | 23.2 |
| GLY74 | 19.3 |

Below is the corresponding data for 4-OH-DMT and 5-HO-DMT.

| UGT | 4-OH-DMT GLY % | UGT | 5-OH-DMT GLY % |
|---|---|---|---|
| GLY70 | 89.3 | GLY91 | 1 |
| GLY87 | 85.3 | GLY167 | 2 |
| GLY77 | 30.2 | GLY87 | 3 |
| GLY169 | 25.0 | GLY89 | 4 |
| GLY74 | 22.4 | GLY153 | 5 |
| GLY170 | 15.7 | GLY70 | 6 |
| GLY73 | 14.0 | GLY63 | |
| GLY791 | 11.3 | GLY67 | |
| GLY163 | 10.6 | GLY68 | |
| GLY76 | 9.5 | GLY69 | |

### Example 5. NMR Analysis for 4-HO-MET Glycoside

A total of 10 mg of Sample 4 (4 HO Met glycoside) was dissolved in 100 uL deuterated water (containing DSS as internal standard) and 30 μL of this solution was transferred to 1.7 mm o.d. NMR tubes. NMR experiments were acquired at 300 K on a Bruker Avance III spectrometer (1H resonance frequency 600.13 MHz) equipped with a cryogenically cooled gradient inverse triple-resonance 1.7-mm TCI probe head optimized for 1H and 13C observation and a SampleJet sample changer (Bruker BioSpin GmbH, Rheinstetten, Germany). Acquisition of data was performed using TopSpin ver. 3.2, and automation was controlled by IconNMR ver. 4.2 (Bruker BioSpin GmbH, Rheinstetten, Germany). Data processing was performed using TopSpin ver. 4.02 for Mac (Bruker BioSpin GmbH, Rheinstetten, Germany). 1H chemical shifts were referenced to the DSS at δ 0.0). 1H-NMR spectra were acquired in automation (temperature equilibration to 300 K, optimization of lock parameters, gradient shimming, and setting of receiver gain) using the standard zg30 pulse sequence (Bruker library) collecting 64 k data points over a spectral width of 12,019 Hz (20.03 ppm) and adding 128 transients. Phase-sensitive DQF-COSY spectra were recorded using a gradient-based pulse sequence with a 8.00 ppm spectral width and 2k×512 data points (processed with forward linear prediction to 1 k data points). Phase-sensitive NOESY and ROESY spectra were recorded using with spectral widths of 8.00 ppm in both dimensions and 2 k×256 data points (processed with forward linear prediction to 1 k data points). For NOESY spectra a mixing time of 600 ms was used. Multiplicity-edited HSQC spectra were acquired with the following parameters: spectral width 12.02 ppm for 1H and 170 ppm for 13C, 1730×512 data points (processed with forward linear prediction to 1 k data points), and 1.0 s relaxation delay. HMBC spectra (with a low-pass filter) were optimized for nJC, H=8 Hz and acquired using the following parameters: spectral width 8.00 ppm for 1H and 220 ppm for 13C, 2 k×512 data points (processed with forward linear prediction to 1 k data points), and 1.0 s relaxation delay.

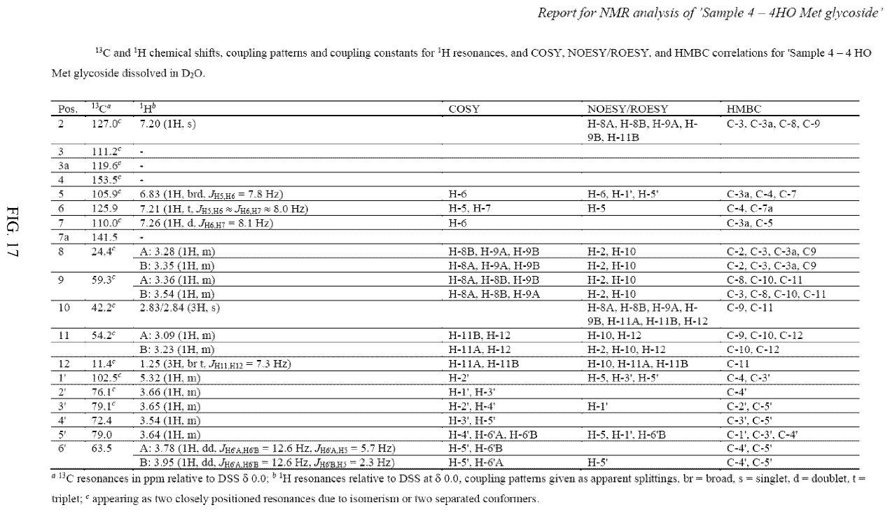

The 1D ¹H NMR, ¹³C/QDEPT NMR as well as 2D DQF-COSY, multiplicity-edited HSQC, ROESY, NOESY (600 ms mixing time), and low-pass filtered HMBC spectra for Sample 4-(4 HO Met glycoside) (hereafter named compound 1a) are provided herein, the structure and numbering is provided in the compound below, and assigned ¹H- and ¹³C NMR data with 2D correlations to support the structure are provided in FIG. 17.

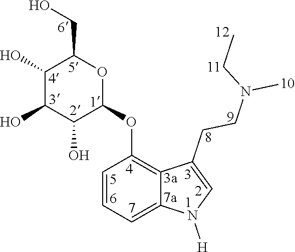 [SMILES: CCN(C)CCc1c[nH]c2cccc(O[C@@H]3O[C@H](CO)[C@@H](O)[C@H](O)[C@H]3O)c12]

The DQF-COSY spectrum showed spin systems corresponding to H-5↔H-6↔H-7, H-8A/H-8B↔H-9A/H-9B, H-11A/H11B↔H-H-12, and H-1′↔H-2′↔H-3′↔H-4′↔H-5′↔H-6′A/H-6′B in agreement with the core skeleton of 1. The 1H and 13C NMR chemical shift values of the glycoside is in agreement for those observed from beta-D-glucopyranoside. HMBC correlations from H-1′ to C-4 established the glucose moiety to be attached to C-4, and this was further supported by NOESY and ROESY correlations between H-5 and Hl′. HMBC correlations from to H-9 and H-10 to C-11, from H-10 and H-11 to C-9, and from H-9 and H-11 to C-10 established structure of the (ethyl(methyl)amino) ethyl group, and its attachment to C-3 was established by HMBC correlations from H-2 to C-8 and C-9 and from H-8A/H-8B to C-2, C-3, and C-3a as well NOESY and ROESY correlations between H-2 and H-8A/H-8B. Selected HMBC correlations (arrows pointing from H to C) and selected NOESY/ROESY correlations are shown below.

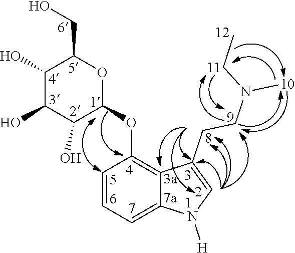 [SMILES: CCN(C)CCc1c[nH]c2cccc(O[C@@H]3O[C@H](CO)[C@@H](O)[C@H](O)[C@H]3O)c12]

Full analysis of all 2D NMR spectra supported the structure of 1, and all 2D NMR correlations from COSY, NOESY/ROESY, HSQC, and HMBC are shown in FIG. 17, vide infra. Although the 2D NMR data support the structure of 1, the majority of all 13C NMR resonances were observed as two pairs of closely positioned signals (marked with c in FIG. 17), as is also observed for, e.g., the H-10 methyl singlet. This is most likely due to the peri-positioned beta-D-glucopyranoside and (ethyl(methyl)amino) ethyl groups, causing two conformational isomers due to hindered rotation around the C-4-C-1′ bond. The beta-configuration of the anomeric proton H-1′ is based on its chemical shift value [1], as well as the appearance of of coupling patterns in the range of 7.9 Hz, which is in agreement with the 3JH1′,H2′ coupling constant for 1,2-trans diaxial positioned hydrogens. The glucose unit was tentatively assigned to have the D configuration based on biosynthetic considerations.

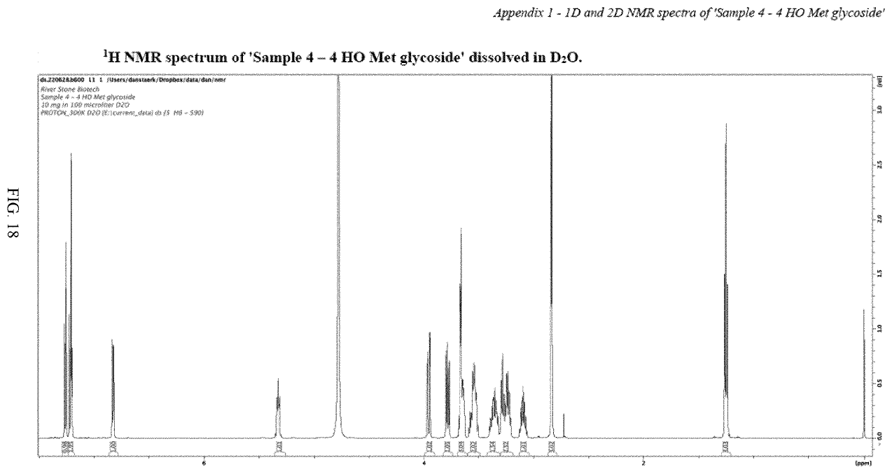

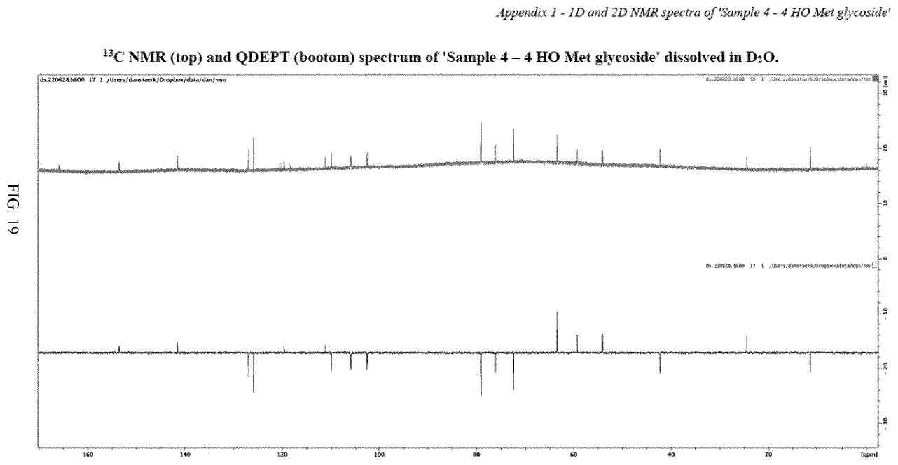

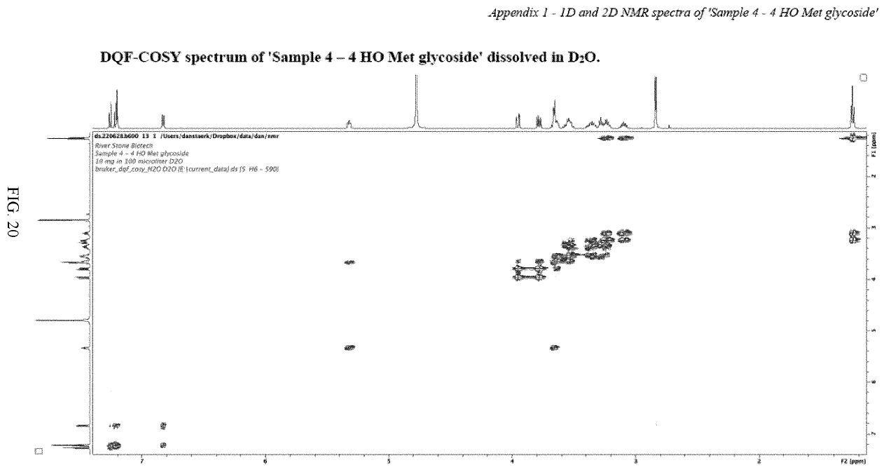

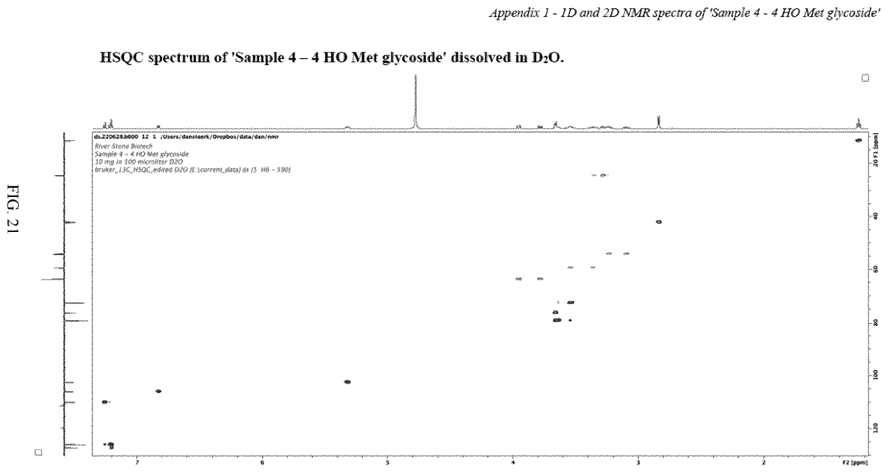

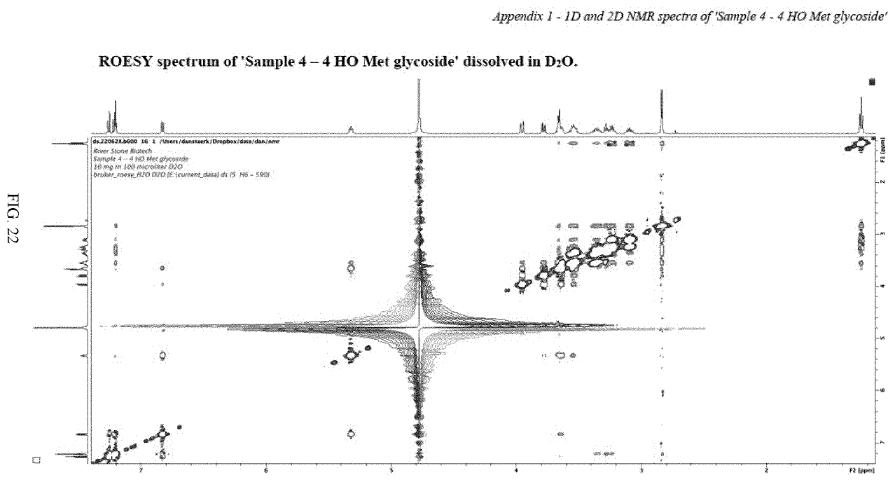

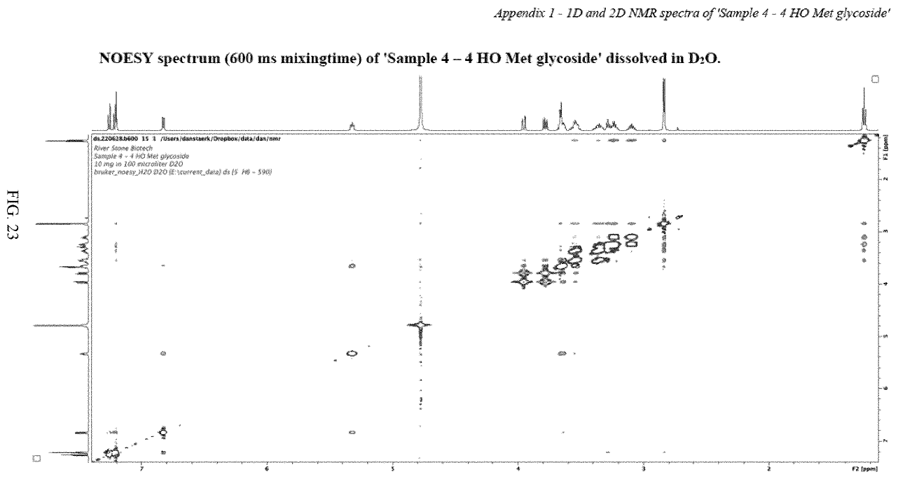

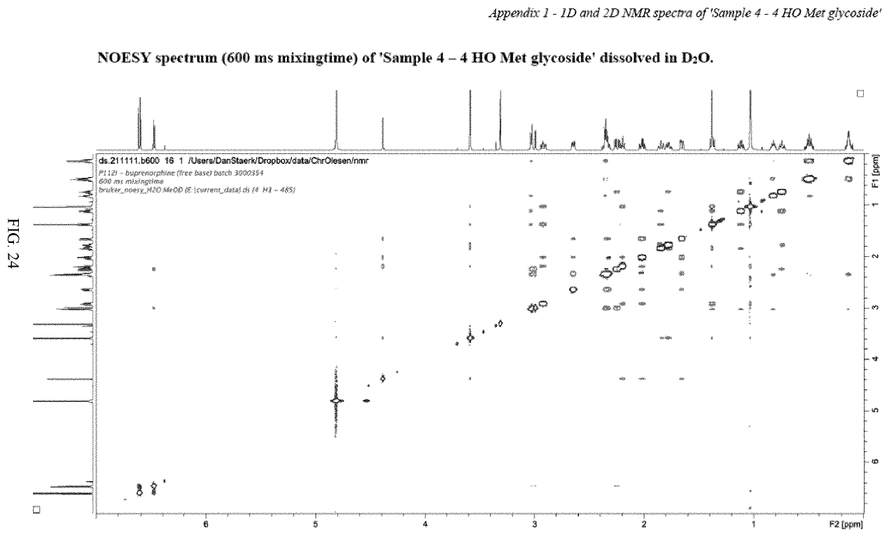

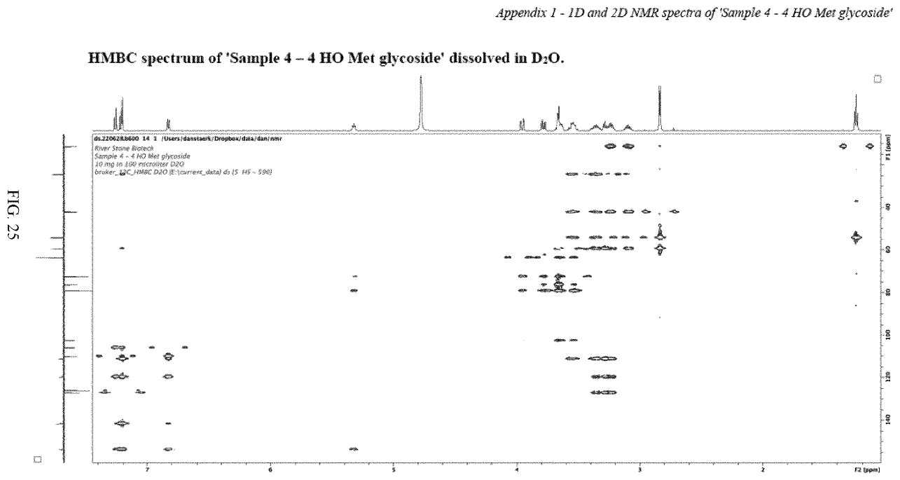

Concerning purity of the delivered sample, signals from other compounds are well below the carbon-13 satellites of the signals of 1a, and thereby well below 0.55% impurity. FIG. 18, FIG. 19, FIG. 20, FIG. 21, FIG. 22, FIG. 23, FIG. 24, and FIG. 25 depict figures of 1D 1H and 13C NMR spectra as well as 2D DQF-COSY (double-quantum filtered correlation spectroscopy), multiplicity-edited HSQC (heteronuclear single quantum coherence), low-pass filtered HMBC (heteronuclear multiple bond correlation), ROESY (rotational Overhauser effect spectroscopy), and NOESY (Nuclear Overhauser effect spectroscopy) with a mixing time of 600 ms of “Sample 4-4 HO Met glycoside” are provided, with the purpose of making a full assignment of all 1H and 13C NMR resonances of this compound.

### Example 6. NMR Analysis for 4-HO-EPT Glycoside

A total of 10 mg 4-HO-EPT glycoside was dissolved in 100 uL deuterated water (containing DSS as internal standard) and 30 μL of this solution was transferred to 1.7 mm o.d. NMR tubes. NMR experiments were acquired at 300 K on a Bruker Avance III spectrometer (1H resonance frequency 600.13 MHz) equipped with a cryogenically cooled gradient inverse triple-resonance 1.7-mm TCI probe head optimized for ¹H and ¹³C observation and a SampleJet sample changer (Bruker BioSpin GmbH, Rheinstetten, Germany). Acquisition of data was performed using TopSpin ver. 3.2, and automation was controlled by IconNMR ver. 4.2 (Bruker BioSpin GmbH, Rheinstetten, Germany). Data processing was performed using TopSpin ver. 4.02 for Mac (Bruker BioSpin GmbH, Rheinstetten, Germany). 1H chemical shifts were referenced to the DSS at δ 0.0). 1H-NMR spectra were acquired in automation (temperature equilibration to 300 K, optimization of lock parameters, gradient shimming, and setting of receiver gain) using the standard zg30 pulse sequence (Bruker library) collecting 64 k data points over a spectral width of 12,019 Hz (20.03 ppm) and adding 128 transients. Phase-sensitive DQF-COSY spectra were recorded using a gradient-based pulse sequence with a 8.00 ppm spectral width and 2 k×512 data points (processed with forward linear prediction to 1 k data points). Phase-sensitive NOESY and ROESY spectra were recorded using with spectral widths of 8.00 ppm in both dimensions and 2 k×256 data points (processed with forward linear prediction to 1 k data points). For NOESY spectra a mixing time of 600 ms was used. Multiplicity-edited HSQC spectra were acquired with the following parameters: spectral width 12.02 ppm for 1H and 170 ppm for 13C, 1730×512 data points (processed with forward linear prediction to 1 k data points), and 1.0 s relaxation delay. HMBC spectra (with a low-pass filter) were optimized for nJC, H=8 Hz and acquired using the following parameters: spectral width 8.00 ppm for 1H and 220 ppm for 13C, 2 k×512 data points (processed with forward linear prediction to 1 k data points), and 1.0 s relaxation delay.

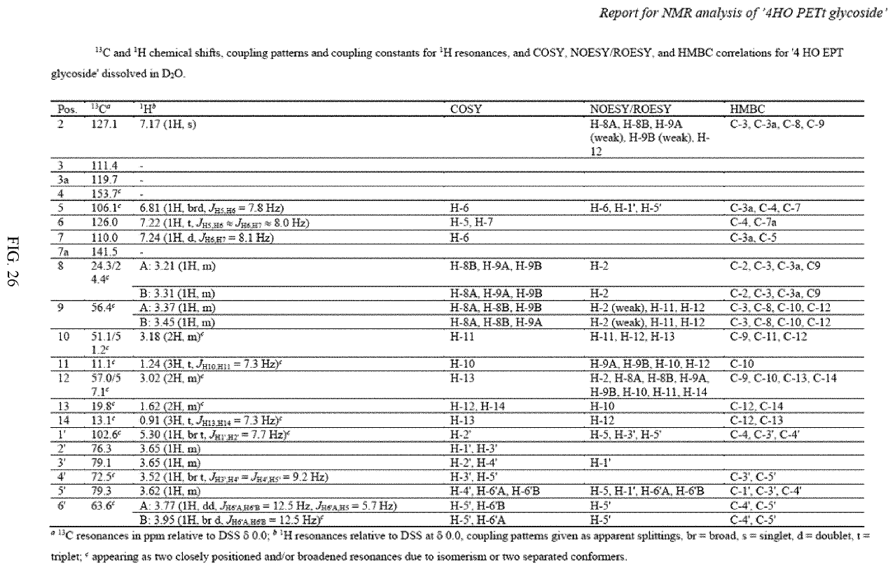

The 1D ¹H NMR and ¹³C/QDEPT NMR as well as 2D DQF-COSY, multiplicity-edited HSQC, ROESY, NOESY (600 ms mixing time), and low-pass filtered HMBC spectra for 4-HO-EPT glycoside (hereafter named compound 1b) are provided herein, the structure and numbering is provided in the compound below, and assigned ¹H- and ¹³C NMR data with 2D correlations to support the structure are provided in FIG. 26.

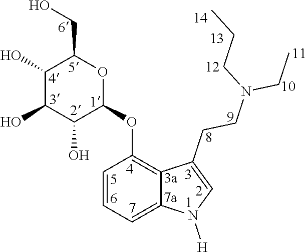 [SMILES: CCCN(CC)CCc1c[nH]c2cccc(O[C@@H]3O[C@H](CO)[C@@H](O)[C@H](O)[C@H]3O)c12]

The DQF-COSY spectrum showed spin systems corresponding to H-5↔H-6↔H-7, H-8A/H-8B↔H-9A/H-9B, H-10↔H-11, H-12↔H-13↔H-14, and H-1′↔H-2′↔H-3′↔H-4′↔H-5′↔H-6′A/H-6′B in agreement with the core skeleton of 1b. The 1H and 13C NMR chemical shift values of the glycoside is in agreement for those observed from β-D-glucopyranoside. HMBC correlations from H-1′ to C-4 established the glucose moiety to be attached to C-4, and this was further supported by NOESY and ROESY correlations between H-5 and H1′. HMBC correlations from to H-9 and H-10 to C-12, from H-9 and H-12 to C-10, and from H-10 and H-12 to C-9 established structure of the (ethyl (propyl) amino) ethyl group, and its attachment to C-3 was established by HMBC correlations from H-2 to C-8 and C-9 and from H-8A/H-8B to C-2, C-3, and C-3a as well NOESY and ROESY correlations between H-2 and H-8A/H-8B. Selected HMBC correlations (arrows pointing from H to C) and selected NOESY/ROESY correlations are shown below.

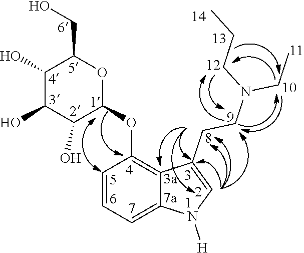 [SMILES: CCCN(CC)CCc1c[nH]c2cccc(O[C@@H]3O[C@H](CO)[C@@H](O)[C@H](O)[C@H]3O)c12]

Full analysis of all 2D NMR spectra supported the structure of 1b, and all 2D NMR correlations from COSY, NOESY/ROESY, HSQC, and HMBC are shown in FIG. 26, vide infra. Although the 2D NMR data support the structure of 1b, many ¹³C NMR resonances, i.e., C-4, C-5, C-8, C-10, C-13, C-14, C-1′, and C-6′, were observed as two pairs of closely positioned signals (marked with c in FIG. 26). The appearance of broadened and/or tow closely positioned but overlapping signals are also observed for, e.g., H-1′, H-6′b, H-10, H-11, H-12, H-13, and H-14. This is most likely due to the peri-positioned beta-D-glucopyranoside and (ethyl(propyl)amino) ethyl groups, causing two conformational isomers due to hindered rotation around the C-4-C-1′ bond. The beta-configuration of the anomeric proton H-1′ is based on its chemical shift value [1], as well as the appearance of coupling patterns in the range of 7.9 Hz, which is in agreement with the ³J_{H1′,H2′} coupling constant for 1,2-trans diaxial positioned hydrogens. The glucose unit was tentatively assigned to have the D configuration based on biosynthetic considerations.

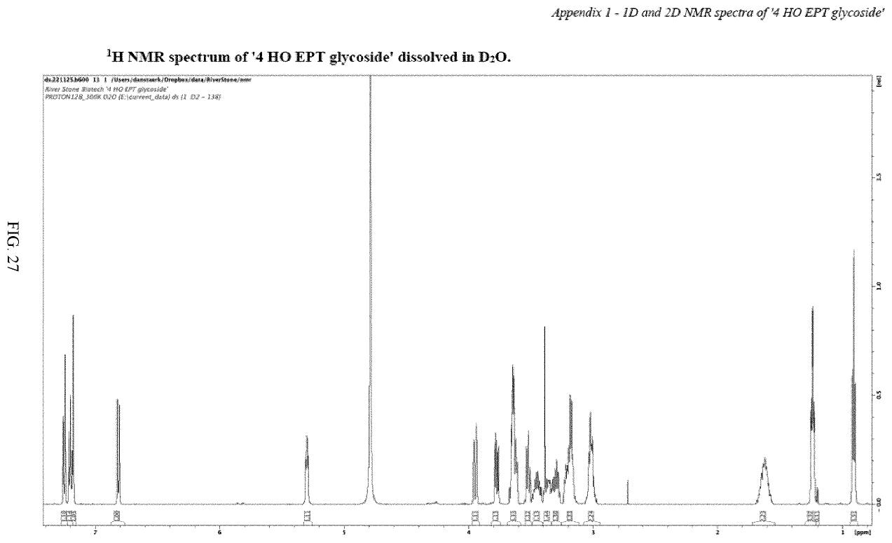

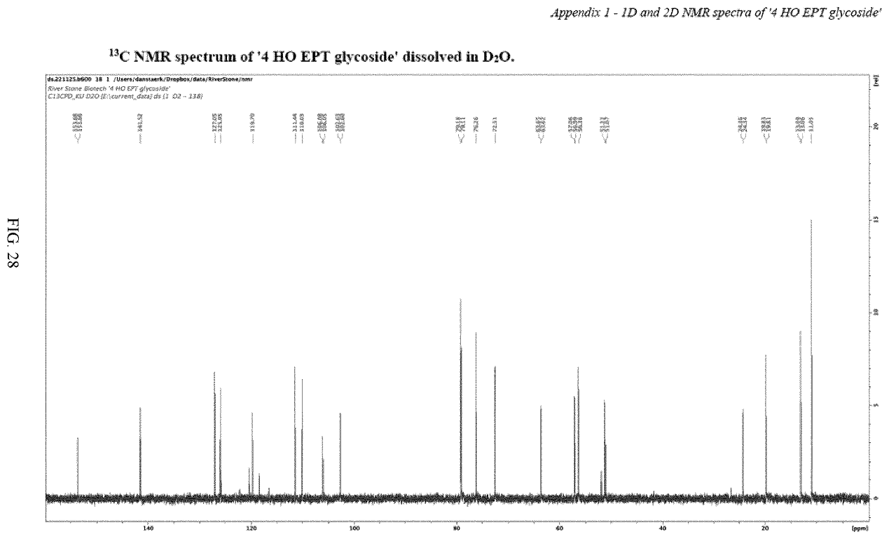

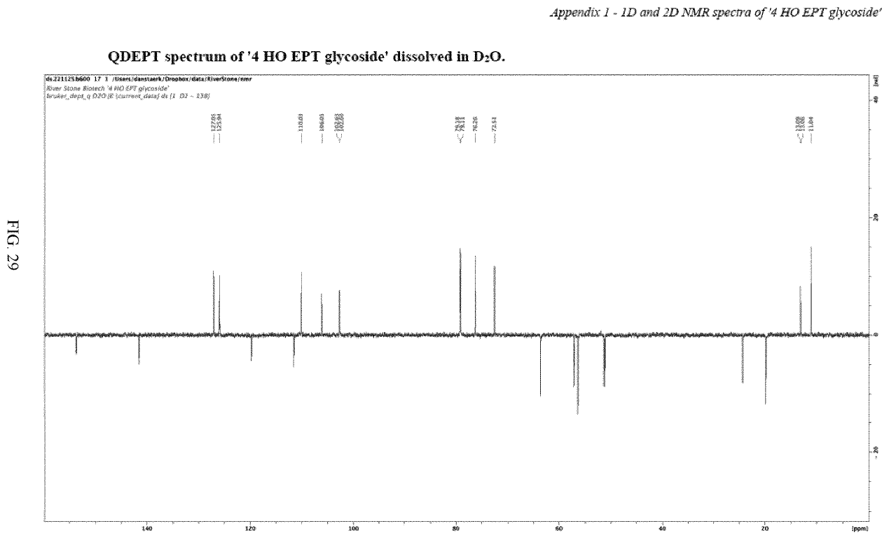

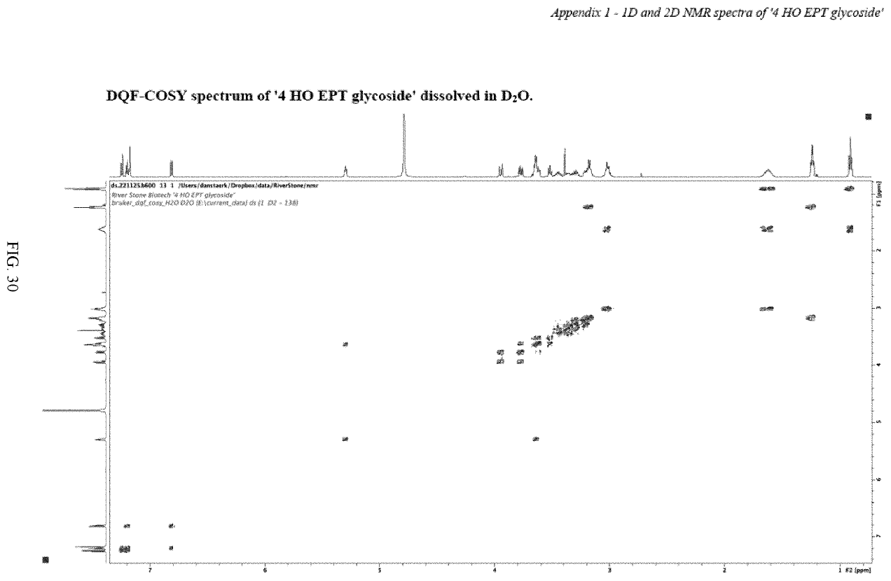

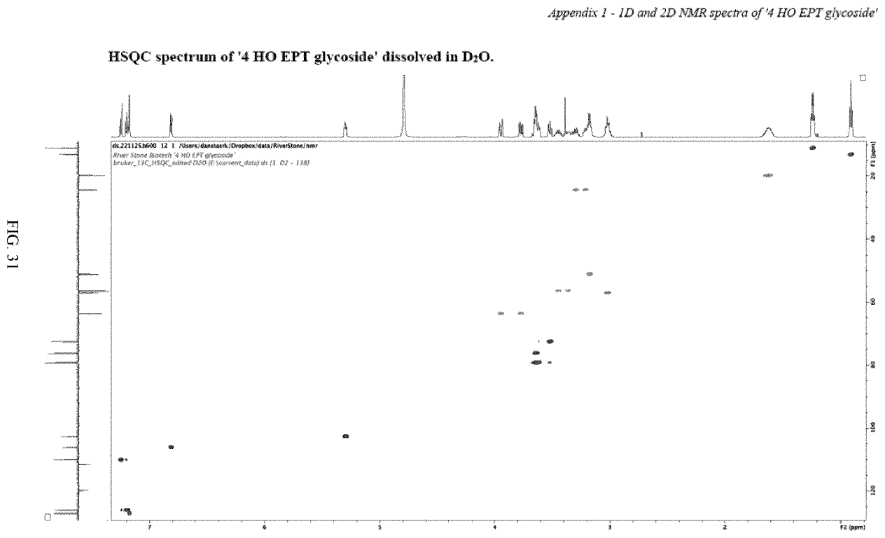

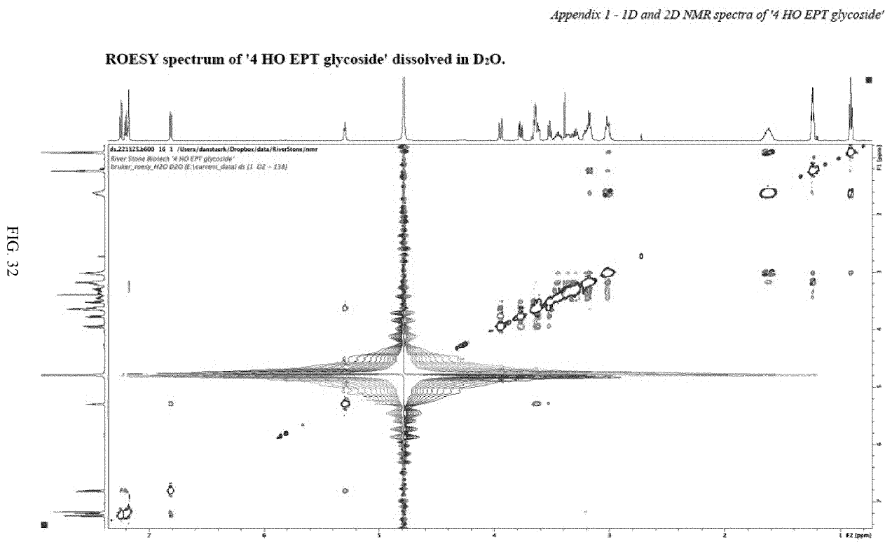

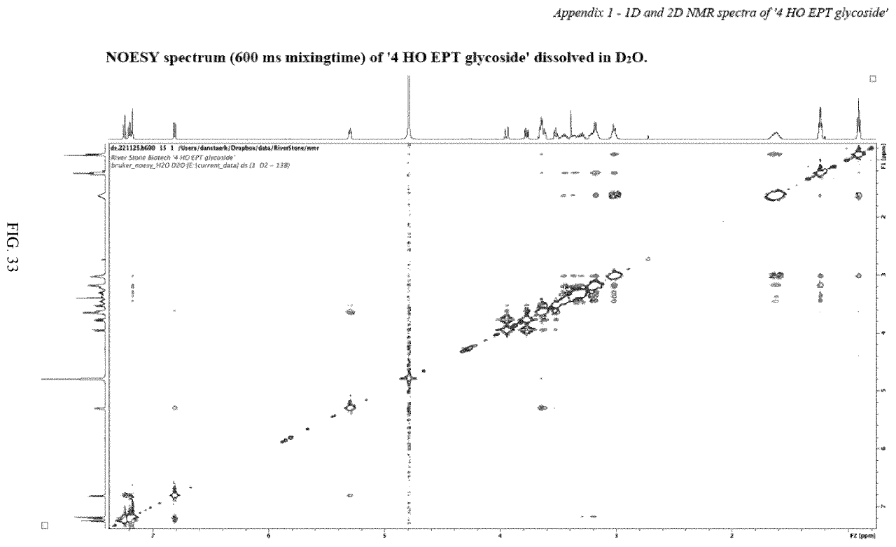

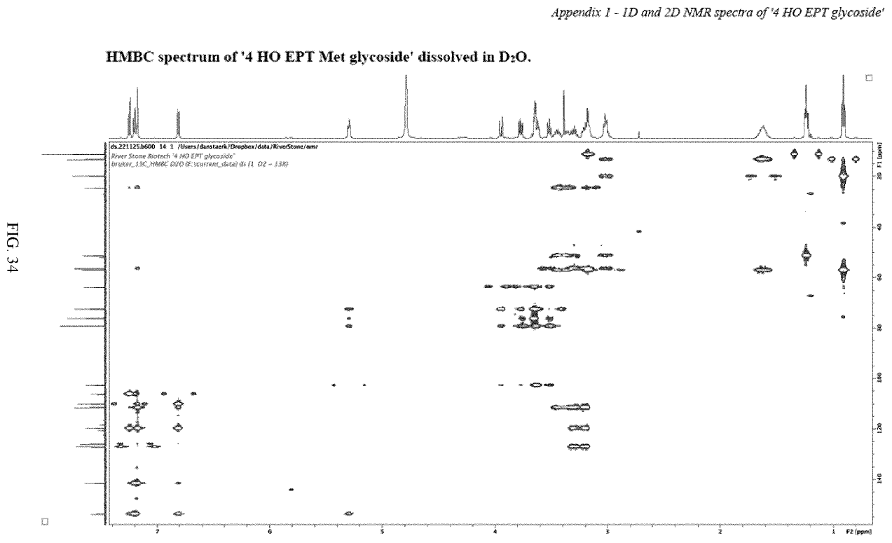

Concerning purity of the delivered sample, signals from other compounds are in the range of 2-3% FIG. 27, FIG. 28, FIG. 29, FIG. 30, FIG. 31, FIG. 32, FIG. 33, and FIG. 34 depict figures of 1D ¹H and ¹³C/QDEPT NMR spectra as well as 2D DQF-COSY (double-quantum filtered correlation spectroscopy), multiplicity-edited HSQC (heteronuclear single quantum coherence), low-pass filtered HMBC (heteronuclear multiple bond correlation), ROESY (rotational Overhauser effect spectroscopy), and NOESY (Nuclear Overhauser effect spectroscopy) with a mixingtime of 600 ms of “4 HO EPT glycoside” are provided, with the purpose of making a full assignment of all 1H and 13C NMR resonances of this compound.

### Example 7. Pharmacokinetics

In a pharmacokinetic experiment in C57Bl/6J mice (3 animals per timepoint; schedule shown below) two example compounds, Cy1 (4-HO-EPT glycoside) and Cy2 (4-HO-DMT glycoside) were dosed at two different concentrations (20 mg/kg dose shown here) and the parent compounds and their de-glycosylated forms, 4-HO-EPT and psilocin (4-HO-DMT), respectively, were measured by LC-MS/MS in plasma and brain tissue samples. For comparison, psilocybin was dosed at 1.5 mg/kg orally and plasma and brain tissue concentrations of psilocin (4-HO-DMT) were measured. Samples were collected at four or five different timepoints (from 10-minutes post dosing up to 6-hours post dosing) following the test article administration.

Schedule of pharmacokinetic experiment shown below.

Results are reported in ng/mL for the concentration in plasma and in ng/g tissue for the concentration measured in brain samples.

An LLOQ of 1.50 ng/g was achieved for Cy1, Psilocin and 4-HO-EPT and 15.0 ng/g for Cy2 in brain tissue homogenate. An LLOQ of 0.50 ng/mL was achieved for all analytes in plasma.

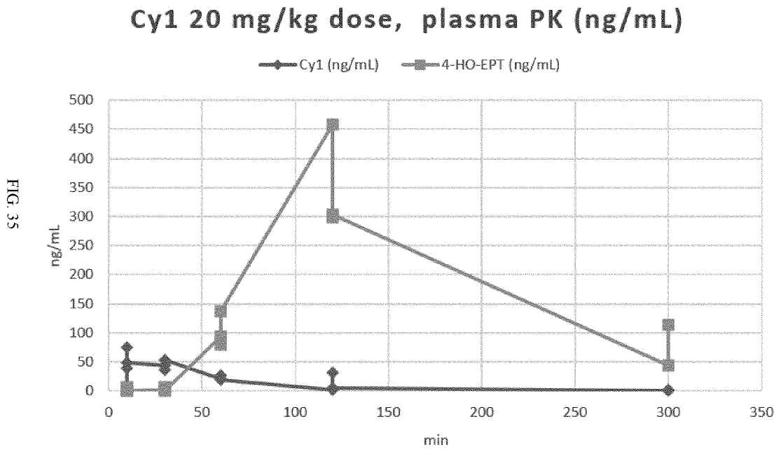

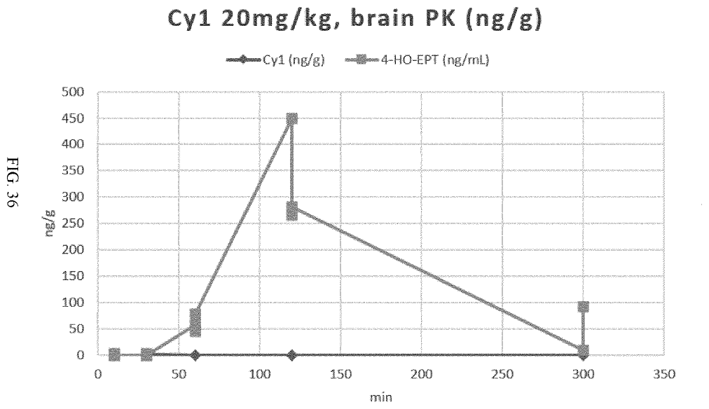

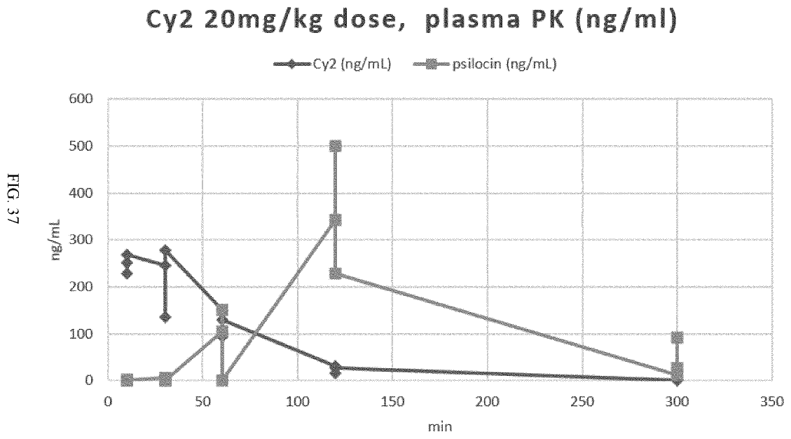

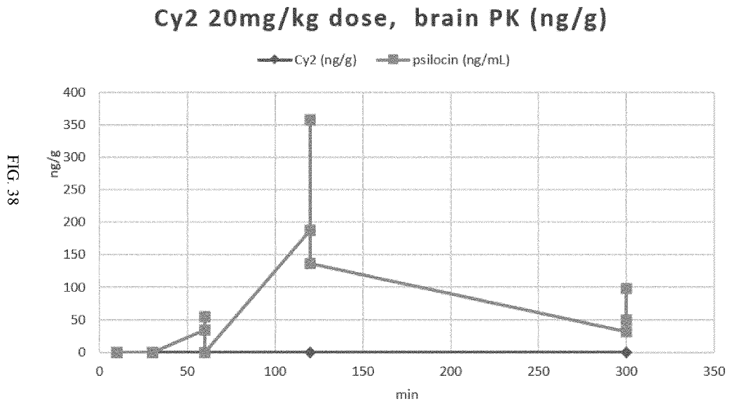

Results: The graphs in FIG. 35, FIG. 36, FIG. 37, and FIG. 38 display the time course of concentrations of compounds as indicated. For each time point the data for the individual animals are plotted.

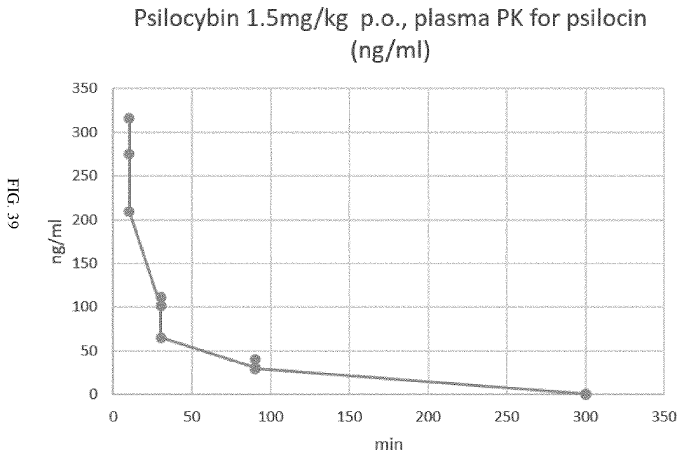

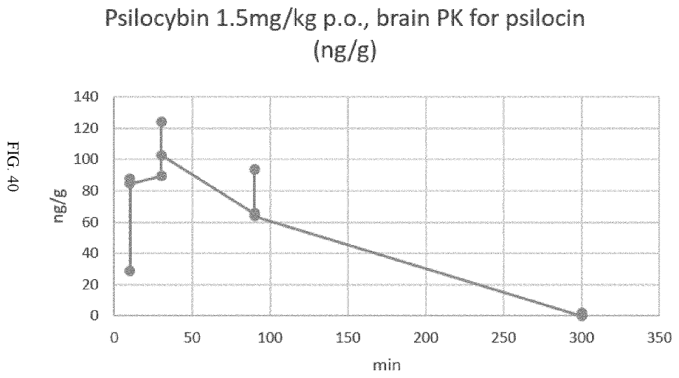

The time to peak plasma and peak brain concentrations is extended after administration of Cy1 and Cy2 as compared to the administration of psilocybin. For Cy2 and psilocybin, the comparison of two molecules both being converted to psilocin can be made. Also, there is an extended period of exposure after administration of higher doses of Cy1 and Cy2 as compared to psilocybin for both plasma and brain (FIG. 39 and FIG. 40).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed:

1. A process of preparing a compound of Formula (I): 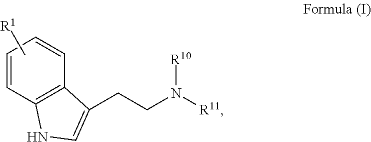
or a salt thereof, comprising:
glycosylating the hydroxy group of a compound of Formula (I-1): 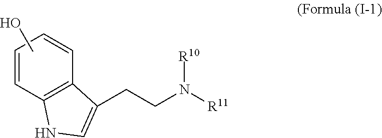
or a salt thereof, thereby producing the compound of Formula (I),
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl; and
wherein glycosylating the hydroxy group of the compound of Formula (I-1) comprises contacting the compound of Formula (I-1) with a glycosyltransferase,
wherein the glycosyltransferase is GLY70, GLY87, GLY77, GLY169, GLY163, GLY74, GLY170, GLY91, GLY164, GLY108, GLY146, GLY119, GLY73, or GLY153.

2. A process of preparing a compound of Formula (Ia): 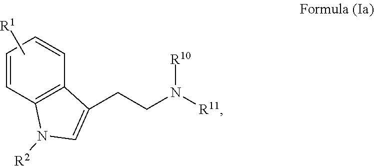
or a salt thereof, comprising:
glycosylating the hydroxy group of a compound of Formula (Ia-1): 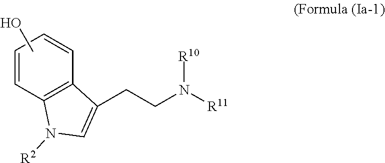
or a salt thereof, thereby producing the compound of Formula (Ia),
wherein
R¹ is a glycosyl group;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl; and
wherein glycosylating the hydroxy group of the compound of Formula (Ia-1) comprises contacting the compound of Formula (Ia-1) with a glycosyltransferase,
wherein the glycosyltransferase is GLY70, GLY87, GLY77, GLY169, GLY163, GLY74, GLY170, GLY91, GLY164, GLY108, GLY146, GLY119, GLY73, or GLY153.

3. A process of preparing a compound of Formula (Ib): 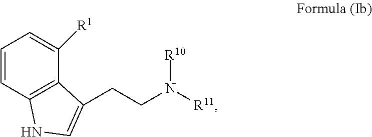
or a salt thereof, comprising:
glycosylating the hydroxy group of a compound of Formula (Ib-1): 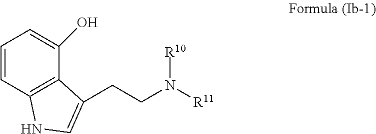
or a salt thereof, thereby producing the compound of Formula (Ib),
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl; and
wherein glycosylating the hydroxy group of the compound of Formula (Ib-1) comprises contacting the compound of Formula (Ib-1) with a glycosyltransferase,
wherein the glycosyltransferase is GLY70, GLY87, GLY77, GLY169, GLY163, GLY74, GLY170, GLY91, GLY164, GLY108, GLY146, GLY119, GLY73, or GLY153.

4. A process of preparing a compound of Formula (Ibb): 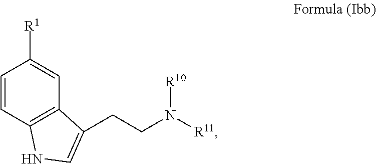
or a salt thereof, comprising:
glycosylating the hydroxy group of a compound of Formula (Ibb-1): 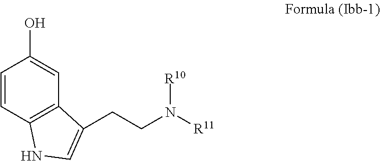
or a salt thereof, thereby producing the compound of Formula (Ibb),
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl; and
wherein glycosylating the hydroxy group of the compound of Formula (Ibb-1) comprises contacting the compound of Formula (Ibb-1) with a glycosyltransferase,
wherein the glycosyltransferase is GLY70, GLY87, GLY77, GLY169, GLY163, GLY74, GLY170, GLY91, GLY164, GLY108, GLY146, GLY119, GLY73, or GLY153.

5. The process of any one of claims 1-4, wherein R¹⁰ is unsubstituted C₁-C₆ alkyl.

6. The process of any one of claims 1-4, wherein R¹⁰ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

7. The process of any one of claims 1-6, wherein R¹¹ is unsubstituted C₁-C₆ alkyl.

8. The process of any one of claims 1-6, wherein R¹¹ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

9. The method of any one of claims 1-8, where R¹ is an —O-glycosyl group.

10. The method of any one of claims 1-8, where R¹ is an-C-glycosyl group.

11. The method of any one of claims 1-8, wherein the glycosyl group is a glucosyl group, a glucuronic acid group, a galactosyl group, a mannosyl group, a fucosyl group, a xylosyl group, a rhamnosyl group, a glucosaminyl group, or a galactosaminyl group.

12. The method of any one of claims 1-11, wherein the glycosyl group is a glucosyl group.

13. The method of any one of claims 1-12, wherein the glycosyl group is b-D-glucosyl.

14. The method of any one of claims 1-8, wherein the glycosyl group is oxygen linked b-D-glucosyl.

15. The process of any one of claims 1 to 14, wherein glycosylating the hydroxy group of the compound of Formula (I-1), Formula (Ia-1), Formula (Ib-1), or Formula (Ibb-1) comprises contacting the compound of Formula (I-1), Formula (Ia-1), Formula (Ib-1), or Formula (Ibb-1) with UDP-glucose, UDP-galactose, UDP-xylose, UDP-glucuronic acid, GDP-mannose, or GDP-fucose.

16. The process of any one of claims 1 to 15, wherein the percent yield of the compound of Formula (I), Formula (Ia), Formula (Ib), or Formula (Ibb) is greater than 85%, is greater than 90%, is greater than 93%, or is greater than 95%.

17. A method of treating a disease, or symptoms thereof, in a subject in need thereof, comprising administering to the subject an effective amount of a compound of Formula (I): 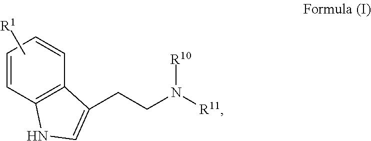
or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

18. A method of treating a disease, or symptoms thereof, in a subject in need thereof, comprising administering to the subject an effective amount of a compound of Formula (Ia): 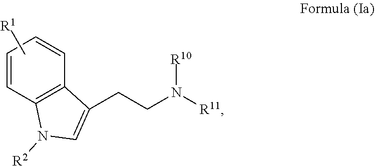
or a pharmaceutically acceptable salt thereof,
wherein
R¹ is a glycosyl group;
R² is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

19. A method of treating a disease or symptoms thereof in a subject in need thereof, comprising administering to the subject an effective amount of a compound of Formula (Ib): 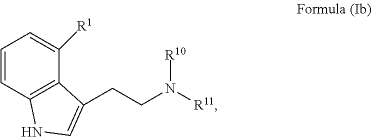
or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

20. A method of treating a disease or symptoms thereof in a subject in need thereof, comprising administering to the subject an effective amount of a compound of Formula (Ibb): 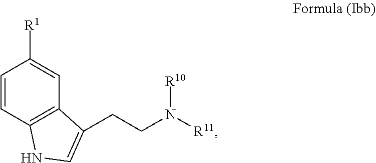
or a pharmaceutically acceptable salt thereof,
wherein
R¹ is a glycosyl group; and
R¹⁰ and R¹¹ are each independently selected from substituted C₁-C₆ alkyl, unsubstituted C₁-C₆ alkyl, substituted C₂-C₆ alkenyl, unsubstituted C₂-C₆ alkenyl, substituted C₃-C₁₀ cycloalkyl, and unsubstituted C₃-C₁₀ cycloalkyl, or
R¹⁰ and R¹¹, together with the nitrogen atom to which they are attached, form a substituted 3- to 10-membered heterocyclyl or an unsubstituted 3- to 10-membered heterocyclyl.

21. The process of any one of claims 17-20, wherein R¹⁰ is unsubstituted C₁-C₆ alkyl.

22. The process of any one of claims 17-20, wherein R¹⁰ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

23. The process of any one of claims 17-22, wherein R¹¹ is unsubstituted C₁-C₆ alkyl.

24. The process of any one of claims 17-22, wherein R¹¹ is methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, or n-hexyl.

25. The method of any one of claims 17-24, where R¹ is an —O-glycosyl group.

26. The method of any one of claims 17-24, where R¹ is an —C-glycosyl group.

27. The method of any one of claims 17-26, wherein the glycosyl group is a glucosyl group, a glucuronic acid group, a galactosyl group, a mannosyl group, a fucosyl group, a xylosyl group, a rhamnosyl group, a glucosaminyl group, or a galactosaminyl group.

28. The method of any one of claims 17-27, wherein the glycosyl group is a glucosyl group.

29. The method of any one of claims 17-28, wherein the glycosyl group is b-D-glucosyl.

30. The method of any one of claims 17-24, wherein the glycosyl group is oxygen linked b-D-glucosyl.

31. The method of any one of claims 17-30, wherein the compound is 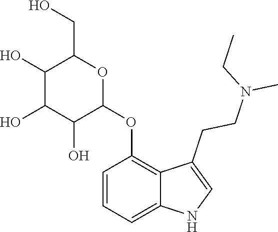 [SMILES: CCN(C)CCc1c[nH]c2cccc(OC3OC(CO)C(O)C(O)C3O)c12]
or a pharmaceutically acceptable salt thereof.

32. The method of any one of claims 17-30, wherein the compound is 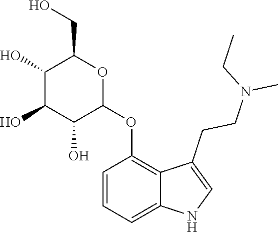 [SMILES: CCN(C)CCc1c[nH]c2cccc(OC3O[C@H](CO)[C@@H](O)[C@H](O)[C@H]3O)c12]
or a pharmaceutically acceptable salt thereof.

33. The method of any one of claims 17-30, wherein the compound is 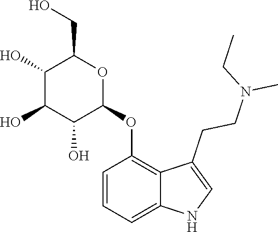 [SMILES: CCN(C)CCc1c[nH]c2cccc(O[C@@H]3O[C@H](CO)[C@@H](O)[C@H](O)[C@H]3O)c12]
or a pharmaceutically acceptable salt thereof.

34. The method of any one of claims 17-30, wherein the compound is 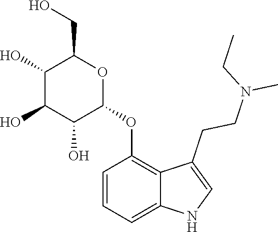 [SMILES: CCN(C)CCc1c[nH]c2cccc(O[C@H]3O[C@H](CO)[C@@H](O)[C@H](O)[C@H]3O)c12]
or a pharmaceutically acceptable salt thereof.

35. The method of any one of claims 17-30, wherein the compound is 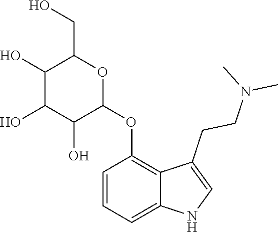 [SMILES: CN(C)CCc1c[nH]c2cccc(OC3OC(CO)C(O)C(O)C3O)c12]
or a pharmaceutically acceptable salt thereof.

36. The method of any one of claims 17-30, wherein the compound is 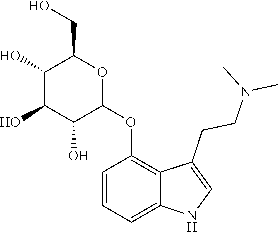 [SMILES: CN(C)CCc1c[nH]c2cccc(OC3O[C@H](CO)[C@@H](O)[C@H](O)[C@H]3O)c12]
or a pharmaceutically acceptable salt thereof.

37. The method of any one of claims 17-30, wherein the compound is 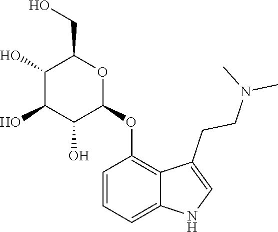 [SMILES: CN(C)CCc1c[nH]c2cccc(O[C@@H]3O[C@H](CO)[C@@H](O)[C@H](O)[C@H]3O)c12]
or a pharmaceutically acceptable salt thereof.

38. The method of any one of claims 17-30, wherein the compound is 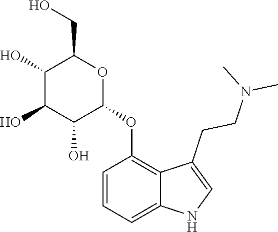 [SMILES: CN(C)CCc1c[nH]c2cccc(O[C@H]3O[C@H](CO)[C@@H](O)[C@H](O)[C@H]3O)c12]
or a pharmaceutically acceptable salt thereof.

39. The method of any one of claims 17-30, wherein the compound is 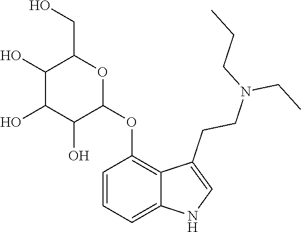 [SMILES: CCCN(CC)CCc1c[nH]c2cccc(OC3OC(CO)C(O)C(O)C3O)c12]
or a pharmaceutically acceptable salt thereof.

40. The method of any one of claims 17-30, wherein the compound is 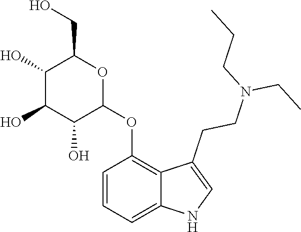 [SMILES: CCCN(CC)CCc1c[nH]c2cccc(OC3O[C@H](CO)[C@@H](O)[C@H](O)[C@H]3O)c12]
or a pharmaceutically acceptable salt thereof.

41. The method of any one of claims 17-30, wherein the compound is 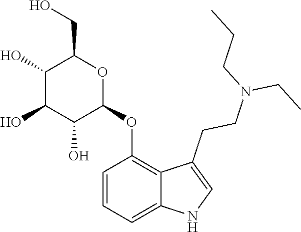 [SMILES: CCCN(CC)CCc1c[nH]c2cccc(O[C@@H]3O[C@H](CO)[C@@H](O)[C@H](O)[C@H]3O)c12]
or a pharmaceutically acceptable salt thereof.

42. The method of any one of claims 17-30, wherein the compound is 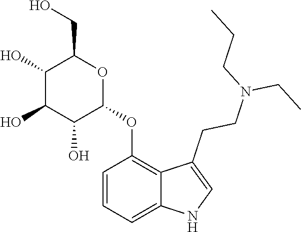 [SMILES: CCCN(CC)CCc1c[nH]c2cccc(O[C@H]3O[C@H](CO)[C@@H](O)[C@H](O)[C@H]3O)c12]
or a pharmaceutically acceptable salt thereof.

43. The method of any one of claims 17-42, wherein the disease is depression, anxiety, pain, inflammation, addiction, an autoimmune disease, an eating disorder, or an obsessive compulsive disorder.

44. The method of any one of claims 17-42, wherein the disease is depression, anxiety, pain, inflammation, or addiction.

45. The method of any one of claims 17-42, wherein the disease is anxiety.

46. The method of any one of claims 17-42, wherein the disease is pain.

47. The method of any one of claims 17-42, wherein the pain is treatment resistant pain.

48. The method of any one of claims 17-42, wherein the the pain is opioid resistant pain.

49. The method of any one of claims 17-42, wherein the the pain is opioid resistant refractory pain.
